SEQUENCE LISTING (83 sequences)

SEQ ID NO: 1 - DNA, 1155 bp, Brassica napus
atgggtgcaggtggaagaatgcaagtgtctcctccctccaagaagtctgaaaccgacaccatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatga

SEQ ID NO: 2 - DNA, 1155 bp, Brassica napus
atgggtgcaggtggaagaatgcaagtgtctcctccctccaaaaagtctgaaaccgacaacatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccagggctgcgtcctaaccggcgtctgggtcatagcccacgagtgcggccaccacgccttcagcgactaccagtggctggacgacaccgtcggcctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggcctttgtacttagccttcaacgtctcggggagaccttacgacggcggcttcgcttgccatttccaccccaacgctcccatctacaacgaccgtgagcgtctccagatatacatctccgacgctggcatcctcgccgtctgctacggtctctaccgctacgctgctgtccaaggagttgcctcgatggtctgcttctacggagttcctcttctgattgtcaacgggttcttagttttgatcacttacttgcagcacacgcatccttccctgcctcactatgactcgtctgagtgggattggttgaggggagctttggccaccgttgacagagactacggaatcttgaacaaggtcttccacaatatcacggacacgcacgtggcgcatcacctgttctcgaccatgccgcattatcatgcgatggaagctacgaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatga

SEQ ID NO: 3 - DNA, 1155 bp, Brassica napus
atgggcgcaggtggaagaatgcaagtctctcctccctccagctcccccgaaaccaaaaccctcaaacgcgtcccctgcgagacaccacccttcactctcggagacctcaagaaagcaatcccacctcactgcttcaaacgctccatccctcgctccttctcctacctcctcttcgacatcctcgtctcctcctccctctaccacctctccacagcctacttccctctcctcccccaccctctcccttacctcgcctggcccctctactgggcctgccaaggctgcgtcctaacgggcctctgggtcatcgcccacgaatgcggccaccacgccttcagcgaccaccagtggctggacgacgccgtgggcctcgtcttccactccttcctcctcgtcccttacttctcctggaagtacagccatcgacgccaccattccaacaccggatccctcgagagggatgaagtgttcgtccccaagaagaaatccgacatcaagtggtacggaaagtacctcaacaacccgctaggacgcacggtgatgctaaccgtccagttcacgctcggctggccgttgtacttagccttcaacgtctctggaagaccttacagcgacggtttcgcttgccatttccacccgaacgctcccatctacaacgaccgcgagcgtctccagatatacatctctgacgctggcgtcctctccgtatgttacggtctctaccgctacgctggttcgcgaggagtggcctcgatggtctgtgtctacggagttccgcttatgattgtcaactgtttcctcgtcttgatcacttacttgcagcacacgcacccttcgctgcctcactatgattcttcggagtgggattggttgagaggagctttggctactgtggatagagactatggaatcttgaacaaggtgtttcataacatcacggacacgcacgtggcgcatcatctgttctcgacgatgccgcattataacgcgatggaagcgaccaaggcgataaagccgatacttggagagtattaccagtttgatggaacgccggtggttaaggcgatgtggagggaggcgaaggagtgtatctatgttgaaccggataggcaaggtgagaagaaaggtgtgttctggtacaacaataagttatga

SEQ ID NO: 4 - DNA, 1141 bp, Brassica napus
atgggcgcaggtggaagaatgcaagtctctcctccctccagctcccccggaaccaacaccctcaaacgcgtcccctgcgagacaccaccattcactctcggagacctcaagaaagcaatcccacctcactgcttcaaacgctccatcccacgctccttctcctcttcgacatcatcatctcctcctcggctcctccctctaccacctctccacagcctacttccctctcccttacctcgcctgacccctctactgggcctgccaaggctgcgtcctaacgggcctctgggtcatagcccacgagtgcggccaccacgccttcagcgaccaccagtggctggacgacgccgtcggcctcgtcttccactccttcctcctcgtcccgtacttctcctggaagtacatccatgacgccaccattccaacaccggatccctcgatagggacgaagtgttcgtccccaagaagaaatccgacatcaagtggtacggcaagtacctcaacaacccgctaggacgcacggtgatgctaaccgtccagttcaagctcggctggccgttgtacttagccttcaacgtctcgggaagaccttacagcgacggtttcgcttgccatttccacccgaacgctcccatctacaacgaccgcgagcgtctccagatatacatctctgacgctggcgtcctctccgtatgttacggtctctaccgttacgctgcttcgcgaggagtagcctctgtggtctgtgtctacggagttccgcttctaattgtcaactgtttcctcgtcttgatcacttacttgcagcacacgcacccttcgctgcctcactatgattcttccgagtgggattggttgagaggagctttggctactgtggatagagactatggaatcttgaacaaggtgttccataacatcacggacacgcacgtggcgcatcatctgttctcgacgatgccgcattataacgcgatggaagcgaccaaggcgataaagccgatactttggagagtattaccagtttgatggaacgccggcggttaaggcgatgtggagggaggcgaaggagtgtatctatgttgaaccggataggcaaggtgagaagaaaggtgtgttctggtacaacaataagttatga

SEQ ID NO: 5 - protein, 384 aa, Brassica napus
MGAGGRMQVSPPSKKSETDTIKRVPCETPPFTVGELKKAIPPHCFKRSIPRSFSYLIWDIIIASCFYYVATTYFPLLPHPLSYFAWPLYWACQGCVLTGVWVIAHECGHHAFSDYQWLDDTVGLIFHSFLLVPYFSWKYSHRRHHSNTGSLERDEVFVPKKKSDIKWYGKYLNNPLGRTVMLTVQFTLGWPLYLAFNVSGRPYDGGFACHFHPNAPIYNDRERLQIYISDAGILAVCYGLFRYAAAQGVASMVCFYGVPLLIVNGFLVLITYLQHTHPSLPHYDSSEWDWLRGALATVDRDYGILNKVFHNITDTHVAHHLFSTMPHYHAMEATKAIKPILGEYYQFDGTPVVKAMWREAKECIYVEPDRQGEKKGVFWYNNKL

SEQ ID NO: 6 - protein, 384 aa, Brassica napus
MGAGGRMQVSPPSKKSETDNIKRVPCETPPFTVGELKKAIPPHCFKRSIPRSFSYLIWDIIIASCFYYVATTYFPLLPHPLSYFAWPLYWACQGCVLTGVWVIAHECGHHAFSDYQWLDDTVGLIFHSFLLVPYFSWKYSHRRHHSNTGSLERDEVFVPKKKSDIKWYGKYLNNPLGRTVMLTVQFTLGWPLYLAFNVSGRPYDGGFACHFHPNAPIYNDRERLQIYISDAGILAVCYGLYRYAAVQGVASMVCFYGVPLLIVNGFLVLITYLQHTHPSLPHYDSSEWDWLRGALATVDRDYGILNKVFHNITDTHVAHHLFSTMPHYHAMEATKAIKPILGEYYQFDGTPVVKAMWREAKECIYVEPDRQGEKKGVFWYNNKL

SEQ ID NO: 7 - protein, 384 aa, Brassica napus
MGAGGRMQVSPPSSSPETKTLKRVPCETPPFTLGDLKKAIPPHCFKRSIPRSFSYLLFDILVSSSLYHLSTAYFPLLPHPLPYLAWPLYWACQGCVLTGLWVIAHECGHHAFSDHQWLDDAVGLVFHSFLLVPYFSWKYSHRRHHSNTGSLERDEVFVPKKKSDIKWYGKYLNNPLGRTVMLTVQFTLGWPLYLAFNVSGRPYSDGFACHFHPNAPIYNDRERLQIYISDAGVLSVCYGLYRYAGSRGVASMVCVYGVPLMIVNCFLVLITYLQHTHPSLPHYDSSEWDWLRGALATVDRDYGILNKVFHNITDTHVAHHLFSTMPHYNAMEATKAIKPILGEYYQFDGTPVVKAMWREAKECIYVEPDRQGEKKGVFWYNNKL

SEQ ID NO: 8 - protein, 373 aa, Brassica napus
MGAGGRMQVSPPSSSPGTNTLKRVPCETPPFTLGDLKKAIPPHCFKRSIPRSFSSSTSSSPPRLLPLPPLHSLLPSPLPRLTPLLGLPRLRPNGPLGHSPRVRPPRLQRPPVAGRRRRPRLPLLPPRPVLLLEVHPRHHSNTGSLDRDEVFVPKKKSDIKWYGKYLNNPLGRTVMLTVQFKLGWPLYLAFNVSGRPYSDGFACHFHPNAPIYNDRERLQIYISDAGVLSVCYGLYRYAASRGVASVVCVYGVPLLIVNCFLVLITYLQHTHPSLPHYDSSEWDWLRGALATVDRDYGILNKVFHNITDTHVAHHLFSTMPHYNAMEATKAIKPILWRVLPVWNAGGGDVEGGEGVYLCTGAREERCVLVQQVM

SEQ ID NO: 9 - DNA, 1155 bp, Brassica napus; identical to SEQ ID NO: 1

SEQ ID NO: 10 - DNA, 1154 bp, Brassica napus
atgggtgcaggtggaagaatgcaagtgtctcctccctccaaaaagtctgaaaccgacaacatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccattacttccctctcctccctcaccctctctcctacttcgcctggcctctctactgggcctgccagggctgcgtcctaaccggcgtctgggtcatagcccacgagtgcggccaccacgccttcagcgactaccagtggctggacgacaccgtcggcctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggcctttgtacttagccttcaacgtctcggggagaccttacgacggcggcttcgcttgccatttccaccccaacgctcccatctacaacgaccgtgagcgtctccagatatacatctccgacgctggcatcctcgccgtctgctacggtctctaccgctacgctgctgtccaaggagttgcctcgatggtctgcttctacggagttcctcttctgattgtcaacgggttcttagttttgatcacttacttgcagcacacgcatccttccctgcctcactatgactcgtctgagtgggattggttgaggggagctttggccaccgttgacagagactacggaatcttgaacaaggtcttccacaatatcacggacacgcacgtggcgcatcacctgttctcgaccatgccgcattatcatgcgatggaagctacgaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatga

SEQ ID NO: 11 - DNA, 1141 bp, Brassica napus
  misc_feature (697)..(722): n is a, c, g, or t
atgggcgcaggtggaagaatgcaagtctctcctccctccagctcccccggaaccaacaccctcaaacgcgtcccctgcgagacaccaccattcactctcggagacctcaagaaagcaatcccacctcactgcttcaaacgctccatcccacgctccttctcctcttcgacatcatcatctcctcctcggctcctccctctaccacctctccacagcctacttccctctcccttacctcgcctgacccctctactgggcctgccaaggctgcgtcctaacgggcctctgggtcatagcccacgagtgcggccaccacgccttcagcgaccaccagtggctggacgacgccgtcggcctcgtcttccactccttcctcctcgtcccgtacttctcctggaagtacatccatgacgccaccattccaacaccggatccctcgatagggacgaagtgttcgtccccaagaagaaatccgacatcaagtggtacggcaagtacctcaacaacccgctaggacgcacggtgatgctaaccgtccagttcaagctcggctggccgttgtacttagccttcaacgtctcgggaagaccttacagcgacggtttcgcttgccatttccacccgaacgctcccatctacaacgaccgcgagcgtctccagatatacatctctgacgctggcgtcctctccgtatgtnnnnnnnnnnnnnnnnnnnnnnnnnngcgaggagtagcctctgtggtctgtgtctacggagttccgcttctaattgtcaactgtttcctcgtcttgatcacttacttgcagcacacgcannctncnctgcctcactatgattcttccgagtgggattggttgagaggagctttggctactgtggatagagactatggaatcttgaacaaggtgttccataacatcacggacacgcacgtggcgcatcatctgttctccacgatgccgcattataacgcgatggaagcgaccaaggcgataaagccgatactttggagagtattaccagtttgatggaacgccggcggttaaggcgatgtggagggaggcgaaggagtgtatctatgttgaaccggataggcaaggtgagaagaaaggtgtgttctggtacaacaataagttatga

SEQ ID NO: 12 - protein, 384 aa, Brassica napus; identical to SEQ ID NO: 5

SEQ ID NO: 13 - protein, 373 aa, Brassica napus
MGAGGRMQVSPPSKKSETDNIKRVPCETPPFTVGELKKAIPPHCFKRSIPRSFSYLIWDIIIASCFYYVATITSLSSLTLSPTSPGLSTGPARAASPASGSPTSAATTPSATTSGWTTPSASSSTPSSSSLTSPGSTVIDATIPTLAPSRETKCLSPRRSQTSSGTASTSTTLWDAPCRFSSLSAGLCTPSTSRGDLTTAASLAISTPTLPSTTTVSVSRYTSPTLASSPSATVSTATLLSKELPRWSASTEFLFLSTGSFSLTCSTRILPCLTMTRLSGIGGELWPPLTETTESTRSSTISRTRTWRITCSRPCRIIMRWKLRRRSRYWESIISSMGRRWLRRCGGRRRSVSMWNRTGKVRRKVCSGTTISY

SEQ ID NO: 14 - protein, 373 aa, Brassica napus
  misc_feature (232)..(240): Xaa can be any naturally occurring amino acid
MGAGGRMQVSPPSSSPGTNTLKRVPCETPPFTLGDLKKAIPPHCFKRSIPRSFSSSTSSSPPRLLPLPPLHSLLPSPLPRLTPLLGLPRLRPNGPLGHSPRVRPPRLQRPPVAGRRRRPRLPLLPPRPVLLLEVHPRHHSNTGSLDRDEVFVPKKKSDIKWYGKYLNNPLGRTVMLTVQFKLGWPLYLAFNVSGRPYSDGFACHFHPNAPIYNDRERLQIYISDAGVLSVCXXXXXXXXXRGVASVVCVYGVPLLIVNCFLVLITYLQHTXXXLPHYDSSEWDWLRGALATVDRDYGILNKVFHNITDTHVAHHLFSTMPHYNAMEATKAIKPILWRVLPVWNAGGGDVEGGEGVYLCTGAREERCVLVQQVM

SEQ ID NO: 15 - DNA, 1166 bp, Brassica napus
ttacgtcctaatcatcgtgtctctggtcgtcttaattgtgcggagaacgcaaaaagcaatcccaccgcatgctcccctcgatccctcgctctttctcctacctcatctggcgacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttaacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgagggagctttggctaccgttgacagagactacggaatcttgaacaagtcttccccaatattaccgacccgcacgtggcgcatcatctgttctccacgatgccgcattatccacgcgatggaaagctaccaagccgataaagccgaatactgggagaagttttatcagttcgatggacgccggtggttaggcgaatgtgaggaaggcgaagaatgtatctattgttgaaccgaacaggccagtggaagaaggtgtgtgttctgttcaacaataagttatgaggattgatgatggtgaaggacaagcaagatattgtcacgacctttttcttgcttgtctctgtgggtcgcttctgga

SEQ ID NO: 16 - DNA, 1164 bp, Brassica napus
ttgacatctgggtctgagtgatctgcgagaaccggccttcacggtcggagaagtcaagaaagcaatcccaccgcactgttcaacctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcgacttcgcttgccatttccaccccaacgctcccatctacaacgaccgcgagcgtctccagatatacatctccgacgctggcatcctcgccgtctgctacggtctcttccgttacgccgccgcgcaggagtggcctcgatggtctgcttctacggagtcccgcttctgattgtcaatggtttcctcgtgttgatcacttacttgcagcacacgcatccttccctgcctcactacgattcgtccgagtgggattggttgaaggggggctttggctaccgtgacgaagactacggaattttgaacaaggtcttccacaatattaccgaacaggcacggtggcggcatcatctgttcctccacgatgcccgcattatccacggcgaatggagctctaccagggcgataagtcgaaatcttggggagagtatattatccagttcgatgggacgccggtggttaaggattgtgaaggaggcagggatgtgtattctaggggaccgacgcaggttgaagaagggtggttctgtcaaacatagtagagaggatagtatgtgtagacaggatatttgtacgaactttctttgggtcttggctgcgtcttgaaga

SEQ ID NO: 17 - DNA, 1179 bp, Brassica napus
cctaaacgtctcgtttgtgacgatgcgcgagcgggccttcaggtcggaagctcaagaaggcgatcccaccgcatgttcaacactcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgagaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtatatcagttcgatgggacgccggtggttaaggcgatgtggagggaggcgaaggagtgtatctatgtggaaccggacaggcaaggtgagaagaaaggtgtgttctggtacaacaaatagttatgagatatgatgatgtgaaggaacaaggaagaaatttgtcacgaaccctttcctcttgctgtcctcctggggtgcgctcctctgaa

SEQ ID NO: 18 - DNA, 1170 bp, Brassica napus
gacggttcatttaagcgcgtattgtgcgagacaccgcccttcactgtcggagaactcaagaaagcaatcccaccgcattgttttaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacgatctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtggattggttgaggggagctttggctaccgttgacagagactacggaatcttgaacaaaggtcttccacaatattaccggacacgcacgtggcgcatcatctgttctccacgatgccgcattatcacgcgatggaagctaccaaggcgataaagccgatactggggagagtattatcagttcgatgggaacgcggtacaggcgatgtgaaggaggcgaaaggatgtatcctatgtggaaaccggacgccaagtgagaagaatggtgtgttcggtacaacaaatagttatgagtattggatgatggtgcagagatcagagattggtccgaacattcctgcctgtctcggtcgcctctggacc

SEQ ID NO: 19 - DNA, 1205 bp, Brassica napus
cccctcccgtcaggtcgcgtgccctgcgagaaccgcccttcactgtcggagaactcaagaaagcaatcccagcgggtgttgcaaggcgcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcaacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatctttgaacaaggtcttccacaatattaccgacacgcacgtggcgcatcattctgttctccacgatgccgcattatcacgcgatggaagctaccaaaggcgataaaacccggatactgggagaagtattattcagttccgatgggacgctggtggtttaagggcgatggtggagggagtgcgaaaggagtgaattcttatgtttgaacccggacacagggcaaaggtggaagaaagaaaggtggtgttcttggttacaacataagttatggaggaatattgaatgaatggtgaaagacagagatttgtacggacgttctctctgctgtctctggtgccgctccttgtga

SEQ ID NO: 20 - DNA, 1148 bp, Brassica napus
ccaacttcagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgtcgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaaggtcttccacaatattaccgacacgcacgtggcgcatcatctgttctccacgatgccgcattatcacgcgatggaagctaccaaggcgataaagccgatactggagagtattatcagttcgatgggacgccggtggttaagcgatgtgaggagcgtgagtgtatcttatgtggaccggacagcagtgagaagaaagtgtgtctgttacaccaatagtatgaagatatgatgatgtgagacagagatttgtcacgaaccttcctgcgccgggtggcccctgtga

SEQ ID NO: 21 - DNA, 1133 bp, Brassica napus
ccgaacatcagcgcgtacctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctttctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaaggcgataaagccgatactgggagagtatttatcagttcgatgggacgccggtggttaagcgatgtgagggagcgaagagtgtatctatgtgaccggacagcagtgagaagaagtgtgtctgtacacattagtatgaagatatgatgatgtgagacagagatttgtcacgaactttctctgcttc

SEQ ID NO: 22 - DNA, 1186 bp, Brassica napus
gaccaaaattcagcgcgtactctgcgagaaccgcccttcactgtcggagaagtcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgctttcagcgactaccagtagcttgacgacaccgtcggtctcatcttccctctttcctcctcgtcccttacttctcctggaagtacagtcatcgacgccaccattccaacactggctccctcgagagagacgaagtgtttgtcccccagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattgggttgaggggagctttgggctatcgttgacagagactacggaatcttgaacaaggtgtttccacaatattaccgacacgcacgtggcgcatcatctgttctccacgatgccgcattatcacgcgatgggaagctactcaagcgataaagccgaatactggggagagtatttattcagttcgatggacgccgtggttagcgatggtggagggaggcgaggagtgtatcttattgttggaatgggacaggtcaagggagaagacgggtgtgtttctggtacaacaattagctatgaggaatggatggattgggtgaggaactatgagattttggttatcgaagctttctctgagctgtccatcggtgccgacta

SEQ ID NO: 23 - DNA, 1132 bp, Brassica napus
aggagcgcgccgtcatcgcgtatctgcgagaaccgcccttcacggtcgggaagtcaagaaagcaatcccaccgcactgttgcaaggcgcgatcgctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcataacccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcaaggagtggcctcgatggtctgcttctactgagtcccgcttctgattgtcaatggtttcctcgtgttgatcacttacttgcagcacacgcatccttccctgcctcactacgaattcgtccgaatgggattggttgaggggagctttggctaccgttgacagagactacggaatcttgaacaggtctttcccaatattaccgacacgcacgtggcgcatcatctgttctcacgatgcgcattattacgcgatggaagctaccaaggcgattaagcgaataactgggggagtttattcagtccattgacgccgtcgtttatgcgatgtgagaggcagatgtattctattgttgacggacgccaagtggagagttgtctggtcacatagttataagaatgatgatgtagacagagattgtacgacctccatgcgactagtcgtccttgaa

SEQ ID NO: 24 - DNA, 1186 bp, Brassica napus
aggtggtcagccgtcgttgcgagccctgcgagaacggcccttgcggtcgcggagtgaagggaggagcccagcgggtggtgcaaggcggggtcggtcgctctttctccggcgggaggtgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttccttctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcctgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatgggaagctaccaaggcgaaaaagccgatactggggagaagtattatcagttcgatgggacgccgtggtaagccgatgtggagggaggcgatgggagtggtatctatgttgaaccggacaggcaggtgagaaagaaaagggtggtggttcttggttacaacaattaggtaatggaggatatgatgatgcttaagaacaagaaatttgttcacgaaccctttctccttgctgtcctgggcggccttgtaaa

SEQ ID NO: 25 - DNA, 1151 bp, Brassica napus
ggggacgtcagtcgcgtaccgtgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccgacgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagaaagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatgggacgccggtggtttaaggcgatgtggagggaggcgaaggagtgtatctatgtggaaccggacaggcaagtgagaagaaaggtgtgtctggtacacatagtatgagatatgatgatgtgaagacaagaagatttttgtcccgaaccttctctcttgctcc

SEQ ID NO: 26 - DNA, 1175 bp, Brassica napus
gccgaggagtgagtcgcgtacgctgcgagacaccgcccttcactgtcggagaagtgaagaaagcaatcccagcgcactgggcaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttttcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatgggacgccggtggttaaggcgatgtggagggaggcgaagagtgtatctatgtggaaccggaacaggcaaggtgagaagaaaggtgtgtctggtaacaacaataagtatgagatatgatgatgtgaagaacaaagaagatttgtcacgaactttctcttgctgtcctccgggggccgcctctgtaa

SEQ ID NO: 27 - DNA, 1165 bp, Brassica napus
tcctgccacgtcgtcgcgtaccctgcgagaaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccactccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaaggtcttccacaatattaccgacacgcacgtggcgcatcatctgttctccacgatgccgcattatcacgcgatggaagctaccaaggcgataaagccgatactggagagtattatcagttcgatgggacgccggtggttaaggcgatgtgaggagcgaaggagtgtatcttatgtggaacggacaggcaaggtgagaagaaaggtggtgtctgtacaaccaatagtatgagatatgatgatgtgaggacaggagattggtcacggacctttctctgcttctcgtgggcctcctgtgaa

SEQ ID NO: 28 - DNA, 1175 bp, Brassica napus
tggagcgaaagtcagtcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcagcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatgggacgccggggttaaggcgatgtgagggaggcgaaggagtgtatctatgtggaaccggacaggcaagtggagaagaaaagtgtgttctggtacaacaataagtatgagatatgatgatggtgaaaggacaagagatatgtcacgaacgttcttctgctgtctctgggcgcgcctcttgga

SEQ ID NO: 29 - DNA, 1176 bp, Brassica napus
gcgaacgtcagtcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacactcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaaggtcttccacaatattaccgacacgcacgtggcgcatcatctgttctccacgatgccgcattatcacgcgatggaagctaccaaggcgataaagccgatactgggagagtattatcagttcgatgggacgccggtggttaaggcgatgtgagggaggcgaaggagtgtatcttatgtggaaccggacaggcaaggtgagaagaaggtgtgtctggtacaacaataagtatgagatatggatgatgtgaagaacaaagaagaaattgtcacgaactttctcttgctgtcctcggtgtgcccgctctgtga

SEQ ID NO: 30 - DNA, 1154 bp, Brassica napus
ggctcttcatcagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctactacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtgccgcatcatctgttctccacgatgccgcattatcacgcgatggaagctaccaaggcgataagccgatactggggagagtatttatcagttcgatgggacgccggtgataggcgatgtgagggaggcgagatgtattctatgtggaccgacaggcaagtggaagaggtgtgtctgtacacatagtatgagaatgatgatgtgaagaacagaagatttgtcacgacttcccttgcgtctcgcggcgccctcgtgagaa

SEQ ID NO: 31 - DNA, 1165 bp, Brassica napus
gacgcaccgtcagtcgcgtacctgcgagacaccgcccttcactgtcggagaagtcaagaaagcaatcccaccgcactgtttcaaaggctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcctgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatggacgccggtggtttaaggcgatgtggagggaggcgaagagtgtatctatgtggaaccggacaggcaaggtgagaagaaaggtgtgttctggtacaacaatagtatgagaatgatgatgtgagacagagatttgtcacgaaccttctcttgctgctctcgggcgcgcctgaaaa

SEQ ID NO: 32 - DNA, 1163 bp, Brassica napus
tacgacgatcagtcgcgtaccctgcgagaaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaaagctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatgggacgccggtggttaagcgatgtggagggaggcgaagagtgtatctatgtggaaccggacaggcaaggtgagaagaacgtgtgtctgtacacataggtatgagatatgatgatgtgaagacaagagattgtcacgacttctctgctgtctctggtgcgccccctctgaggaa

SEQ ID NO: 33 - DNA, 1173 bp, Brassica napus
cacgacggtcgtcgcgtaccctgcgagaaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctagcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatgggaagctaccaaaggcgataaagccgatactggagagtattatcagttcgatgggacgccggtggttaaaggcgatgtgaagggaggcgaaggagtgtatcttatgtggaaacggacaggccaagtgaaaagaaaggtgtgtctggtacaaacaataaagttatgagatatgatgatgtgaaagaaccaagagaattgtcacgaacctttcctgctgtcctggtgcgcccttgaa

SEQ ID NO: 34 - DNA, 1159 bp, Brassica napus
gggagcgtcagtcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccatttcaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatataccgacacgcacgtggcgcatcatctgttctcacgatgccgcattatcacgcgatggaagctaccaaggcgataaagcgatactggggaggagtattattcagttcgatgggacgccggtggttaagcgatgtggagggaggcgaagatgtatctatgtgacggacaggccagtggagaagaaggtgtgttctgttacaaccataggtatgagattgatgatgtgaagacaagaagaatttggtaccgaccttcctgctgtctggctggggtcttgga

SEQ ID NO: 35 - DNA, 1165 bp, Brassica napus
ccccacgtcagtcgcgtaccctgcgagaaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctatcgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatgggacgccggtggttaagcgatgtggagggaggcgaagagtgtatctatgtggaaccggacaggcaagtgagaagaaagtgtgtctggtacaacaattagtatgagatatgatgatgtgagaccagagatctgtcacgaactttctctgctgtctctcgtgcgcgctctcgtggaa

SEQ ID NO: 36 - DNA, 1183 bp, Brassica napus
gcagacggtgcagtcgcgtattgtgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccactacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaaaagggtcttccacatattaccgacacgcacgtggcgcatcatctgttctccacgatgccgcattatcacgcgatggaagctaccaggcgataaagccgatactggggagagtatttatcagttcgatgggacgccggtggttaggcgatgtggagggaggcgaggagtgtattctatggtggacccggacagcagggtgagaagaaaggttgtgtgttctggtacacaataaggttatgaagatatggatgatggtgagaccaggagatttgttcaagactttctcccttgccgtctcggtgcgcctctctgttggaa

SEQ ID NO: 37 - DNA, 1215 bp, Brassica napus
cgacacggcagtcgagagctctgggagaaccgcccttcacggtcggagagtgaagaaaggatcccaccggggtggcgcaagggggggtcgctcgctctttctcctgcgggaggtggaacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcaggaagtggcctcgatggtctgcttctacggagtcccgcttctgatttgtcaatggtttcctcgtgttgatcacttactttgcagcacacgcatccttccctgccctcactacgattcgtccgagtgggattggttgaggggagctttggctaccgtttgacagagactacggaatctttgaacaaggtctttccacaatattaccgacacgcacgtggcgcatcatctgttctccacgatgccgcattatcacgcgatggaagctaccaaggcgataaagccgatactggcagagtattatcaatttcgatgggacgccggtgggttacggcgatgttgcatggaggccgaggagtgtaatcttattgtggaatccggtacagggcaagtggagaagaaaggtgttgttctggtacaccaataagttattgaggatattgatgatatgggtgtaggaaccaaacgaagaaattttgttcaccgataccttttctctcttggcctgtctccctcggtctcgtcctctggaacta

SEQ ID NO: 38 - DNA, 1179 bp, Brassica napus
tgcccgcgtcagcgcgtaccctgcgagaaccgcccttcactgtcggagaactcaagaaagcaatcccaccgcactgtttcaaacgctcgatccctcgctctttctcctacctcatctgggacatcatcatagcctcctgcttctactacgtcgccaccacttacttccttctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactgggagagtattatcagttcgatgggacgccggtggttaaaggcgatgtgtagggaggcgaagagtgtatctatgtgggaaccggacaggcaggtgagaagaaaggtggtgtctggtacacatagttatgaggatatggatgatggtgaaagacaagagattctgtcaacgatcatttctgtcttgactgtcctggggcccctcttgtggaaa

SEQ ID NO: 39 - DNA, 1179 bp, Brassica napus
ccgcacgtcagtcgcgtaccgtgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgagcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaaagctaccaaggcgataaagccgatactgggagagtattatcagttcgatgggacgcccggtggtttaaggcgatgtgagggaggcgaaggagtgtatctatgtggcaccggacaggcaaggtggagaagaaaggtgtgtctggtacaacaataagttatgaagatatgatgatgttgaaagacaaggaggatatgtcacgatcttctctgcgttctcggggggccctcttctgtgaa

SEQ ID NO: 40 - DNA, 1215 bp, Brassica napus
tattcatcctcggctctgatgtgcttgagagaacgggccttgcggtcgggaagtcaagaaagcaatcccaccggggtggtcaacactcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacaagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgatttcgtccgagtgggattggttgaggggagctttggctaccgttgacagagactacggaatctttgaacaaggtctttccacaatattaccgacacgcacgtggcgcatcatctgttctccacgatgccgcattatcacgcgatgggaagctaccaagggcgataaagccgatactgggagagtattatcagtttcgatgggacgccgggtggttaaaggcgatgtggagggaggcggaaggagttgttattcttattgttggaactcgggacaggccaaggtggagaaagaaagggtgtgtttctggttacaacaattaaagttaatggagggatatgaatgattgctgaaagaacaaggagatatttgttcacgaacctttctctctttgctgtctctttgggtgcgctctcttgaa

SEQ ID NO: 41 - DNA, 1136 bp, Brassica napus
gagttacattcagtcgcgtattgagcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcagtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacatattaccgacacgcacgtggcgcatcatctgtctccacgatgccgcattatcacgcgatggaagctaccaggcgataaagccgatactggggaggagtattatcagtcgatggacgccgtgtagcgatgtggagggaggcgacggatgtatctatgtgaccgacagcaggtgagaagaaggtgtgtctgtacacattaggttatgaagaatgatgatggtgaggacaggaggattttgtcacgactttccttgcgtc

SEQ ID NO: 42 - DNA, 1102 bp, Brassica napus
ggcggacaatcagtcgcgtaccctgcgagacaccgcccttcactgtcggagaactcaagaaagcaatccagcggctgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatattaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcaggagtggcctcgatggtctgctctacggagtcccgcttctgattgtcaatggtttcctcgtgtgatcactactgcagcaacacgcatctccctgcctcactacgatcgtccgagtggatggtggagggggagcttggctaccgttgacaggagaactacggattcttgaacaaggtcttccaccaataattaccgaacaccgccaacgtgggcccatcacatcgttctccccacgaatggcccgcattaatcacgcgattggagccttccccaggggataaagcgcgatatctctggggaggattatatccagtccaattgggaatccggtttagccaatgtgaaggaaggccaagagttttcctattgggaacggaacggctgtgagaaaaggtgttcgtcacacataagctttg

SEQ ID NO: 43 - DNA, 1148 bp, Brassica napus
gacggcagtcagtcgcgtaccctgcgagacaccgcccttcactgtcggagaactcaagaaagcaatccagcgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagttatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcaggagtggccttcgatggtctgcttctacggagtcccgcttctgattgtcaatggtttcctcgtgttgatcacttacttgcagcacacgcatccttccctgcctcactacgattcgttcgaggtggggattggttgagggggagctttggcctaccgttgaacagagagacttacgggaattcttgaacaaggttcttccaacaattatttaccgacacgcacggtgggccgcattcatcctgtttctctccacgatggcccgcaattattcacgcgcgttggaaagcctaacaacggcgataaaaggccggataccttggggagagatatataatcagtttccaaatgggaacgccggtgtgtttaggcaattgttgaggggggcaaagatgttattctaatgtgtgaaccgacatggcaaggtgtggaaaaacgtgtgttccgtcaacaataagtttagac

SEQ ID NO: 44 - DNA, 1183 bp, Brassica napus
ccgaacatcagcgcgtaccctgcgagacaccgcccttcactgtcggagaactcaagaaagcaatcccacgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtacgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatctgacaaggtcttccacaatattaccgacacgccacgtggcgcatcatctgttcctcacgatgccggcattatccacgcgatggaagcttaccaaggcgaataaagccggataactgggatgagtaattatcagttcgaatgggaaccgcccgttgttaaggcattgtgcagggtaggcgaaggaatgtatctatgtgtaacctggacaggctagtggagaaaaaggggtgttctggtacacaaaagatagaggcttcgagatggctcacaacacctacaatctgtgactcgatcctctctatgcagtaccctggctgtcctctggat

SEQ ID NO: 45 - DNA, 1166 bp, Brassica napus
attcgggcgttcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctgaccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaggcgataaagccgatactggggaggagttattatcagttcgatgggacgccggtggtagcgatgtggagggaggcgatatgtattctattgtgaccggacagcaggtgagagaaggtggtgttctgtacacattagtatgaagattgatgatgtgaagacaagaaggatttgtcacgacttccttgcgtcctggtggtggtgcctctgtggaa

SEQ ID NO: 46 - DNA, 1152 bp, Brassica napus
ggcgaacatcagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgtttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgagggagctttggctaccgttgacagagactacggaatcttgaacaaggtcttccacatattaccgacacgcacggtggcgcatcatcttgttcttcccgatgccgcattatcacgcgatggaagctaccaaggcgataaagccgatactgggaagagttatttattcagttcgatgggacgccggtggttaaggcgattgtggagggaggcgaggagtgttattcttatggtggaccgacagcaggtgagaagaagggtgttgtttctgtacacaatagttatgaagaatgatgatgggtcagacagagatttgttcacggactttccttgcg

SEQ ID NO: 47 - DNA, 1149 bp, Brassica napus
gagaaaaatcagcgcgtaccctgcgagacaccgcccttcactgtcggagaactcaagaaagcaatcccaccgcatgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgagggagctttggctaccgttgacagagactacggaatcttgaacaagtcttccacaatattaccgacacgcacgtggcgcatcatctgttctccacgatgccgcattatccacgcgatggaagctaccaaggcgaataaagccgatactgggagagtattatcagttcgatgggacgccggtggttaaggcgatgtggaggagcgacgatgtatctattgtgaccgaatcaggtgagaagaaggtgtgttctgttacacatagtatgaagaattgatgatggtgaggaccagagtttgttccgaacttccctgcgtccggggctccttgga

SEQ ID NO: 48 - DNA, 1170 bp, Brassica napus
cccgttcattcagcgcgtaccctgcgagacaccgcccttcactgtcggagaactcaagaaagcaatcccaccgcatgtttcaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacaccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgagggagctttggctaccgttgacagagactacggaatcttgaacaagtcttccacaatattaccgacacgcacgtggcgcatcatctgttctcacgatgccgcattatcacgcgatggaaagctaccaaggcgataaagccgatactggggagagtattatcagttcgatgggacgccggtggttaggcgatggtggagggaggcgaaggatgtattctatggtggacggacaggcaggtgagaagaaggtgtgtgttctgttacaaacatagtatgagaaatgatgattgtgagaccaagagatttgtcaccgaaccttttccttgcgtcatcgggtgggtccttctgaga

SEQ ID NO: 49 - DNA, 1168 bp, Brassica napus
gcgtacagtcagcgcgtaccctgcgagacaccgcccttcactgtcggagaactcaagaaagcaatcccaccgcaatgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcagccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatcttgttctccacgatgccgcattatcacgcgatggaagctaccaaggcgataaagccgatactggggaggagtattatcagttcgatgggacgccggtggttaaggcgatgtgagggaggcgagatgtatctatgtggaccggacaggcaggtgagaagaaggtgttgttctgtacaacatagttatgaagatatgatgatgggtgaagaacagaagatttgtcacgatcgttccttgcgcttgggtggtgccctggtgaga

SEQ ID NO: 50 - DNA, 1149 bp, Brassica napus
cccaaactggcagtcgcgtaccctgcgagacaccgcctttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcgactggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctcacgatgccgcattatcacgcgatggaagctaccaaggcgataaagccgatactgggagagtattatcagttcgatgggacgcggtggctatgcgatgtgagggaggcgacgatgtatctatgtgaacggacagccagtgagaagaacgtgtgttctgttacaacatagtatgaagaaatgatgatgtgaagacagagattgtcacgacttcctgcgtcttgggtgctcctgga

SEQ ID NO: 51 - DNA, 1178 bp, Brassica napus
gcccgcgttcagtcgcgtacctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaagggtgcgtcctaaccggcgtctgggtcatagcccacgagtgcggccaccacgccttcagcgactaccagtggcttgacgacaccgtcagtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaaggtcttccacaatattaccgacacgcacgtggcgcatcatctgttctccacgatgccgcattatcacgcgatggaagctaccaaggcgataaagccgatactgggagagtattatcagttcgatgggacgccggtggttaagcgatgtggagggaggcgaaggagtgtatctatgtggaaaccggaacagcagtgagaagaaccgtggtgttctgtaacaacaatagtatgaggatatgatgatgggtgaaagacaagaagatatgtcacgatccctttctctgctgtcgtgcgtgggtggctctctgtga

SEQ ID NO: 52 - DNA, 20 bp, Artificial Sequence
  Nucleic acid sequence fragment
gtctcctccctccaaaaagt

SEQ ID NO: 53 - DNA, 18 bp, Artificial Sequence
  Nucleic acid sequence fragment
gtgtctcctccctccaaa

SEQ ID NO: 54 - DNA, 20 bp, Artificial Sequence
  Nucleic acid sequence fragment
ctacagaaacaaacatgggc

SEQ ID NO: 55 - DNA, 18 bp, Artificial Sequence
  Nucleic acid sequence fragment
gtctctcctccctccagc

SEQ ID NO: 56 - DNA, 20 bp, Artificial Sequence
  Nucleic acid sequence fragment
ctctcctccctccagctccc

SEQ ID NO: 57 - DNA, 20 bp, Artificial Sequence
  Nucleic acid sequence fragment
ctcttcgacatcctcctctc

SEQ ID NO: 58 - DNA, 21 bp, Artificial Sequence
  Nucleic acid sequence fragment
cctcgtcccttacttctcctg

SEQ ID NO: 59 - DNA, 24 bp, Artificial Sequence
  Nucleic acid sequence fragment
cctcataacttattgttgtaccag

SEQ ID NO: 60 - DNA, 19 bp, Artificial Sequence
  Nucleic acid sequence fragment
caagacgaccagagacagc

SEQ ID NO: 61 - DNA, 20 bp, Artificial Sequence
  Nucleic acid sequence fragment
gaactcgacaaatttgcctg

SEQ ID NO: 62 - DNA, 19 bp, Artificial Sequence
  Nucleic acid sequence fragment probe f
gggtgcaggtggaagaatg

SEQ ID NO: 63 - DNA, 20 bp, Artificial Sequence
  Nucleic acid sequence fragment probe r
ttgttgtaccagtacacacc

SEQ ID NO: 64 - DNA, 22 bp, Artificial Sequence
  Nucleic acid sequence fragment conserved f
gagggaggcgaaggagtgtatc

SEQ ID NO: 65 - DNA, 21 bp, Artificial Sequence
  Nucleic acid sequence fragment conserved r
caggagaagtaagggacgagg

SEQ ID NO: 66 - DNA, 22 bp, Artificial Sequence
  Nucleic acid sequence fragment degenerate f
attccttcctnctnctngtncc

SEQ ID NO: 67 - DNA, 20 bp, Artificial Sequence
  Nucleic acid sequence fragment degenerate r
gctaagtacaanggncancc

SEQ ID NO: 68 - DNA, 1162 bp, Brassica napus
ggccgtaggtcggtcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctgacctctctactgggcctgccaagggtgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcttccgttacgccgccgcgcagggagtggcctcgatggtctgcttctacggagtcccgcttctgattgtcaatggtttcctcgtgttgatcacttacttgcagcacacgcatccttccctgcctcactacgattcgtccgagtgggattggttgaggggagctttggctaccgttgacagagactacggaatcttgaacaaggtcttccacaatattaccgacacgcacgtggcgcatcatctgttctccacgatgccgcattatcacgcgatggaagctaccaaaggcgataaagccgatactggggagagtatttatcagttcgatgggacgcggtggttaggcgatgtgagggaggcgaggatgtatctattgtggaacggaacagcaggtgagaagaacgtgtgttctgtacaacatagtatgagaaatgatgatgtgaagaaccaagagatttgtcacgactttctctgcttctgggggccctttgtgga

SEQ ID NO: 69 - DNA, 70 bp, Brassica napus
gcagggagtggcctcgatggtctgcttctacggagtcccgcttctgattgtcaatggtttcctcgtgttg

SEQ ID NO: 70 - DNA, 70 bp, Brassica napus; identical to SEQ ID NO: 69

SEQ ID NO: 71 - DNA, 70 bp, Brassica napus
ccaaggagttgcctcgatggtctgcttctacggagttcctcttctgattgtcaacgggttcttagttttg

SEQ ID NO: 72 - DNA, 70 bp, Brassica napus; identical to SEQ ID NO: 71

SEQ ID NO: 73 - DNA, 70 bp, Brassica napus; identical to SEQ ID NO: 71

SEQ ID NO: 74 - DNA, 70 bp, Brassica napus; identical to SEQ ID NO: 71

SEQ ID NO: 75 - DNA, 70 bp, Brassica napus
gcgaggagtggcctcgatggtctgtgtctacggagttccgcttatgattgtcaactgtttcctcgtcttg

SEQ ID NO: 76 - DNA, 70 bp, Brassica napus; identical to SEQ ID NO: 75

SEQ ID NO: 77 - DNA, 70 bp, Brassica napus
gcgaggagtagcctctgtggtctgtgtctacggagttccgcttctaattgtcaactgtttcctcgtcttg

SEQ ID NO: 78 - DNA, 70 bp, Brassica napus; identical to SEQ ID NO: 77

SEQ ID NO: 79 - DNA, 70 bp, Brassica napus; identical to SEQ ID NO: 77

SEQ ID NO: 80 - DNA, 70 bp, Brassica napus; identical to SEQ ID NO: 77

SEQ ID NO: 81 - DNA, 70 bp, Brassica napus; identical to SEQ ID NO: 77

SEQ ID NO: 82 - DNA, 70 bp, Brassica napus; identical to SEQ ID NO: 77

SEQ ID NO: 83 - DNA, 67 bp, Brassica napus
catcatagcctcctgcttctactacgtcgccaccattactttcctcccccccctccacctccccccc

(19) United States
(12) Patent Application Publication
(10) Pub. No.: US 2014/0150132 A1
(43) Pub. Date: May 29, 2014

(54) Mutants

(71) Applicants: Ian Bancroft, Norwich (GB); Rachel Wells, Norwich (GB)

(72) Inventors: Ian Bancroft, Norwich (GB); Rachel Wells, Norwich (GB)

(73) Assignee: PLANT BIOSCIENCE LIMITED, Norwich, Norfolk (GB)

(21) Appl. No.: 14/002,464

(22) PCT Filed: Mar. 2, 2012
(86) PCT No.: PCT/GB12/50471

§ 371 Date: Nov. 22, 2013

(30) Foreign Application Priority Data
Mar. 2, 2011 (GB) 1103569.8

(51) Int. Cl. C12N 15/82; A01H 5/12; C12N 9/02

(52) U.S. Cl. CPC C12N 15/8247; C12N 9/0071; A01H 5/12
USPC 800/264; 536/23.2; 435/189; 800/306; 800/281

(57) ABSTRACT

Identification of new FAD2 mutants which result in plants with a more desirable oleic acid composition than in known plants. For the first time, this patent disclosure provides a complete characterization of the genome of a given germplasm of Brassica napus, and reports that there are, in fact, four FAD2 genes in any given genotype, and that in any given germplasm, one or more of the genes are active, thereby reducing the total percentage of oleic acid achievable in the total fatty acids produced in that germplasm. Armed with this knowledge, the inventors herein have produced a novel series of modifications in the genome of various Brassica napus germplasms and provide a germplasm with a compromised and/or totally inactive set of FAD2 genes.

### FIELD OF THE INVENTION

The present invention relates to new FAD2 mutants which result in plants with a desirable fatty acid composition, and particularly a desirable oleic acid content; although alternatively the present invention also allows a desirable polyunsaturated fatty acid content to be achieved. The present invention also relates to corresponding markers and their use in methods for the provision of new materials (plants, their progeny and seeds, and (bio)lubricants) comprising the desirable fatty acid composition.

### BACKGROUND OF THE INVENTION

The quality of edible and industrial oil derived from a plant is in part determined by its fatty acid composition. Both the type and amount of fatty acid unsaturation have implications for both dietary and industrials applications. Oils exhibiting reduced levels of polyunsaturated fatty acids (PUFAs) are associated with higher oxidative stability; the susceptibility of a fatty acid to oxidation being dependent on its degree of unsaturation. For example the rate of oxidation of linolenic acid (C18:3) is 100 times that of oleic acid (C18:1). The rate of oxidation of linoleic acid (C18:2) is also greater than that of oleic acid. Moreover, oleic acid has been associated with beneficial health effects, such as reduced cholesterol.

For many years, those skilled in this particular field have been exploring the production of high oleic acid plants. Many reports and patent documents exist in the art reporting various methods, means, compositions and plants in which, in particular, efforts have been made to silence, mutate, delete, inactivate or otherwise compromise the activity of the delta-12 oleate desaturase enzyme, known generally as FAD2.

For example, a non-exhaustive list of previously published patent documents in this field include the following: WO99/53050, disclosing, for example, at page 31-32, methods for using hairpin RNA molecules encoding portions of FAD2 (e.g. Genbank AF123460, AF12042841, L26296 or A65102) of oilseed rape (Brassica juncea, napes, rapa, oleracea, campestris, carinata) as well as corn, cotton, groundnut, sunflower, castor beans, flax, coconut, linseed, or soybean, to increase the oleic acid percentage of total fatty acid, with concomitant decrease in the percentage of the linolenic and linoleic acid percentage in total fatty acid in plants expressing such hairpin RNAs. See also, for example, the following patent disclosures by a wide and diverse series of patent applicants, relating to various efforts to achieve increased oleic acid content in a variety of different plants: U.S. Pat. No. 5,840,946 (Pioneer, “Vegetable Oil Extracted From Rapeseeds Having a Genetically Controlled Unusually High Oleic Acid Content”); WO2004/072259 (Dow Agrosciences, “Altered Fad2 and Fad3 Genes in Brassica and the Molecular Marker-Assisted Detection Thereof”); WO2007/107590 (Monsanto, “FAD-2 Mutants and High Oleic Plants”); WO2007/138444 (INRA, “Genetic Markers for High Oleic Acid Content in Plants”); U.S. Pat. No. 7,423,198 and U.S. Pat. No. 7,605,301 (Viterra, “High Oleic Acid Brassica juncea” and “Plant Fad2 Coding Sequence Balancing Fatty Acid Profiling in Edible Oils”, respectively).

The plethora of work and reports in this field notwithstanding, in Brassica napus, (oilseed rape) for example, there has been considerable confusion about exactly how many copies (or, as we shall term them, homologues) of the FAD2 desaturase are present and active in any given germplasm. In general, those skilled in the art have made reference to FAD2-1 and FAD2-2, which would lead those skilled in the art to conclude that there are but two homologues of the delta-12 oleate desaturase enzyme encoded by the B. napus genome.

Residual oleate desaturase activity has been attributed to other, compensating pathways, and those skilled in the art have not appreciated that there may be additional functional homologues of FAD2 in leading lines used for oleic acid production. Since any remaining FAD2 activity in a given plant will result in reduction in the oleic acid fraction and a corresponding increase of polyunsaturated fatty acids of the total fatty acids produced by that plant, there remains a need in the art to fully understand the genotype of oleic acid producing plants and seeds, to be able to optimally achieve a phenotype with maximal production of the this valuable commodity. The present invention meets that need.

On the other hand there is a growing body of evidence that the polyunsaturated fatty acids may be beneficial to health, for example in the areas of cardio-protection and brain development and tissue repair. There is also therefore a need in the art to fully understand the genotype of PUFA producing plants and seeds, to be able to optimally achieve a phenotype with maximal production of the this valuable commodity. The present invention meets that need.

Lubricants are a key element for industrial and transport applications. More than 95% of the market is currently satisfied by low-cost mineral oils. As 30% of lubricants inevitably end up in the ecosystem, natural based (bio) lubricants offer a potentially attractive alternative. However, there is a need to develop more economically sustainable means of producing such (bio)lubricants. Moreover degradation problems are associated with the use of a natural oil which is high in PUFAs. The present invention seeks to address these issues.

### SUMMARY OF THE INVENTION

For the first time, the present invention provides a complete survey of the genome for homologues of FAD2 and their characterization in a given germplasm of Brassica napus, and reports that there are, in fact, four FAD2 genes in any given genotype, present as homologous pairs of genes on sister chromosomes from the A and C genomes, namely BnaA.FAD2.a (chromosome A1), BnaC.FAD2.a (chromosome C1), BnaA.FAD2.b (chromosome A5), BnaC.FAD2.b (chromosome C5), and that in any given germplasm, one or more of the genes are active, thereby reducing the total percentage of oleic acid achievable in the total fatty acids produced in that germplasm. Examples of sequences of these four genes can be seen in FIGS. 12 and 13 and these also form part of the present invention. FIG. 12 shows ˜97% sequence similarity. The sequences are difficult to separate via homologue-specific PCR due to this high level of sequence conservation, hence providing details of each of these sequences is an important contribution to the art. Such sequences form part of the invention.

The inventors have produced a novel series of modifications in the genome of various Brassica napus germplasms and provide a germplasm with a compromised and/or totally inactive set of FAD2 genes. A wide variety of new mutations in the FAD2 homologue BnaC.FAD2.b are provided which compromise or abolish activity of the enzyme encoded by that gene. These mutations include, but are not limited to: conversion of a codon encoding an amino acid to a stop codon; conversion of a codon to encode an alternative amino acid; and the like. As exemplified in FIG. 6, a wide number of single nucleotide modifications, deletions, insertions or the like are available to achieve these objectives, including, but not limited to, the specific nucleotide changes (and the resultant amino acid changes) and combinations thereof. By producing germplasm comprising any one or a combination of these mutations in BnaC.FAD2.b of B. napus, it is possible, depending on the starting genotype of the B. napus variety used, to produce a B. napus germplasm in which there is no active FAD2 enzyme activity at all. Thus, for example, starting with variety Cabriolet, which we have found has one active homologue of FAD2, namely BnaC.FAD2.b, while the other three homologues are not active, we have been able to produce a new variety of B. napus which has no active FAD2 whatsoever. Those skilled in the art will appreciate, therefore, that any given germplasm may be utilized as starting material and the work disclosed herein reproduced to compromise the activity of the homologue equivalent in that variety to BnaC.FAD2.b of B. napus, and this will produce a new variety in which that homologue is compromised in activity. Used in combination with known mutations or varieties in which any of the other active homologues are likewise inactivated or compromised, new varieties are producible which have some, little or no delta-12 oleate desaturase activity.

Accordingly, it is a first object of this invention to provide a novel germplasm for the efficient production of a desirable fatty acid composition in plants cultivated for this purpose. The desirable fatty acid composition is one which has a relatively high level of monounsaturated fatty acid, in more detail a relatively high level of oleic acid. Preferably the desirable fatty acid composition has a relatively low level of PUFA, in more detail a relatively low level of linoleic and/or linolenic fatty acid.

In a second aspect of the present invention, however, the desirable fatty acid composition is one which has a relatively high level of polyunsaturated fatty acid, in more detail a relatively high level of linoleic and/or linoleic acid. Preferably the desirable fatty acid composition has a relatively low level of monounsaturated fatty acid, in more detail a relatively low level of oleic.

It is a further object of this invention to provide plants and particularly a Brassica napus with a totally inactivated FAD2.

It is a further object of this invention to provide methods for producing the desirable fatty acid content in a plant and to provide tools, such as primers and genetic markers, to enable a skilled worker to select such a plant.

### STATEMENTS OF THE INVENTION

According to the first aspect of the present invention, there is provided a mutated nucleic acid sequence of a Brassica FAD2-encoding nucleic acid sequence, wherein said nucleic acid sequence has one or more of the mutations shown in FIG. 6 in relation to the BnaC.FAD2.b gene, or a homologous nucleic acid sequence derived from said mutated nucleic acid sequence by substitution, insertion or deletion of at least one nucleotide, and presenting at least 90% homology with said mutated nucleic acid sequence, provided that said homologous nucleic acid sequence does not encode an active FAD2 enzyme, or a hybridizing nucleic acid sequence which hybridizes under stringent conditions to said mutated nucleic acid sequence, provided that the complementary sequence of said hybridizing nucleic acid sequence does not encode an active FAD2 enzyme, or a complementary sequence of one of said mutated, homologous or hybridizing nucleic acid sequences.

The mutation may be one of the following mutations: 224C to T (75 Pro to Leu), 241C to T (81 Leu to Phe), 257G to A (86 Typ to Stop), 258G to A (86 Trp to Stop), 270G to A (90 Trp to Stop), 284G to A (95 Cys to Tyr), 296G to A (99 Gly to Asp), 310G to A (104 Ala to Thr), 316G to A (106 Glu to Lys), 322G to A (108 Gly to Ser), 328C to T (110 His to Tyr), 350G to A (117 Trp to Stop), 355G to A (119 Asp to Asn), 367G to A (123 Gly to Ser), 388C to T (130 Leu to Phe), 421C to T (141 His to Tyr), 425G to A (143 Arg to Gln), 428G to A (143 Arg to His), 437C to T (146 Ser to Phe), 458G to AG (153 Arg to Lys), 543G to A (181 Met to Ile), 566G to A (189 Gly to Asp), 570G to A (190 Trp to Stop), 598G to A (200 Gly to Arg), 616G to A (206 Gly to Tyr), 617G to A (206 Gly to Asp), 623C to T (208 Ala to Val), 637C to T (213 Pro to Ser), 642C to A (214 Asn to Lys), 643G to A (215 Ala to Thr), 704C to T (235 Ala to Val), 710G to A (237 Cys to Tyr), 716G to A (239 Gly to Asp), 743G to A (248 Gly to Glu), 776C to T (259 Pro to Leu), 778C to T (260 Leu to Phe), 890C to T (297 Thr to Ile).

In one embodiment, in relation to the BnaA.FAD2.b gene, the mutation is not one of the mutations shown in FIG. 15, i.e. is not 662G to A (221 Arg to His) or 737C to T (246 Ala to Val).

According to the first aspect of the present invention there is also provided a mutated nucleic acid sequence of a Brassica FAD2-encoding nucleic acid sequence, wherein said nucleic acid sequence encodes a FAD2 enzyme in which amino acid position 241 and/or 246 are changed in relation to the FAD2 enzyme encoded by BnaC.FAD2.b gene, or

a homologous nucleic acid sequence derived from said mutated nucleic acid sequence by substitution, insertion or deletion of at least one nucleotide, and presenting at least 90% homology with said mutated nucleic acid sequence, provided that said homologous nucleic acid sequence does not encode an active FAD2 enzyme, or a hybridizing nucleic acid sequence which hybridizes under stringent conditions to said mutated nucleic acid sequence, provided that the complementary sequence of said hybridizing nucleic acid sequence does not encode an active FAD2 enzyme, or
a complementary sequence of one of said mutated, homologous or hybridizing nucleic acid sequences.

Also according to the first aspect of the present invention there is provided a mutated nucleic acid sequence of a Brassica FAD2-encoding nucleic acid sequence, wherein said nucleic acid sequence has a 1 bp deletion at coding base position 215 in relation to the BnaA.FAD2.b gene, or

a homologous nucleic acid sequence derived from said mutated nucleic acid sequence by substitution, insertion or deletion of at least one nucleotide, and presenting at least 90% homology with said mutated nucleic acid sequence, provided that said homologous nucleic acid sequence does not encode an active FAD2 enzyme, or a hybridizing nucleic acid sequence which hybridizes under stringent conditions to said mutated nucleic acid sequence, provided that the complementary sequence of said hybridizing nucleic acid sequence does not encode an active FAD2 enzyme, or
a complementary sequence of one of said mutated, homologous or hybridizing nucleic acid sequences.

The mutations of the first aspect of the present invention are associated with the first desirable fatty acid composition in which the level of monounsaturated fatty acid, and preferably oleic acid, is increased.

On the other hand according to a second aspect of the present invention, there is provided a mutated nucleic acid sequence of a Brassica FAD2-encoding nucleic acid sequence, wherein said nucleic acid sequence has one or more of the mutations shown in FIG. 15, i.e. preferably 662G to A (221 Arg to His) and/or 737C to T (246 Ala to Val) in relation to the BnaC.FAD2.b gene, or a homologous nucleic acid sequence derived from said mutated nucleic acid sequence by substitution, insertion or deletion of at least one nucleotide, and presenting at least 90% homology with said mutated nucleic acid sequence, provided that said homologous nucleic acid sequence encodes an active FAD2 enzyme, or a hybridizing nucleic acid sequence which hybridizes under stringent conditions to said mutated nucleic acid sequence, provided that the complementary sequence of said hybridizing nucleic acid sequence encodes an active FAD2 enzyme, or

a complementary sequence of one of said mutated, homologous or hybridizing nucleic acid sequences.

The homology level may be at or at least 95%, 97%, 98% or 99% identity.

The mutations of the second aspect of the present invention are associated with the second desirable fatty acid composition in which the level of polyunsaturated fatty acid, and preferably linoleic and/or linolenic acid, is increased.

In one embodiment, the mutations are not those of the first aspect of the invention.

FAD2 as referred to herein means delta-12 oleate desaturase enzyme.

As used herein, the phrase “nucleic acid” refers to any physical string of monomer units that can be corresponded to a string of nucleotides, including a polymer of nucleotides (e.g., a typical DNA, cDNA or RNA polymer), modified oligonucleotides (e.g., oligonucleotides comprising bases that are not typical to biological RNA or DNA, such as 2′-O-methylated oligonucleotides), and the like. In some embodiments, a nucleic acid can be single-stranded, double-stranded, multi-stranded, or combinations thereof. Unless otherwise indicated, a particular nucleic acid sequence of the presently disclosed subject matter optionally comprises or encodes complementary sequences, in addition to any sequence explicitly indicated.

Mutations as referred to herein include addition, deletion, substitution, modification, replacement and/or variation of at least one nucleotide. The mutated nucleic acid sequences may be truncated. Preferably the mutation will cause addition, deletion, substitution modification replacement and/or variation of at least one amino acid residue present in the FAD2 encoded by the mutated nucleic acid sequence. For example, mutations in coding sequences may be made so as to introduce substitutions within functional motifs in FAD2. Mutations in the nucleic acid sequences may cause the production of a truncated FAD2 amino acid sequence.

A mutation may cause inactivation of the mutated nucleic acid sequence or inactivation of the FAD2 enzyme encoded by said mutated nucleic acid sequence.

Mutations may be introduced using synthetic oligonucleotides. These oligonucleotides contain nucleotide sequences flanking the desired mutation sites.

A suitable method is disclosed in Morinaga et al., (Biotechnology (1984) 2, p646-649). Another method of introducing mutations into enzyme-encoding nucleotide sequences is described in Nelson and Long (Analytical Biochemistry (1989), 180, p 147-151).

The present invention also encompasses sequences that are complementary to the nucleic acid sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

In one embodiment, the present invention encompasses sequences that are complementary to sequences that are capable of hybridising under stringent conditions (e.g. 50° C. and 0.2×SSC {1×SSC=0.15 M NaCl, 0.015 M Na₃citrate pH 7.0}) to the nucleotide sequences presented herein.

More preferably, the present invention encompasses sequences that are complementary to sequences that are capable of hybridising under high stringent conditions (e.g. 65° C. and 0.1×SSC {1×SSC=0.15 M NaCl, 0.015 M Na₃citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

Also included within the scope of the present invention are polynucleotide sequences that are capable of hybridising to the nucleotide sequences presented herein under conditions of intermediate to maximal stringency.

In a preferred aspect, the present invention covers nucleotide sequences that can hybridise to the nucleotide sequence of the present invention, or the complement thereof, under stringent conditions (e.g. 50° C. and 0.2×SSC).

In a more preferred aspect, the present invention covers nucleotide sequences that can hybridise to the nucleotide sequence of the present invention, or the complement thereof, under high stringent conditions (e.g. 65° C. and 0.1×SSC).

The mutations have been described with reference to a reference sequence but it will be appreciated that the mutations may be at a corresponding position in another sequence. Thus it will be appreciated that the nucleotide referred to may differ in the indicated number but still have similar neighbouring nucleotides.

It will be appreciated that mutated nucleic acid sequences of the present invention can be introduced into plants or parts thereof using routine techniques. Thus, the present invention also encompasses vectors, host cells, plants and parts of plants comprising the mutated nucleic acid sequences. The introduction of the nucleic acid sequences into plants or parts thereof is more particularly relevant to the second aspect of the present invention where one is in general seeking to increase the FAD2 enzymatic activity to increase the level of polyunsaturated fatty acid.

According to a further aspect of the present invention, there is provided a fragment of a mutated nucleic acid sequence of the invention which comprises a specified mutation. Preferred fragments include fragments having at least 5, 10, 15, 20, 30, 40, 50 or 100 contiguous nucleic acid from a mutated nucleic acid sequence of the present invention or fragments having at least 5, 10, 15, 20, 30, 40, 50 or 100 contiguous nucleic acids truncated or deleted from a mutated nucleic acid sequences of the present invention. A fragment may be part of a longer nucleic acid sequence. In one embodiment the fragment comprises at least 5 contiguous wild-type bases on either side of the mutation according to the present invention.

Preferably the fragment is identical in sequence to a portion of a modified BnaC.FAD2.b gene.

Preferably the fragment is selected from the group consisting of

SEQ ID. 52:
GTCTCCTCCCTCCAAAAAGT
SEQ ID. 53:
GTGTCTCCTCCCTCCAAA
SEQ ID. 54:
CTACAGAAACAAACATGGGC
SEQ ID. 55:
GTCTCTCCTCCCTCCAGC
SEQ ID. 56:
CTCTCCTCCCTCCAGCTCCC
SEQ ID. 57:
CTCTTCGACATCCTCCTCTC
SEQ ID. 58:
CCTCGTCCCTTACTTCTCCTG
SEQ ID. 59:
CCTCATAACTTATTGTTGTACCAG
SEQ ID. 60:
CAAGACGACCAGAGACAGC
SEQ ID. 61:
GAACTCGACAAATTTGCCTG
SEQ ID. 62:
GGGTGCAGGTGGAAGAATG probe f,
SEQ ID. 63:
TTGTTGTACCAGTACACACC probe r,
SEQ ID. 64:
conserved f GAGGGAGGCGAAGGAGTGTATC,
SEQ ID. 65:
conserved r CAGGAGAAGTAAGGGACGAGG,
SEQ ID. 66:
degenerate f ATTCCTTCCTNCTNCTNGTNCC,
SEQ ID. 67:
degenerate r GCTAAGTACAANGGNCANCC.

The nucleic acids of the present invention, including the fragments thereof, can usefully be used as markers, primers or probes. They can usefully be employed in combination.

The nucleic acids of the present invention can thus be used as markers in the identification of genes encoding desirable phenotypic traits in Brassica. The present invention thus allows a skilled worker to advantageously reliably breed for the markers of the present invention and hence reliably breed for an economically important fatty acid composition.

In one aspect, the invention provides use of a nucleic acid sequence to guide site-specific mutation in a regulatory region of a FAD2 gene. For example, sequence from the upstream region of the FAD2 gene may be used to guide site-specific mutations in the FAD2 regulatory region such as the TATA box in order to down-regulate expression of the FAD2 gene. Similarly, sequence from the upstream region of the FAD2 gene could be used to guide site-specific mutations in the FAD2 regulatory region to down-regulate expression of the FAD2 gene. This may be done in vitro or in vivo.

In one aspect, the invention provides amplification primers or probes that may be used to identify FAD2 nucleic acid sequences of the invention, or the region upstream from the genes from other nucleic acid sequences. For example, primers or probes may be synthesised that are complementary to portions of the naturally occurring oleate desaturase coding sequence. Selected primers may be capable of distinguishing plants having high oleic acid content from plants having low oleic acid content or vice versa.

In another aspect of the invention, selective hybridisation and amplification, using FAD2 locus-specific probes and primer pairs of the invention, may be used to generate an amplification pattern that may contribute to a collection of DNA fingerprints to identify the FAD2 genotype of a germ-plasm. FAD2 probes may for example include primers or probes synthesised from complementary portions of the naturally occurring coding sequences of the oleate desaturase FAD2 genes and from complementary portions upstream of the FAD2 genes.

According to the first aspect the invention comprises a method of selecting plants having a high oleic acid content by utilizing PCR primers to selectively amplify a desired gene. This method may be used, for example, to ensure the selected progeny carry a desired coding sequence conferring a high oleic acid oil phenotype.

According to the second aspect the invention comprises a method of selecting plants having a high PUFA content by utilizing PCR primers to selectively amplify a desired gene. This method may be used, for example, to ensure the selected progeny carry a desired coding sequence conferring a high PUFA oil phenotype.

In accordance with an embodiment of the method, seedlings of a first segregating backcross population, may be subjected to a PCR analysis to detect the mutant FAD2 nucleic acid, and the selected plants backcrossed again to a recurrent parental line. The backcrossing and PCR analysis of the first seedling population may, for example, proceed through at least two more cycles to create a third segregating backcross seedling population, which may be self-pollinated to create a third seedling population. The third seedling population may be subjected to PCR analysis for the mutant nucleic acid, and homozygotes may be selected for further pedigree breeding, such as breeding of an elite, high oleic acid content strain.

In another aspect there is also provided a method of selecting plants having a desired fatty acid content comprising the following steps:
crossing a plant or part thereof of the present invention with a parent plant;
selecting from the progeny plants or parts thereof those comprising at least one sequence of the present invention.

Crossing can be carried out using methods well known to skilled workers.

In one embodiment the selection is carried out using a nucleic acid of the present invention, in particular a fragment thereof.

The present invention also relates to a plant obtainable using the methods of the present invention.

The present invention also relates to a kit comprising a nucleic acid sequence or fragment of the present invention, particularly for the selection of plants with a desirable fatty acid profile.

In another embodiment of the first aspect the present invention relates to a method of providing a plant having increased monounsaturated fatty acid content, particularly oleic acid content, comprising the steps of:
inactivating at least one FAD2-encoding nucleic acid sequence of the present invention in a plant or part thereof,
selecting a plant or part thereof or regenerated plant comprising said inactivation.

In a preferred method of producing a plant which is subject to the inactivation is pre-selected such that it contains only one active FAD2 gene or enzyme.

The present invention enables a skilled worker to provide a new plant in which all nucleic acid sequences encoding FAD2 or the enzyme are inactivated.

Thus in one preferred embodiment of the first aspect the present invention relates to a method of producing a plant or a part thereof, said method selected from the group consisting of (a) silencing each of the homologues of the gene encoding that enzyme (b) producing mutants in the genome to reduce or destroy the activity of any encoded enzyme (c) selecting a variety of Brassica in which only one active FAD2 gene exists, mutating that gene and producing a new variety in which all genes encoding oleate-12 desaturase are modified as compared with wild-type of that variety such that any oleate desaturase protein encoded is compromised in activity or is devoid of activity and (d) marker assisted selection of crosses between particular varieties to produce a variety which encodes little or no active FAD2 enzymatic activity.

In another embodiment of the first aspect the present invention relates to a method of providing a plant having increased PUFA content, particularly linoleic acid and/or linolenic fatty acid content, comprising the steps of:
activating at least one FAD2-encoding nucleic acid sequence of the present invention in a plant or part thereof,
selecting a plant or part thereof or regenerated plant comprising said activation.

Thus in one preferred embodiment of the second aspect the present invention relates to a method of producing a plant or a part thereof, said method selected from the group consisting of (a) introducing or upregulating expression of more or more of the homologues of the gene encoding that enzyme (b) producing mutants in the genome to increase the activity of any encoded enzyme (c) selecting a variety of Brassica in which a FAD2 gene exists, mutating that gene and producing a new variety in which a gene and preferably all genes encoding oleate-12 desaturase are modified as compared with wild-type of that variety such that any oleate desaturase protein encoded is improved or gains activity and (d) marker assisted selection of crosses between particular varieties to produce a variety which encodes active FAD2 enzymatic activity.

By activated we include that the protein has FAD2 enzymatic activity. By activating we include that the protein has increased FAD2 activity compared to the parent protein or the plant or part thereof.

According to another aspect of the present invention there is provided a protein or polypeptide comprising an amino acid sequence encoded by a mutated nucleic acid sequences of the present invention or fragment thereof.

The protein or polypeptide may be a fragment of FAD2.

The mutated nucleic acid sequences and fragments thereof, as well as proteins or polypeptides encoded by said mutated nucleic acid sequences and fragments may be used to produce the desirable fatty acid composition of a plant or part thereof.

According to further aspect of the present invention there is provided a plant or part thereof in which more than two FAD2-encoding nucleic acid sequences or the corresponding FAD2 enzymes are inactivated such that the enzymatic activity is reduced compared to the non-inactivated version.

In one embodiment, the plant or part thereof has more than three FAD2-encoding nucleic acid sequences or the corresponding FAD2 enzymes are inactivated.

In another embodiment, the plant or part thereof has all of the FAD2 enzymes or the nucleic acid sequences encoding therefore are inactivated.

Plants or parts thereof according to the invention are provided in which the nucleic acid sequence is one or more of the mutated nucleic acid sequences of the invention or fragments thereof.

It will be appreciated that the aspects of the present invention which are based on the technical teaching of the surprising number of FAD2 genes are generally applicable, particularly for all oil producing crops. Such crops include sunflower, soybean, palm, corn etc., but are preferably Brassica species. Brassica is particularly preferred where the present invention involves the sequences and mutations of the present invention.

The Brassica plant of part thereof according to the invention may be selected from the group consisting of Brassica juncea, napus, rapa, oleracea, campestris, carinata.

The Brassica plant of part thereof according to the invention may be Brassica napus. In one embodiment, the Brassica napus plant or part thereof is derivable from the variety Cabriolet or Tapidor.

In another embodiment there is provided a Brassica napus plant or part thereof in which each of the four FAD2-encoding nucleic acid sequences or the corresponding FAD2 enzymes are inactivated. The nucleic acid sequences may be selected from the group consisting of:

### BnaA.FAD2.a

### BnaC.FAD2.a

### BnaA.FAD2.b or

### BnaC.FAD2.b.

The FAD2-encoding nucleic acid sequences or the corresponding FAD2 enzymes may be inactivated to increase the 18:1 (oleic acid) content of the Brassica plant or part thereof.

The FAD2-encoding nucleic acid sequences or the corresponding FAD2 enzymes may be inactivated to reduce the polyunsaturated fatty acid (PUFA) content of the Brassica plant or part thereof. Preferably, the 18:2 and 18:3 fatty acid content of the Brassica plant or part thereof is reduced.

As used herein in general terms “inactivation” refers to a nucleic acid which does not encode a functional FAD2 protein or it refers to a non-functional FAD2 protein and which FAD2 protein has an activity which is lower than that of the corresponding FAD2 protein, usually the natural FAD2 protein, measured in the same conditions, preferably the FAD2 protein has no enzymatic activity.

Inactivated as referred to herein refers to the outcome of methods which include FAD2 gene silencing and the reduction or elimination of expression of a nucleic acid sequence that encodes FAD2. By elimination of expression, it is meant herein that a functional amino acid sequence encoded by the nucleic acid sequence is not produced at a detectable level. By a reduction of expression, it is meant herein that a functional amino acid sequence encoded by the nucleic acid sequence is produced at a level that is reduced compared to when a FAD2 encoding sequence is not inactivated.

Inactivation may include the reduction or elimination of transcription of a nucleic acid sequence that encodes FAD2. By elimination of transcription it is meant herein that the mRNA sequence encoded by the nucleic acid sequence is not transcribed at detectable levels. By reduction of transcription it is meant herein that the mRNA sequence encoded by the nucleic acid sequence is transcribed at levels that are reduced compared to when a FAD2 encoding sequence is not inactivated.

Inactivation may include the reduction or elimination of translation of a nucleic acid sequence that encodes FAD2. By elimination of translation it is meant herein that the mRNA sequence encoded by the nucleic acid sequence is not translated at detectable levels. By reduction of translation it is meant herein that the mRNA sequence encoded by the nucleic acid sequence is translated at levels that are reduced compared to when a FAD2 encoding sequence is not inactivated.

Inactivation may include the reduction or elimination of FAD2 enzyme activity. By elimination of FAD2 enzyme activity it is meant herein that the enzyme has no detectable activity. By reduction of FAD2 enzyme activity it is meant herein that the enzyme activity is reduced compared to when FAD2 is not inactivated. A reduction in FAD2 enzyme activity may be measured by measuring the corresponding increase in for example 18:1 (oleic acid) content in a plant or part thereof or another desirable fatty acid composition trait.

Inactivation may also include the production of a truncated amino acid sequence from a nucleic acid sequence that encodes FAD2. By production of a truncated amino acid sequence it is meant herein that the amino acid sequence encoded by the nucleic acid sequence is missing one or more amino acids of the functional amino acid sequence encoded by a wild type nucleic acid sequence. In addition, inactivation may include the production of a variant FAD2 amino acid sequence. By production of a variant amino acid sequence it is meant herein that the amino acid sequence has one or more amino acids that are different from the amino acid sequence encoded by a FAD2 nucleic acid sequence that has not been inactivated.

In general, unless otherwise specified, when referring to a “plant” it is intended to cover a plant at any stage of development, including cells and seeds and germplasm.

A plant “part thereof” includes leaves, stems, roots, flowers or flower parts, fruits, pollen, egg cells, zygotes, seeds, cuttings, cell or tissue cultures, or any other part or product of a plant.

Advantageously the plants or parts of the present invention have the desired fatty acid composition of the first aspect of the present invention, i.e. an increased monounsaturated fatty acid content, particularly an increased oleic acid content and/or a reduced PUFA character, particularly a reduced linolenic and/or linoleic fatty acid content, as compared to otherwise genetically identical plants, i.e. plant which are identical except for the presence of the mutation, and/or to corresponding wild-type plants.

Advantageously the plants or parts of the present invention have the desired fatty acid composition of the second aspect of the present invention, i.e. an increased polyunsaturated fatty acid content, particularly an increased linoleic acid content and/or linolenic acid content, and/or a reduced monounsaturated fatty acid character, particularly a reduced oleic fatty acid content, as compared to otherwise genetically identical plants, i.e. plant which are identical except for the presence of the mutation, and/or to corresponding wild-type plants.

Regeneration of plants from the plants or part thereof of the present invention can be carried out using methods well known to a skilled worker. Thus, the present invention also extends to the progeny of the plants, preferably such progeny also have the desirable fatty acid composition.

A plant or part according to the invention may be a cell or seed.

In one embodiment of the first aspect of the present invention there is provided a seed the fatty acid composition of which is greater, by at least 5%, than 74.5%, 74.7%, 75%, 76%, 77%, 78%, 79%, 80%, 81%, 82%, 83%, 84% or 85% oleic acid and which preferably also has an 18:2 and/or 18:3 fatty acid fraction which is less by at least 5% than 16.1%, 15.9%, 15.7% down to around 6%.

In one embodiment of the first aspect there is provided a seed of a plant that has at least one FAD2-encoding nucleic acid sequence or a FAD2 enzyme inactivated, wherein the oleic acid content of the seed is greater, by at least 5, 10, 15, 20, 25, 30, 50, 75, 100%, than the seed of a plant which does not have a FAD2-encoding nucleic acid sequence or a FAD2 enzyme inactivated.

In one embodiment of the first aspect there is provided a seed of a plant that has at least one FAD2-encoding nucleic acid sequence or a FAD2 enzyme inactivated, wherein the PUFA content and/or 18:2 and/or 18:3 fatty acid content of the seed is less by at least 5, 10, 15, 20, 25, 30, 50, 75, 100% than the seed of a plant which does not have a FAD2-encoding nucleic acid sequence or a FAD2 enzyme inactivated.

In one embodiment of the first aspect there is provided a seed of a plant that has at least one FAD2-encoding nucleic acid sequence or a FAD2 enzyme inactivated, wherein the oleic acid content of the seed is greater, by at least 5, 10, 15, 20, 25, 30, 50, 75, 100%, than the seed of a plant which does not have a FAD2-encoding nucleic acid sequence or a FAD2 enzyme inactivated, and wherein the PUFA content and/or 18:2 and/or 18:3 fatty acid content of the seed is less by at least 5, 10, 15, 20, 25, 30, 50, 75, 100% than the seed of a plant which does not have a FAD2-encoding nucleic acid sequence or a FAD2 enzyme inactivated.

In one embodiment of the second aspect of the present invention there is provided a seed the fatty acid composition of which is less, by at least 5%, than 74.5%, 74.7% or 75% oleic acid and which preferably also has an 18:2 and/or 18:3 fatty acid fraction which is greater by at least 5% than 16.1%, 15.9%, 15.7%. It could be envisaged that there is provided a seed the fatty acid composition of which is greater, by at least 5%, than 74.5%, 74.7%, 75%, 76%, 77%, 78%, 79%, 80%, 81%, 82%, 83%, 84% or 85% PUFA (preferably 18:2 and 18:3) and which preferably also has an 18:1 fatty acid fraction which is less by at least 5% than 16.1%, 15.9%, 15.7% down to about 6%.

In one embodiment of the second aspect there is provided a seed of a plant that has at least one FAD2-encoding nucleic acid sequence or a FAD2 enzyme, wherein the linoleic acid content and/or linolenic acid content of the seed is greater, by at least 5, 10, 15, 20, 25, 30, 50, 75, 100%, than the seed of a plant which does not have a FAD2-encoding nucleic acid sequence or has an FAD2 enzyme inactivated.

In one embodiment of the second aspect there is provided a seed of a plant that has at least one FAD2-encoding nucleic acid sequence or a FAD2 enzyme, wherein the PUFA content and/or 18:2 and/or 18:3 fatty acid content of the seed is increased by at least 5, 10, 15, 20, 25, 30, 50, 75, 100% than the seed of a plant which does not have a FAD2-encoding nucleic acid sequence or has an FAD2 enzyme inactivated.

In one embodiment of the second aspect there is provided a seed of a plant that has at least one FAD2-encoding nucleic acid sequence or a FAD2 enzyme, wherein the linoleic acid and/or linolenic acid content of the seed is greater, by at least 5, 10, 15, 20, 25, 30, 50, 75, 100%, than the seed of a plant which does not have a FAD2-encoding nucleic acid sequence or has an FAD2 enzyme inactivated, and

wherein the monounsaturated fatty acid content and/or 18:1 fatty acid content of the seed is less by at least 5, 10, 15, 20, 25, 30, 50, 75, 100% than the seed of a plant which does not have a FAD2-encoding nucleic acid sequence or has an FAD2 enzyme inactivated.

The seeds of the present invention are preferably Brassica seeds. These seeds according to the present invention may be from a Brassica plant selected from the group consisting of Brassica juncea, napus, rapa, oleracea, campestris, carinata.

A Brassica seed according to the present invention may be a Brassica napus seed.

According to a further aspect of the present invention there is provided fatty acid, preferably comprising oleic acid, produced from the seed or the Brassica plant according to the present invention. The present invention extends to a purified fatty acid composition as well as the directly extracted composition, and to the individual fatty acids present in the fatty acid composition.

According to a further aspect of the present invention there is provided a (bio)lubricant containing the fatty acid composition of the present invention.

Other objects and advantages of this invention will be appreciated by those skilled in the art from a review of the complete disclosure provided herein and the appended claims.

### BRIEF DESCRIPTION OF THE DRAWINGS

FIG. 1—PCR primer positions on the various Cabriolet FAD2 homologues.

FIG. 2—Agarose gel showing BnaC.FAD2.b specific PCR used for mutation load screen on individuals from the population.

FIG. 3—Agarose gel showing homologue specific PCR on Tapidor (T) and Cabriolet (C), a) BnaA.FAD2.b, b) BnaC.FAD2.a, no amplification is seen in Cabriolet which does not possess this locus, c) and d) BnaA.FAD2.a generic PCR amplifies both the chromosome A1 and C1 homologues of the gene, e) BnaA.FAD2.a

FIG. 4 shows the alignment of Tapidor and Cabriolet sequences against the different homologues. Note amplification of Cabriolet BnaA.FAD2.a with BnaC.FAD2.a primers under touchdown condition (trace highlighted).

FIG. 5 mixed amplicon of Cabriolet BnaC.FAD2.b and BnaA.FAD2.b caused by the 1 bp deletion occurring in BnaA.FAD2.b at base 160

FIG. 6 shows the oleic acid (18:1) fraction compared to other fatty amino acid percentages in wild type and mutants produced according to this invention after the first year of trialling

FIG. 7—CLUSTAL 2.0.12 multiple sequence alignment Tapidor genomic

FIG. 8—CLUSTAL 2.0.12 multiple sequence alignment Tapidor protein

FIG. 9—CLUSTAL 2.0.12 multiple sequence alignment between the nucleotide sequences of Tapidor and Cabriolet

FIG. 10—alignment of Cabriolet and Tapidor Amino Acid sequences

FIG. 11—Mutants aligned against BnaA.FAD2.b from Cabriolet and Tapidor—B=poor quality bases S=position of mutation

FIG. 12—CLUSTAL 2.1 multiple sequence alignment—Coding region alignment of A5 and C5 FAD2 homologues, BnaA.FAD2.b and BnaC.FAD2.b

FIG. 13—Coding region alignment of A1 and C1 FAD2 homologues, BnaA.FAD2.a and BnaC.FAD2.a

FIG. 14—Protein alignment of the three functional FAD2 proteins from Tapidor, BnaC.FAD2.b, BnaA.FAD2.b and BnaC.FAD2.a

FIG. 15—Mutations decreasing oleic acid content and increasing PUFA content

### DETAILED DISCLOSURE OF THE PREFERRED EMBODIMENTS OF THE INVENTION

The practice of the present invention will employ, unless otherwise indicated, conventional techniques of chemistry, molecular biology, microbiology, recombinant DNA and plant biology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Col d Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and E. M. Shevach and W. Strober, 1992. Each of these general texts is herein incorporated by reference.

In the present patent disclosure, we characterize the complete genotype of a Brassica napus germplasm, we identify four FAD2 homologues, and we provide a series of mutants in which all FAD2 homologues are inactivated. As a result, we provide certain isolates which exhibit production of high oleic acid in the rapeseed product, with, in certain instances, production in excess of 90% of the total fatty acid being oleic acid.

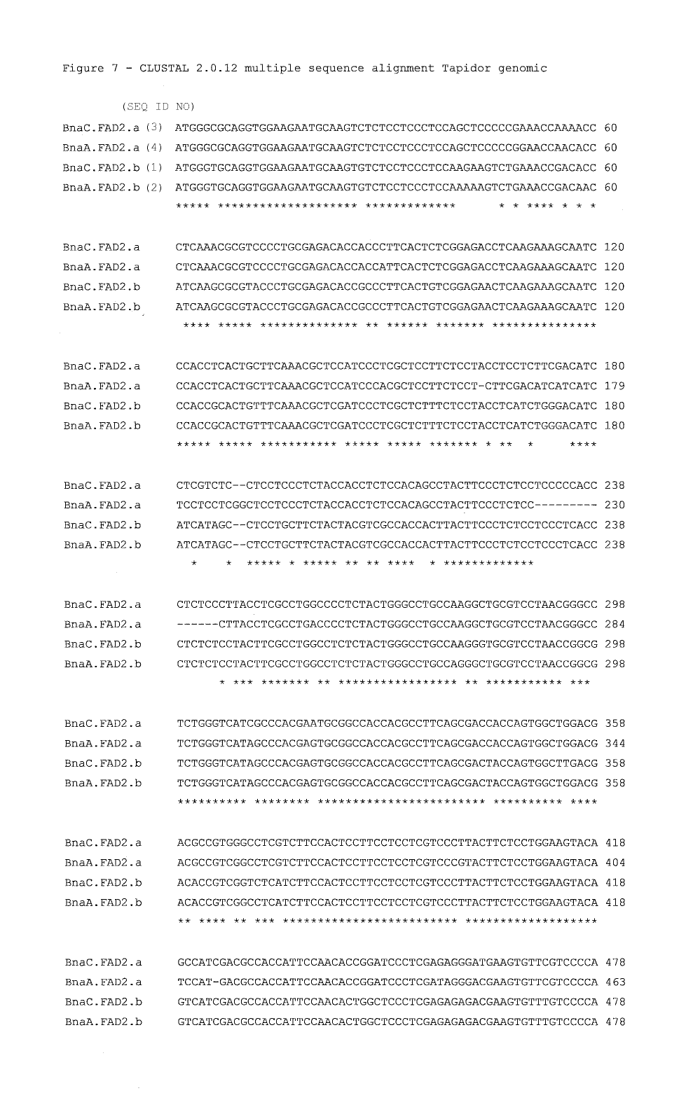

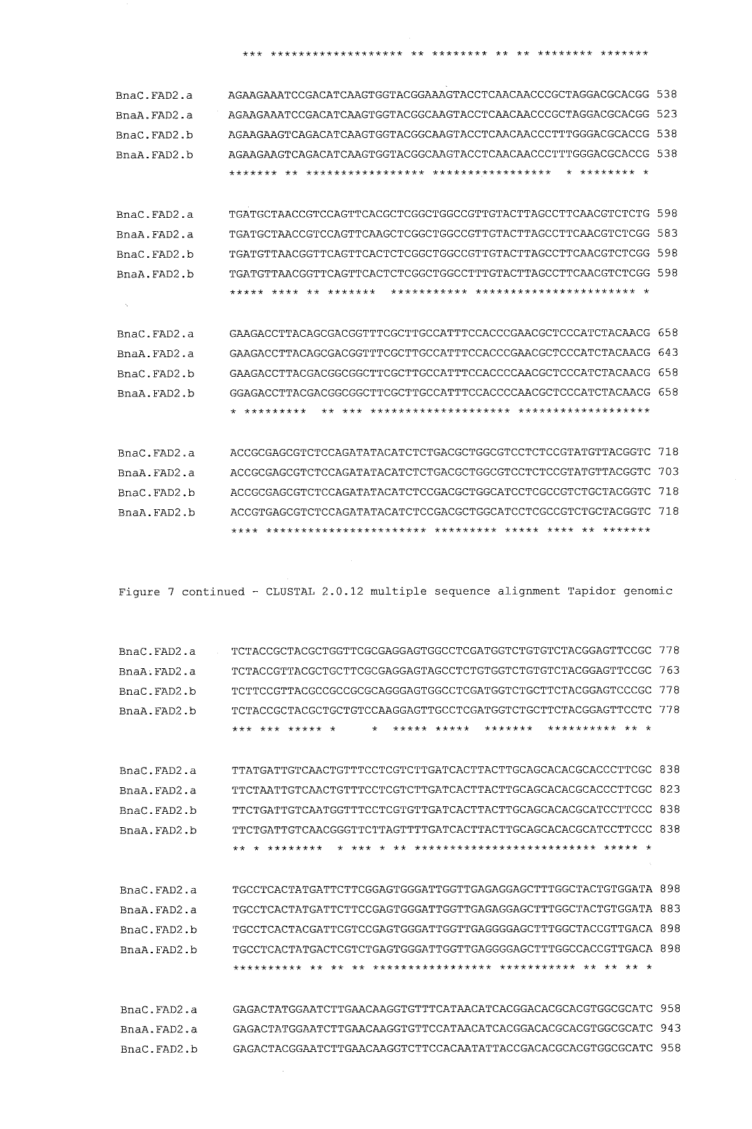

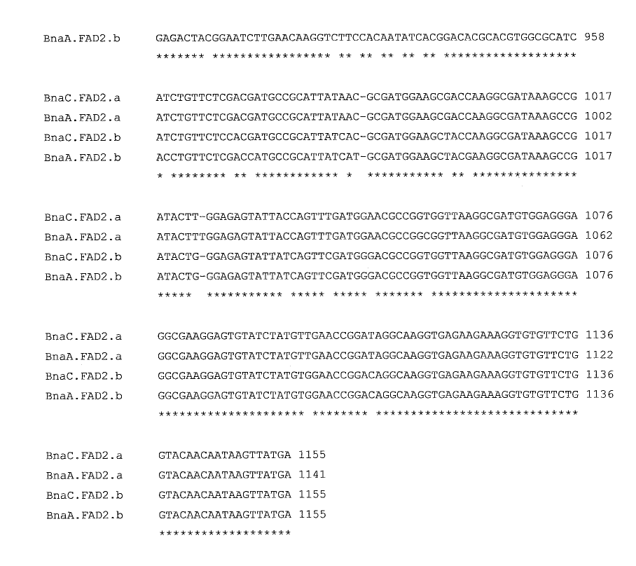

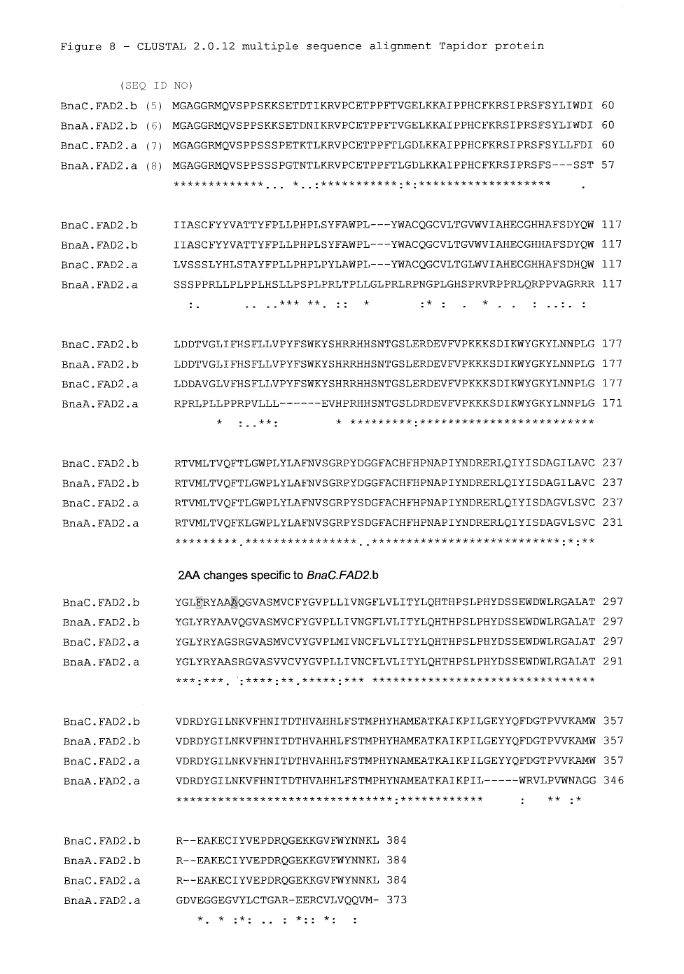

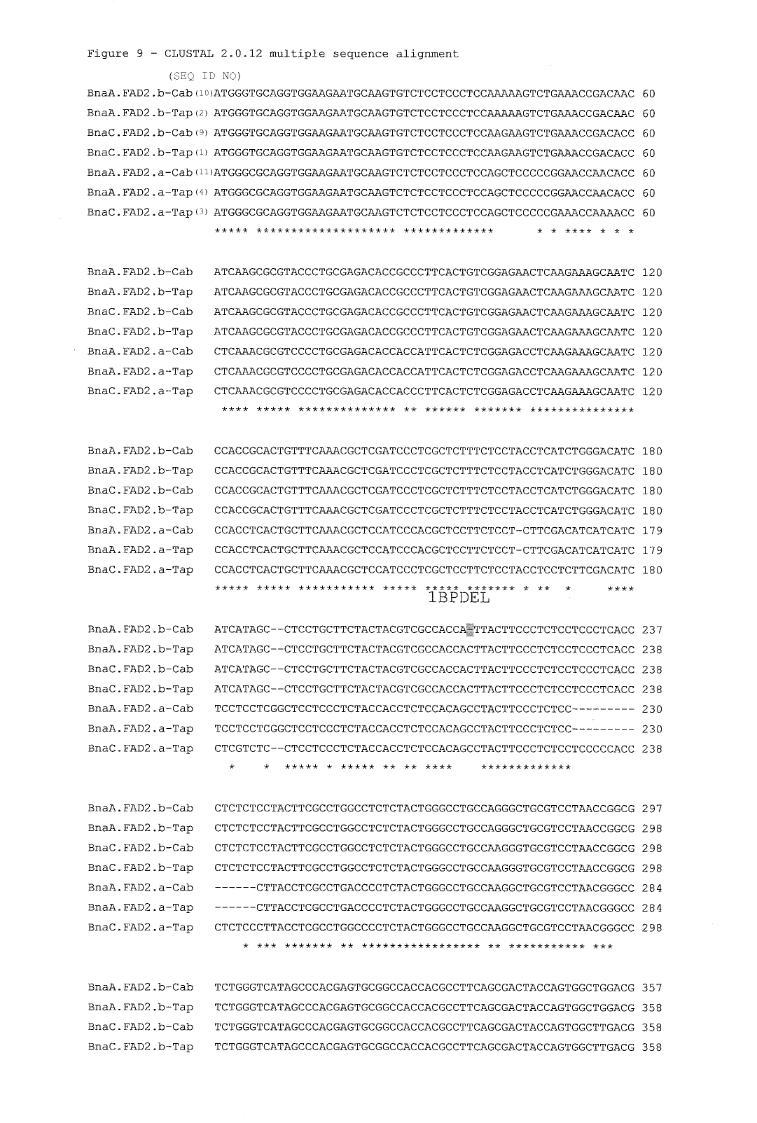

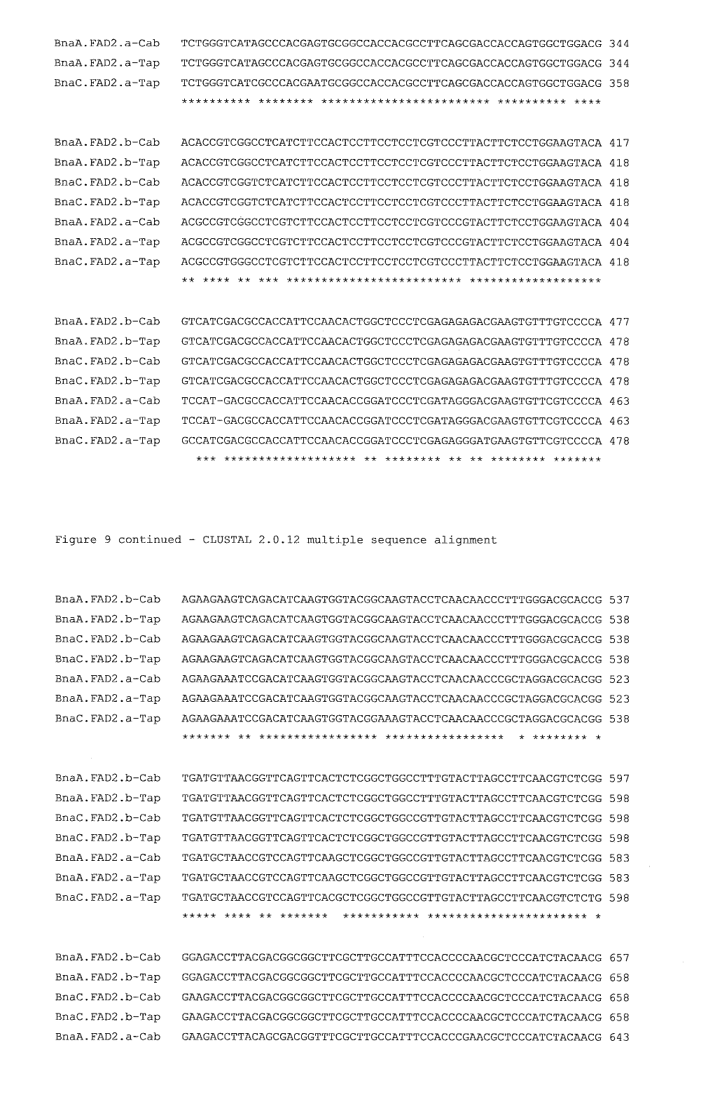

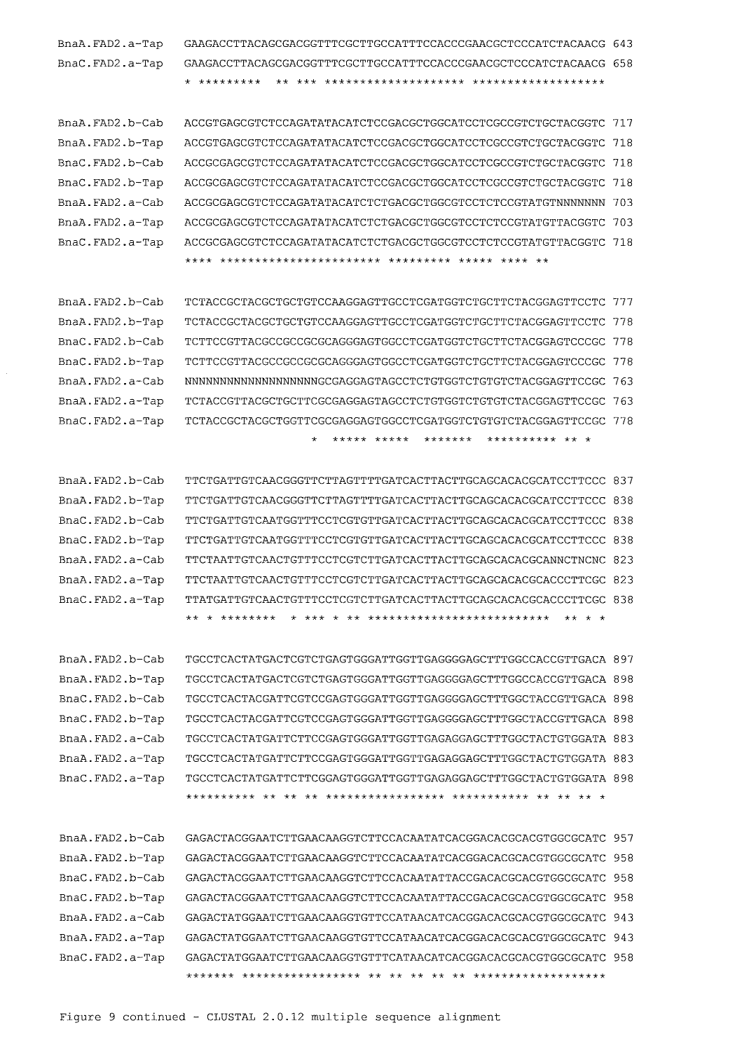

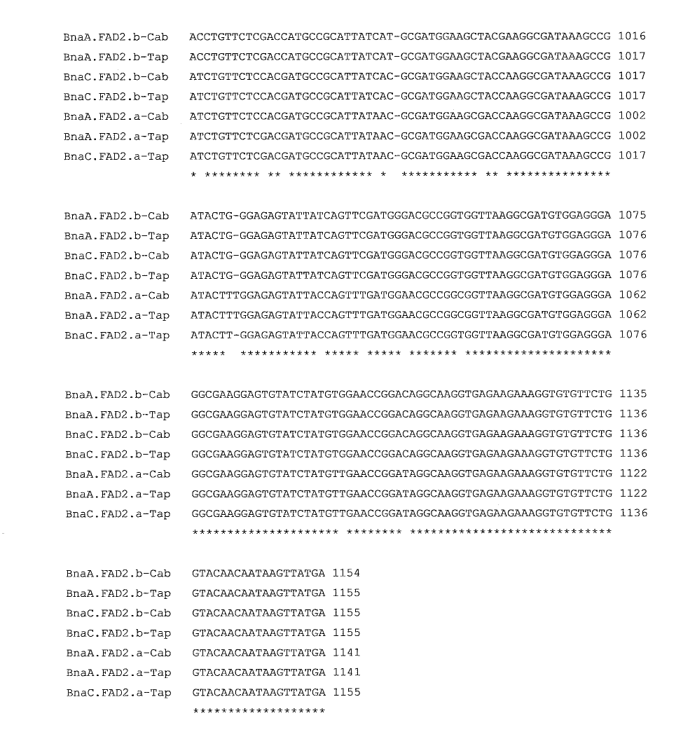

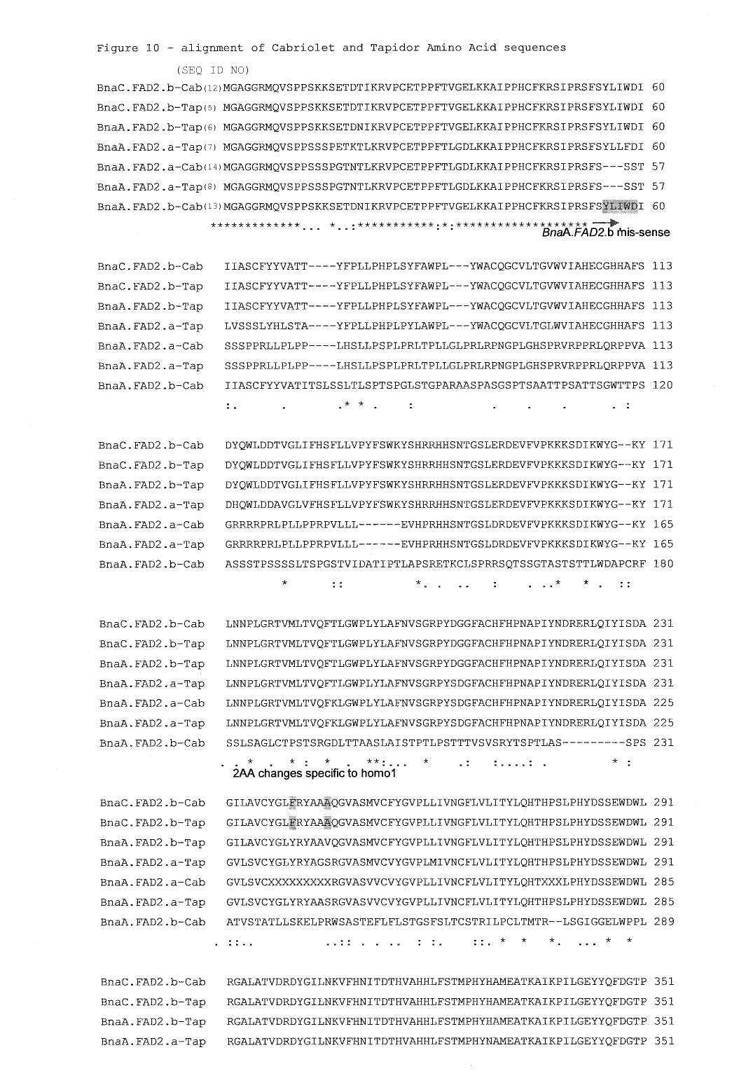

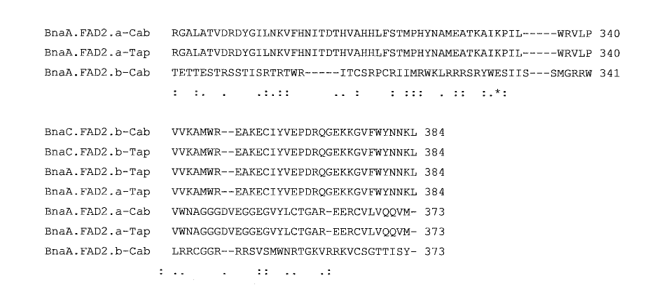

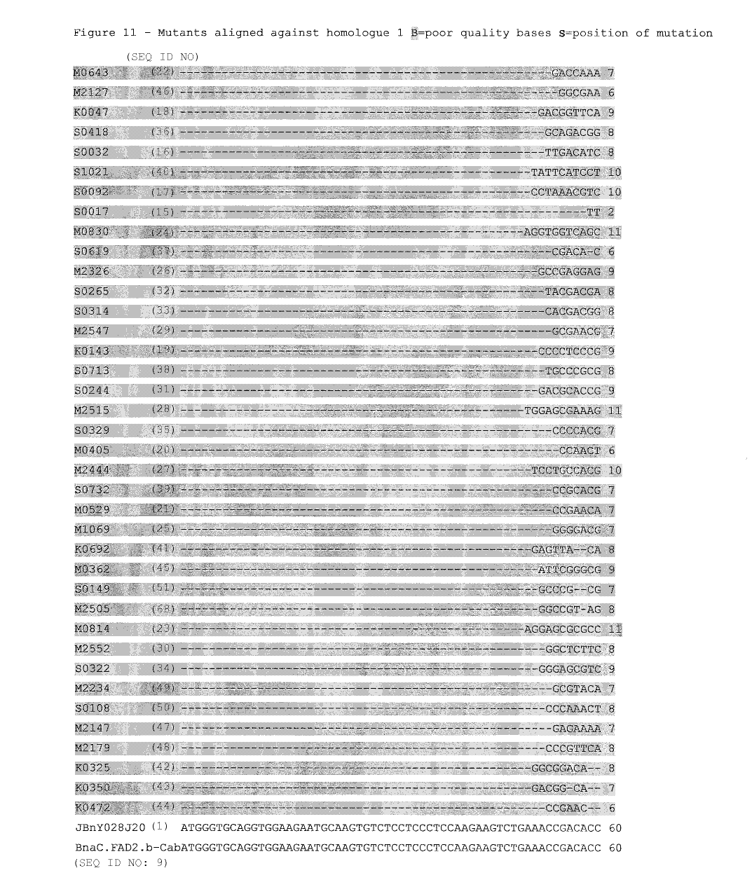

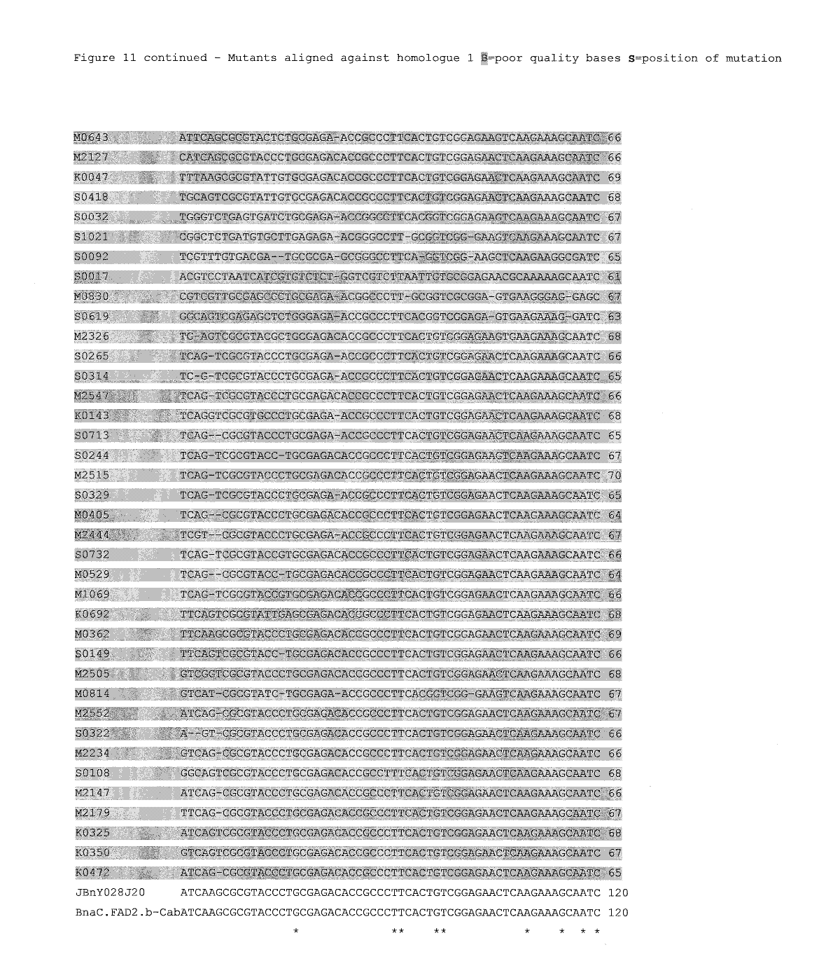

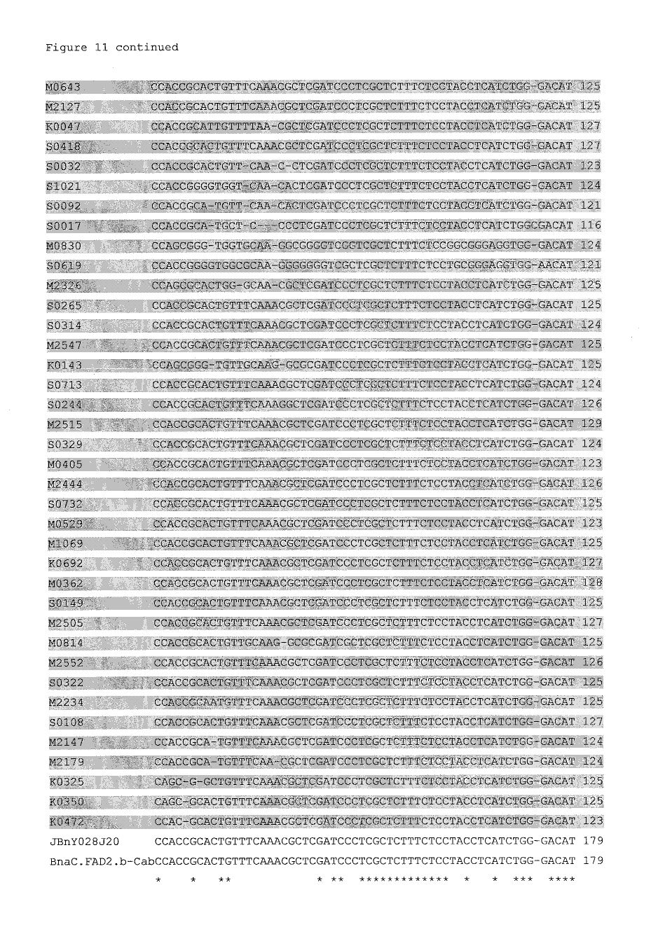

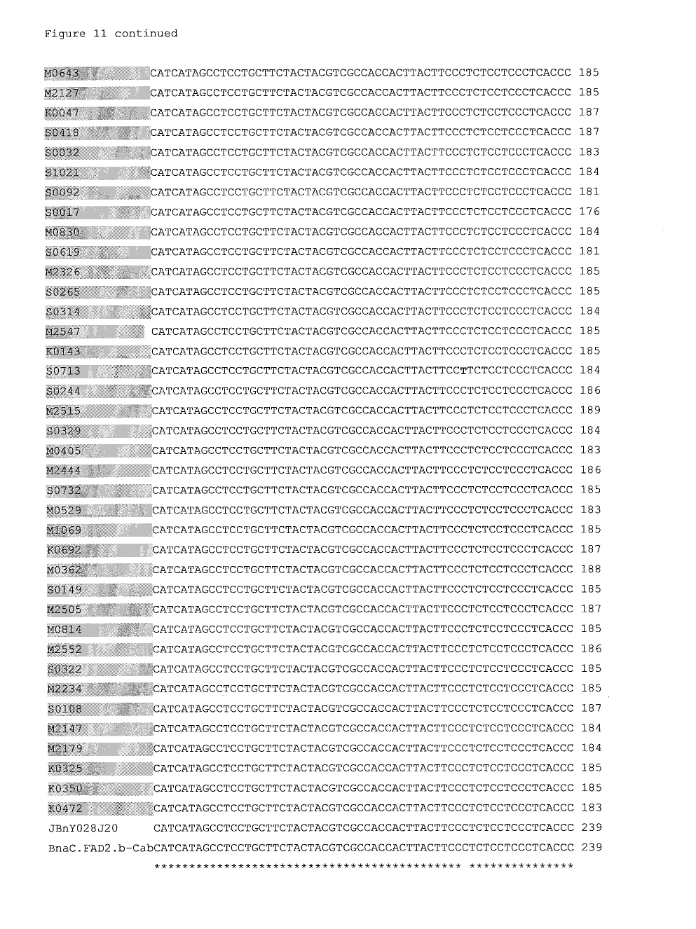

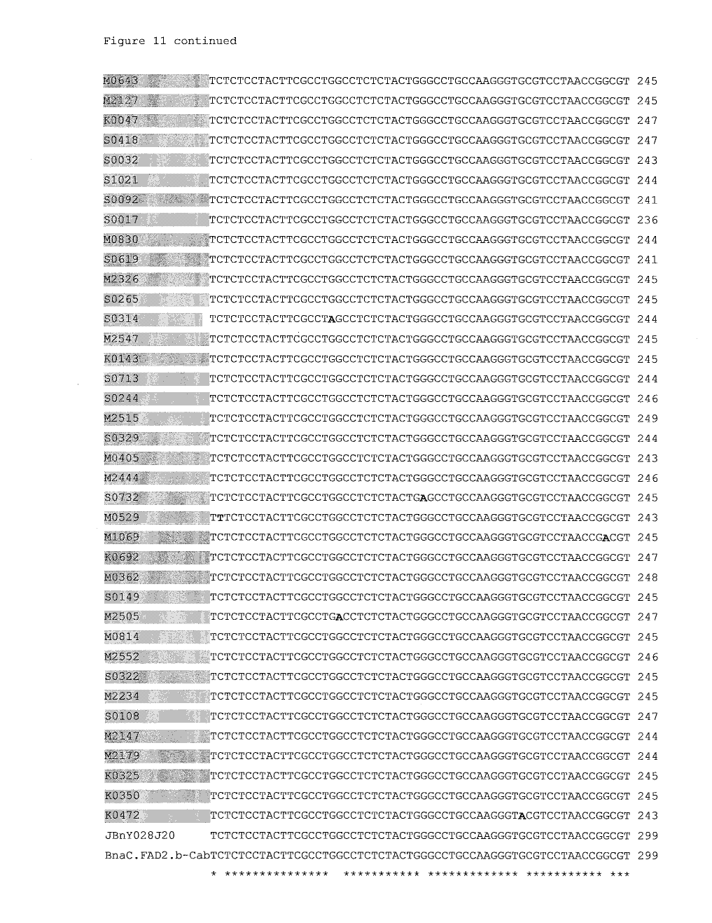

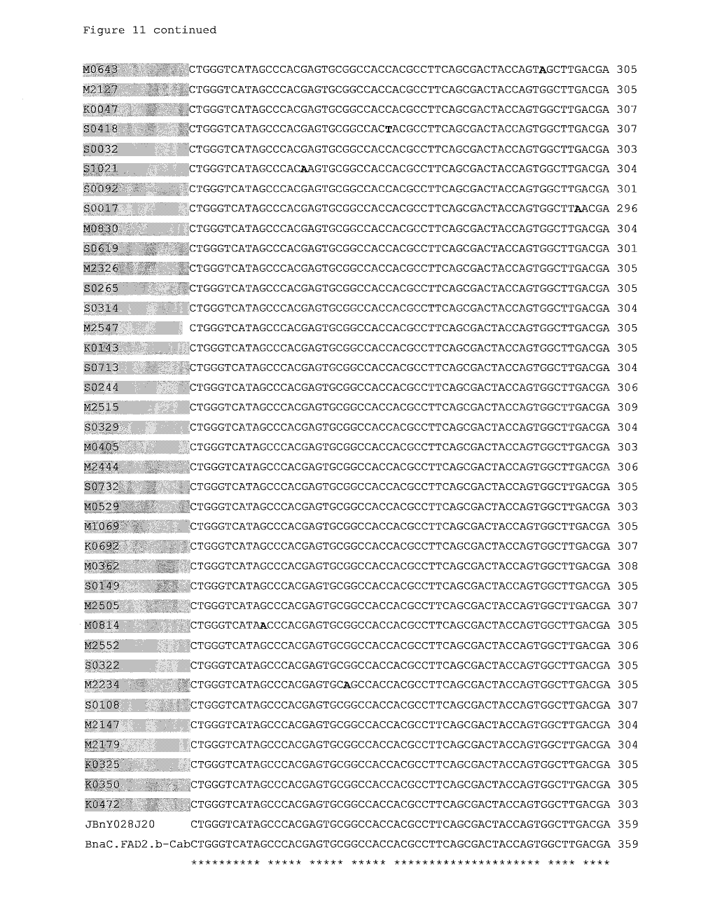

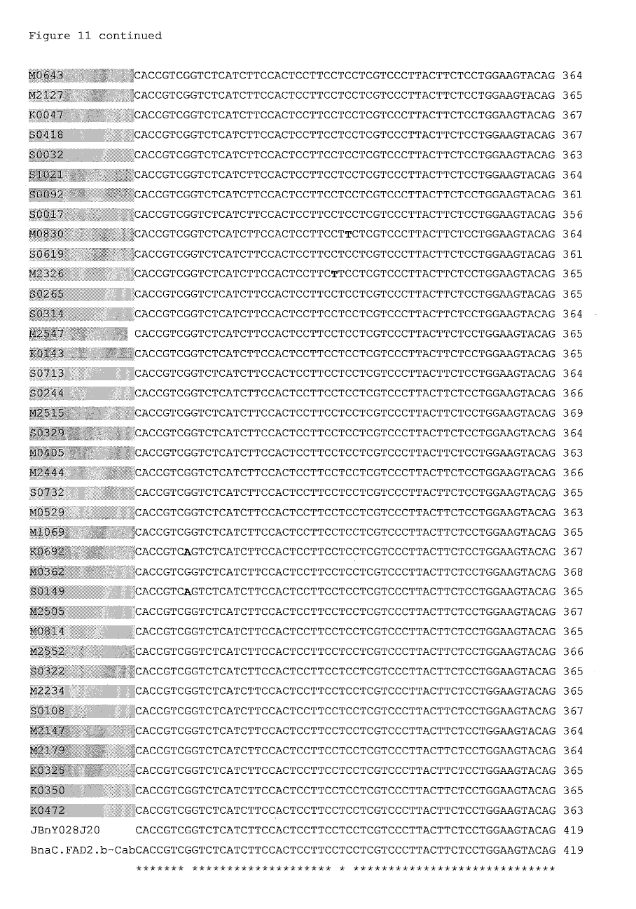

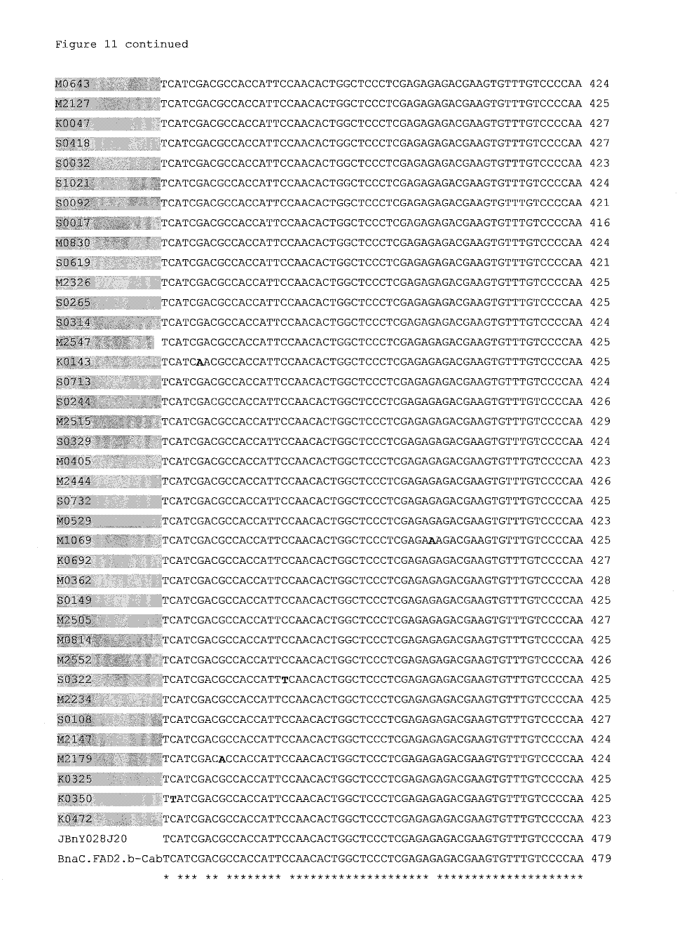

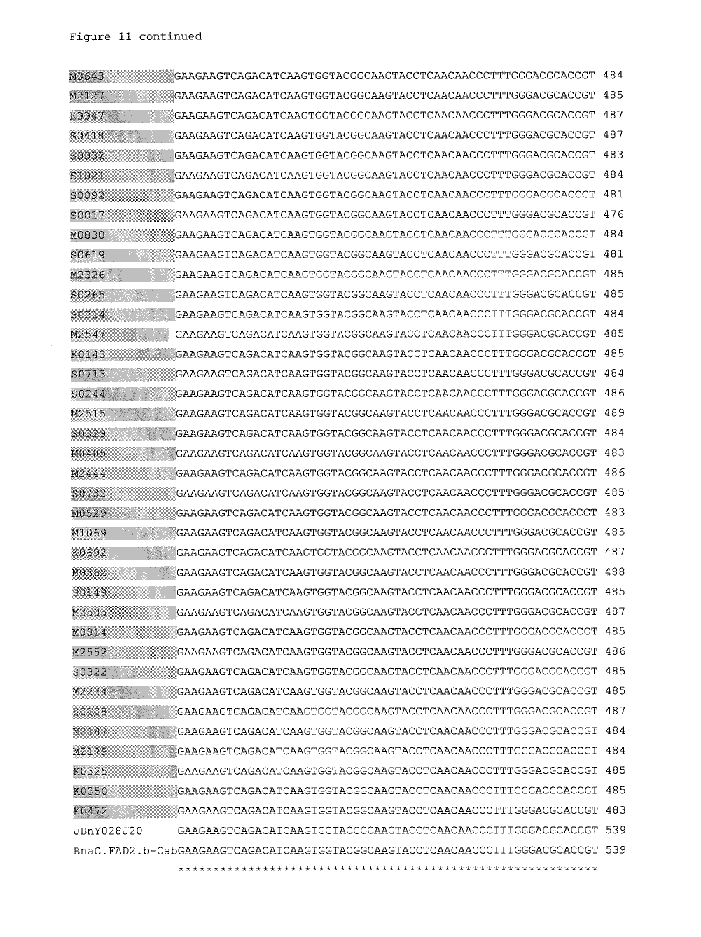

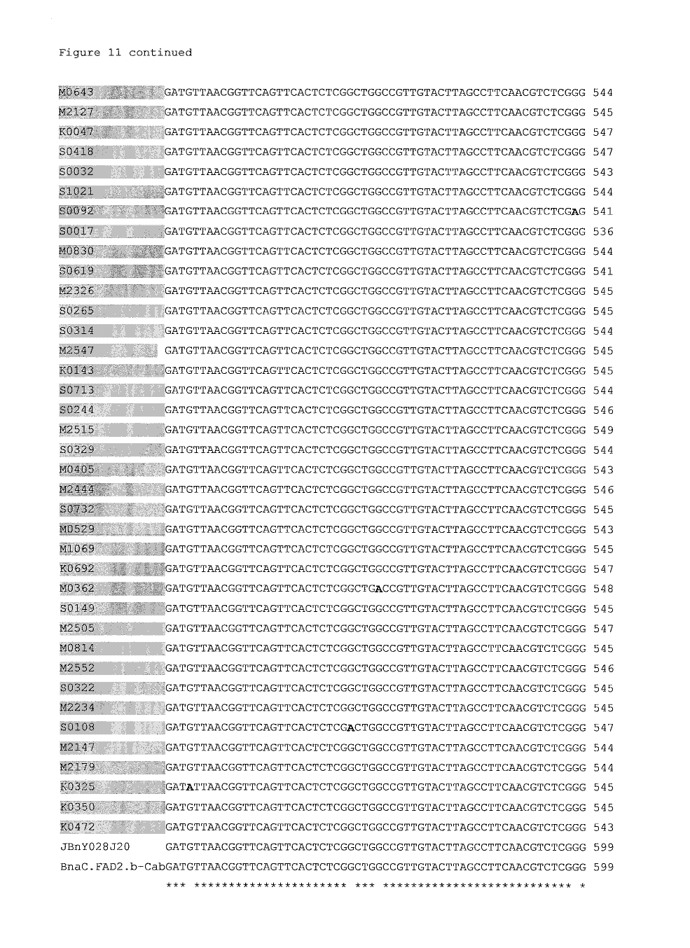

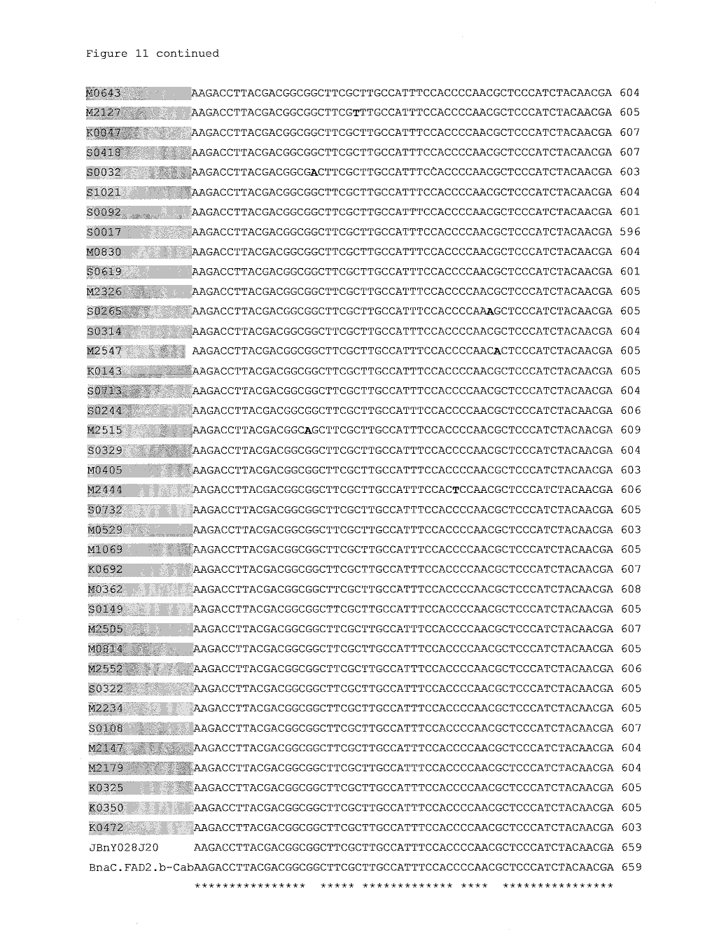

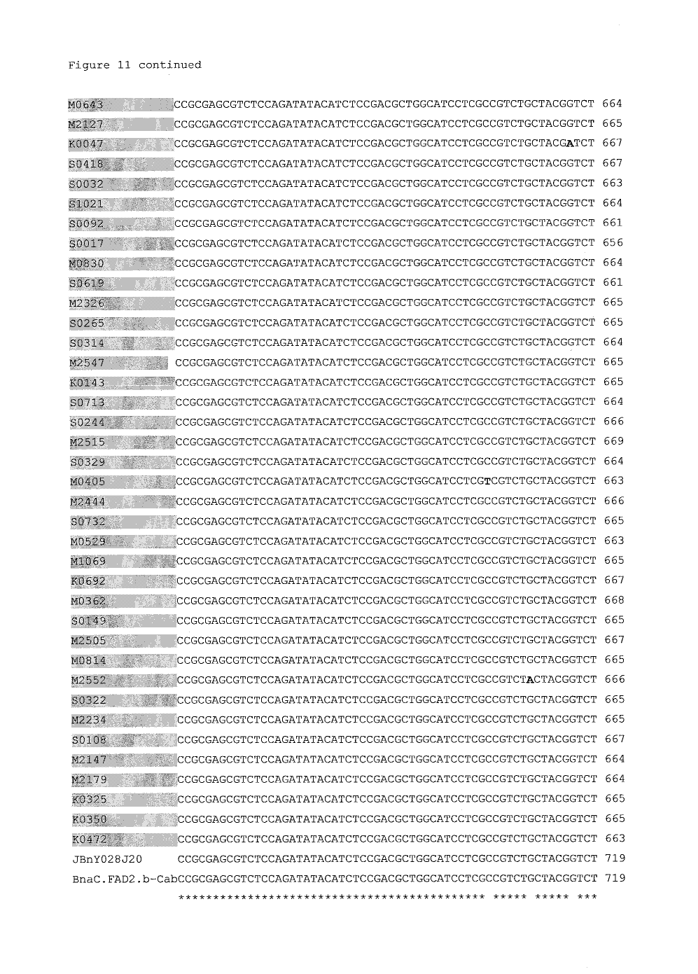

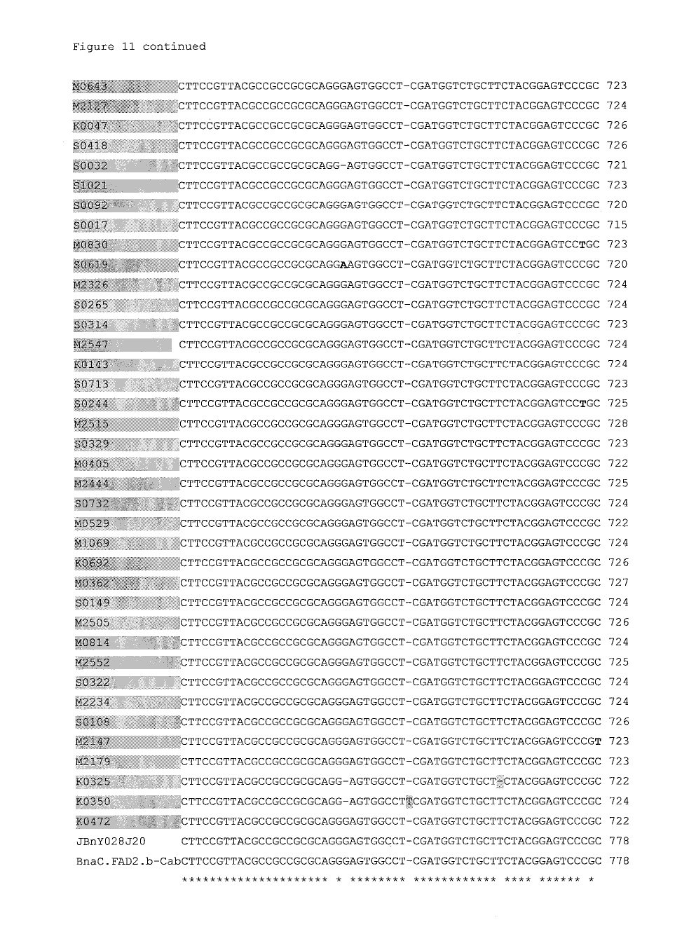

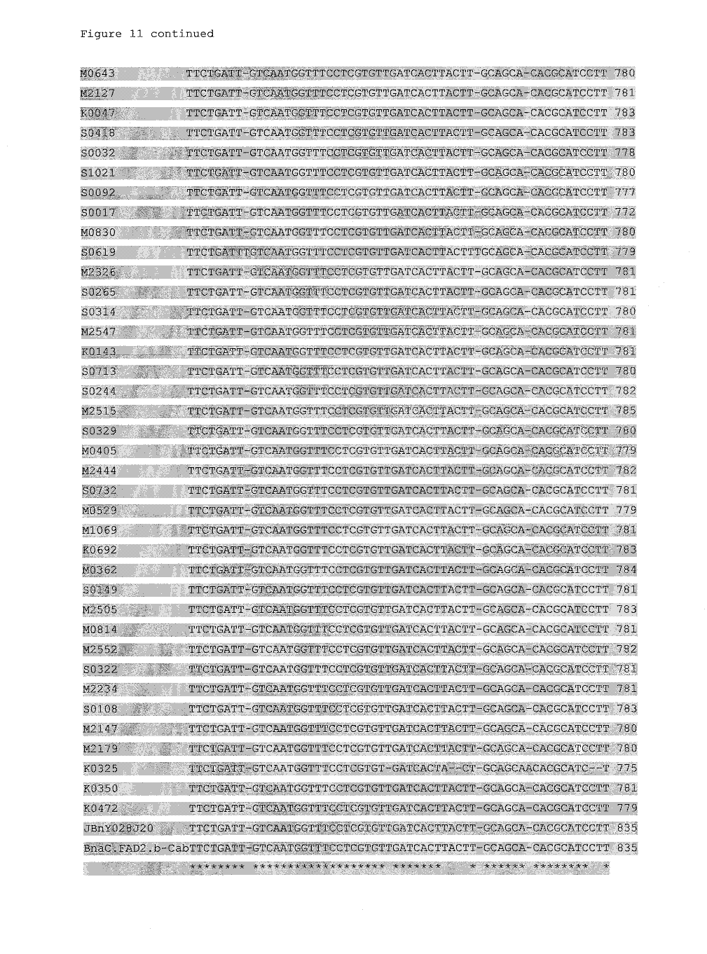

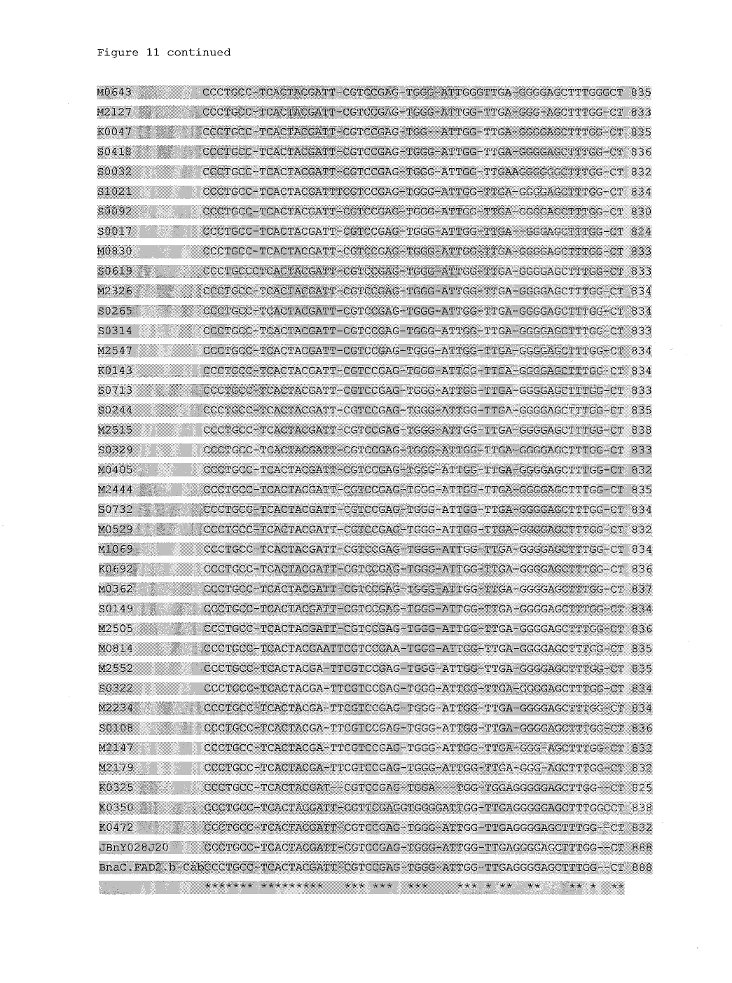

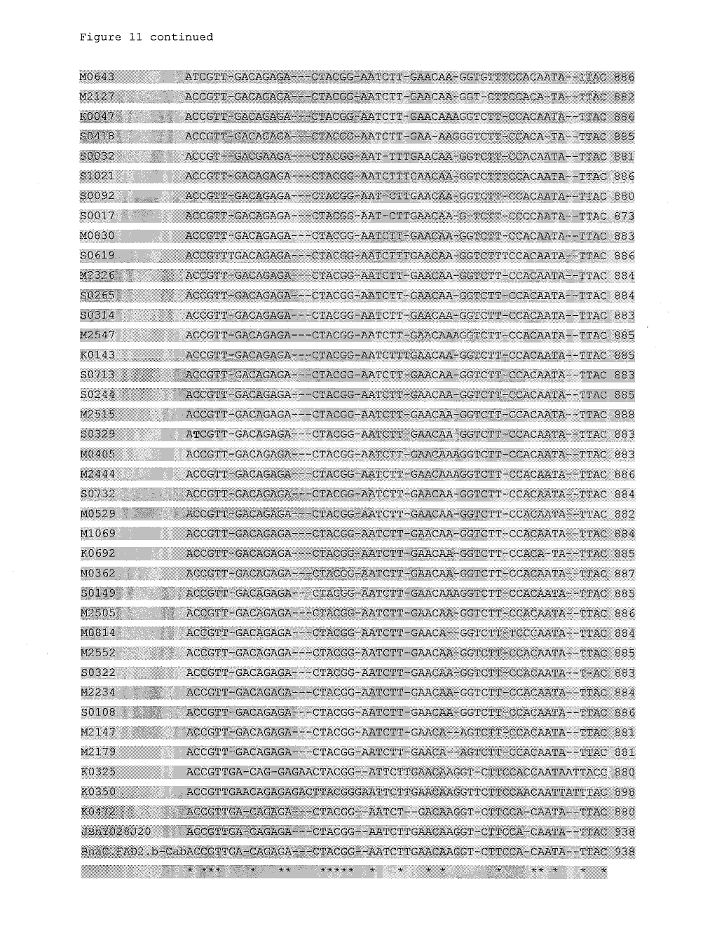

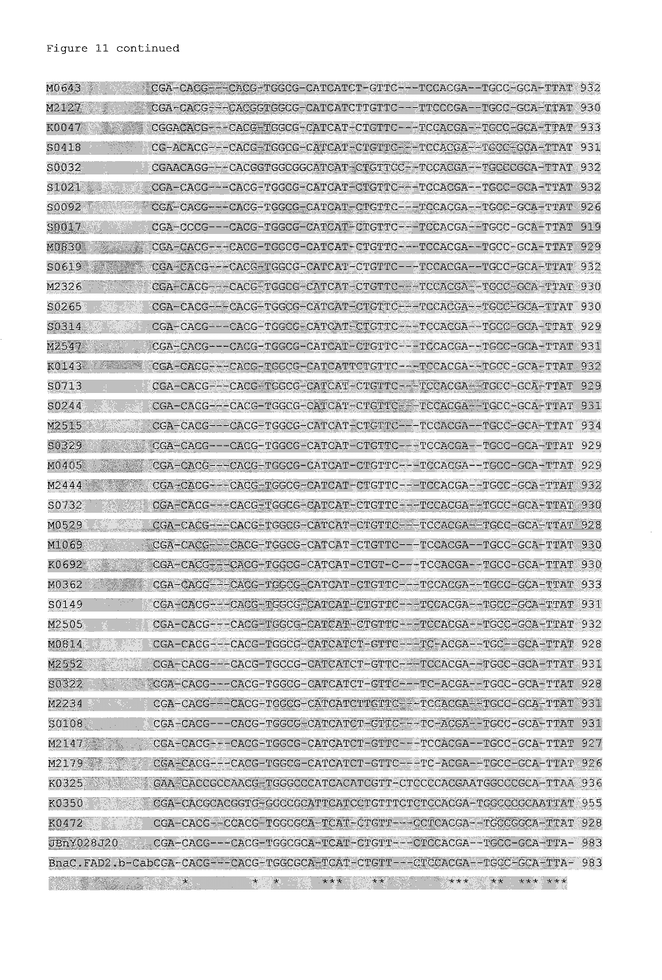

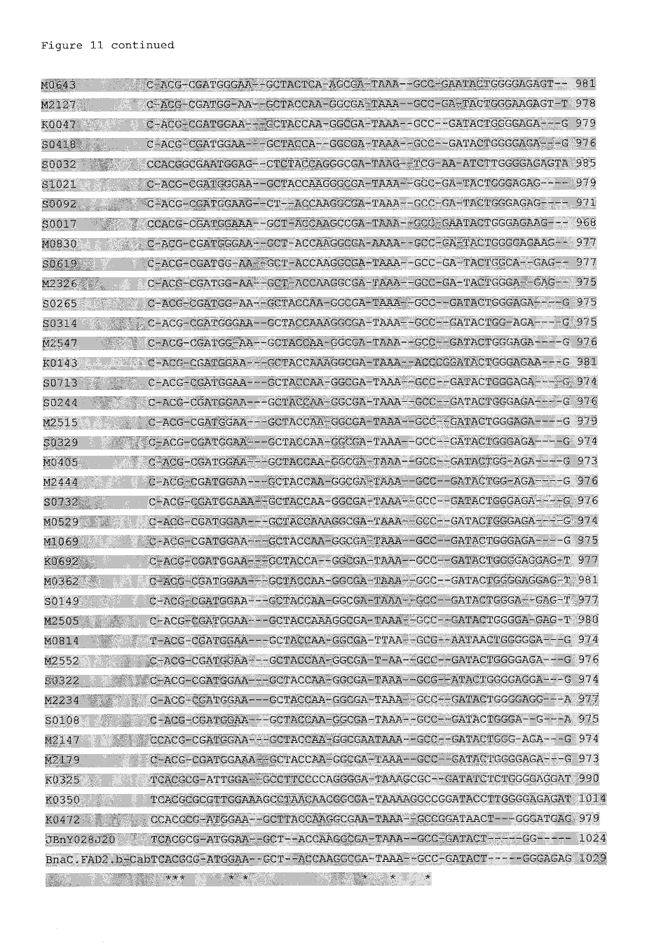

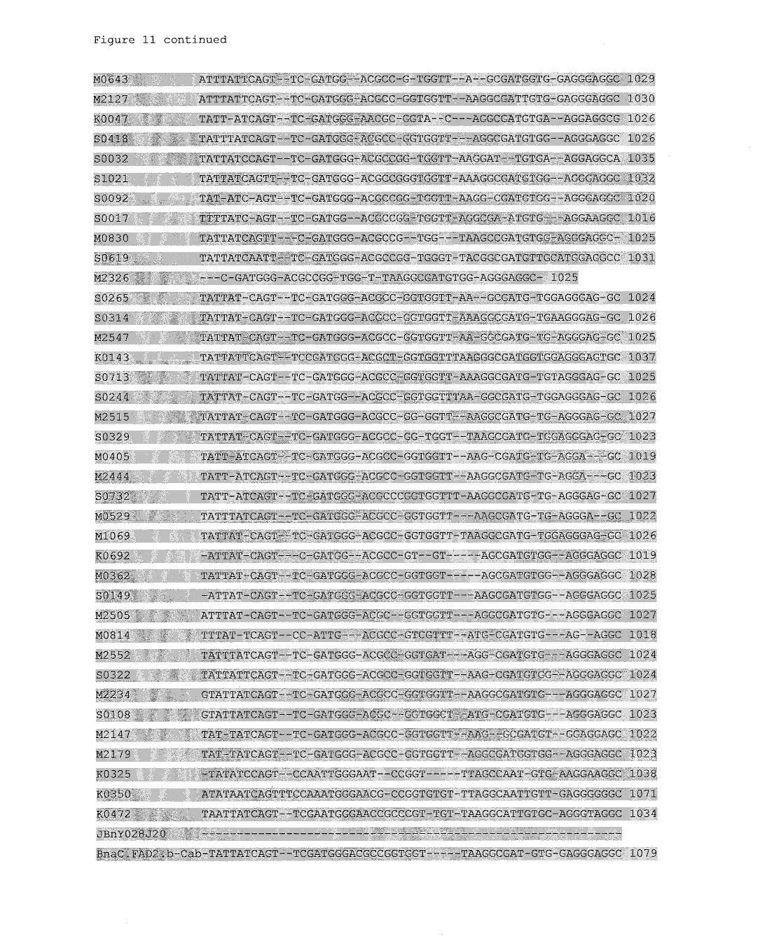

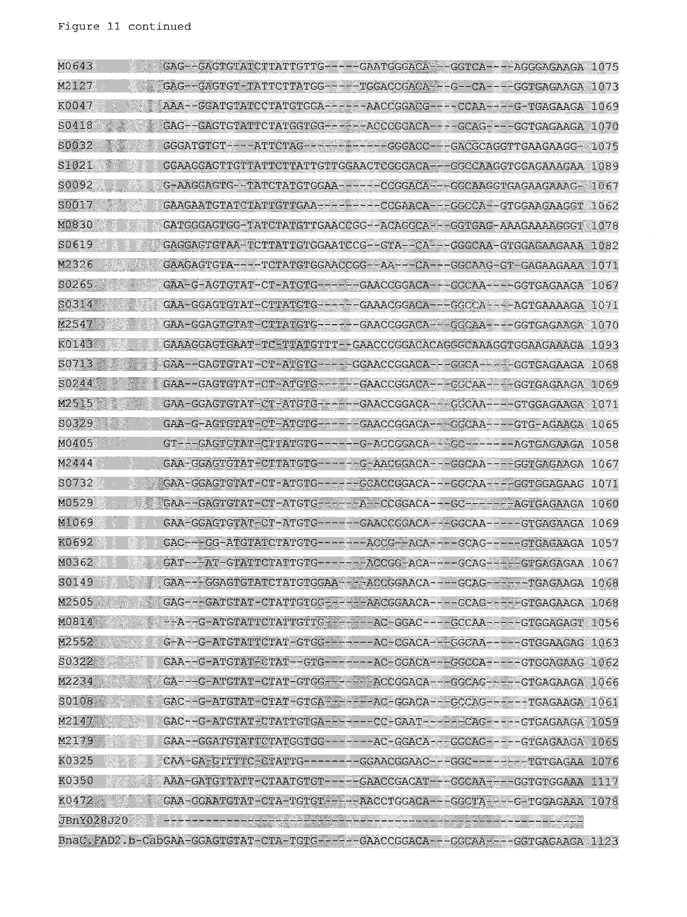

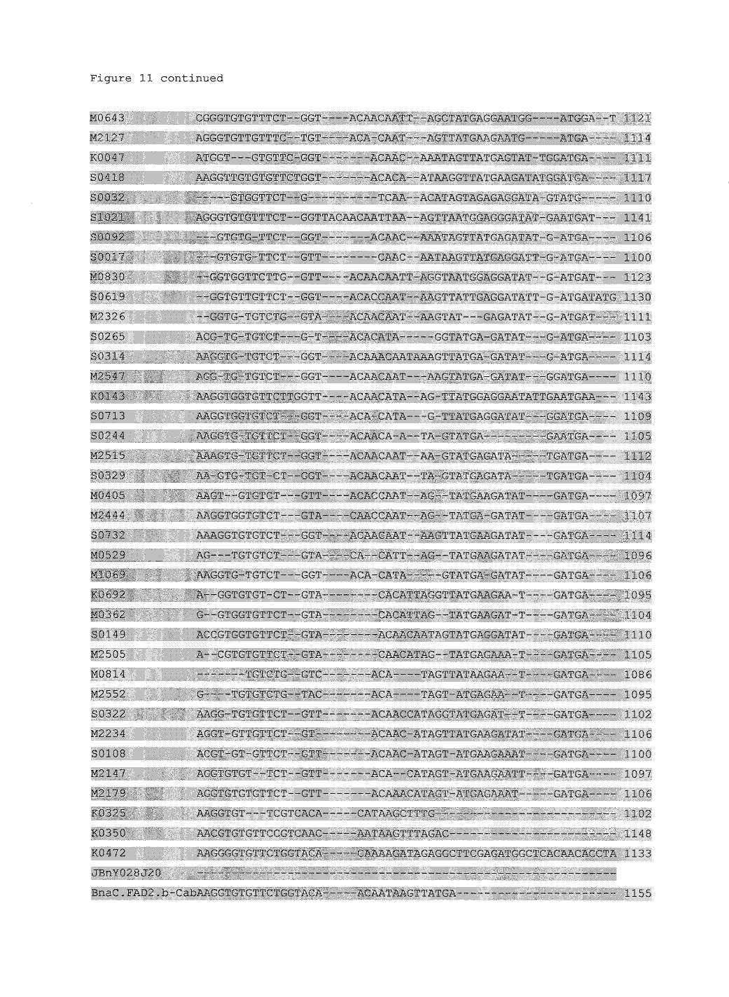

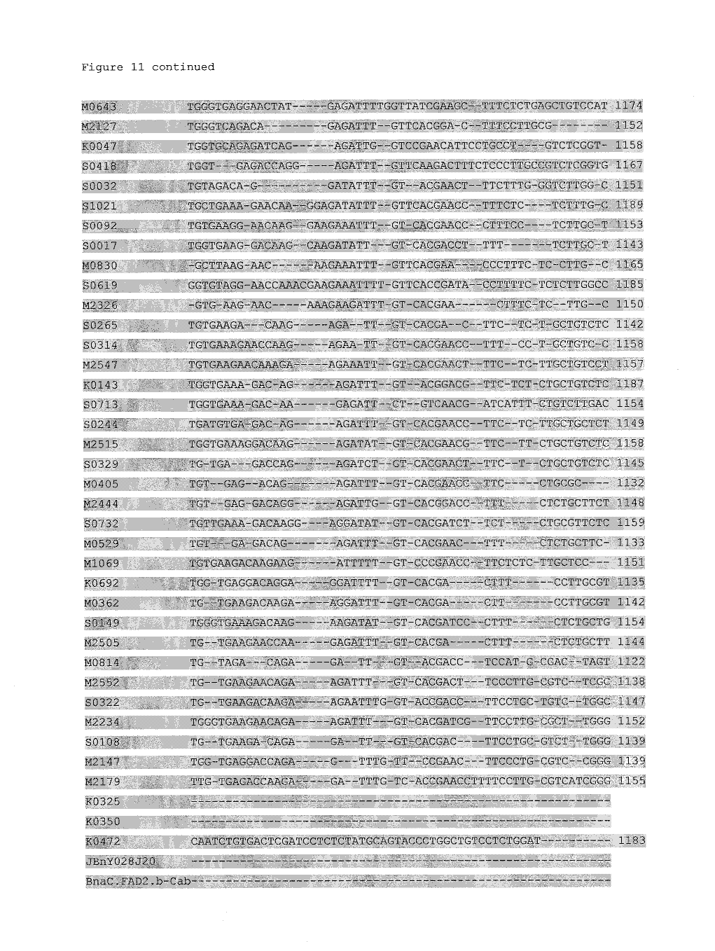

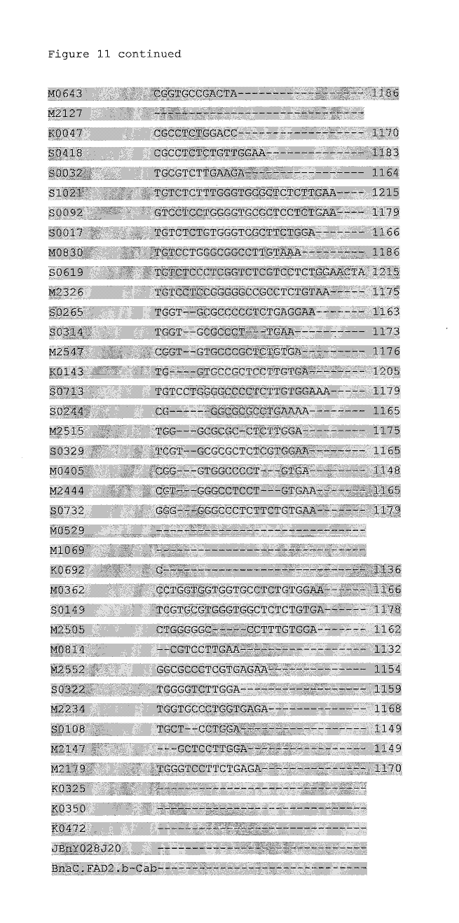

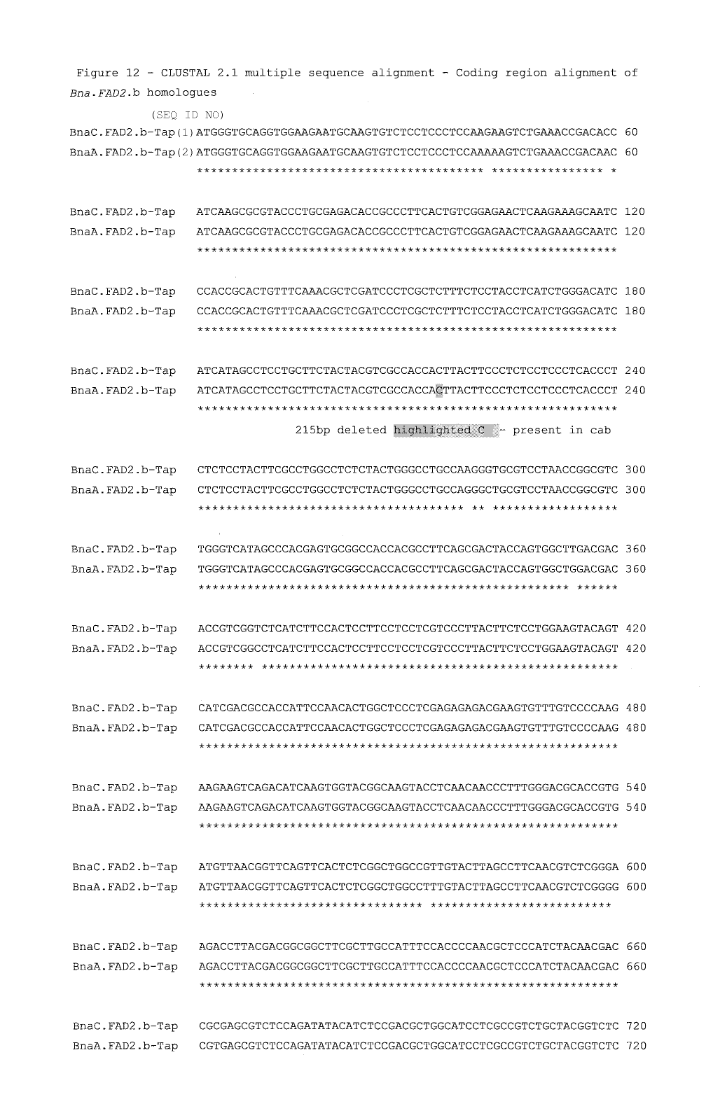

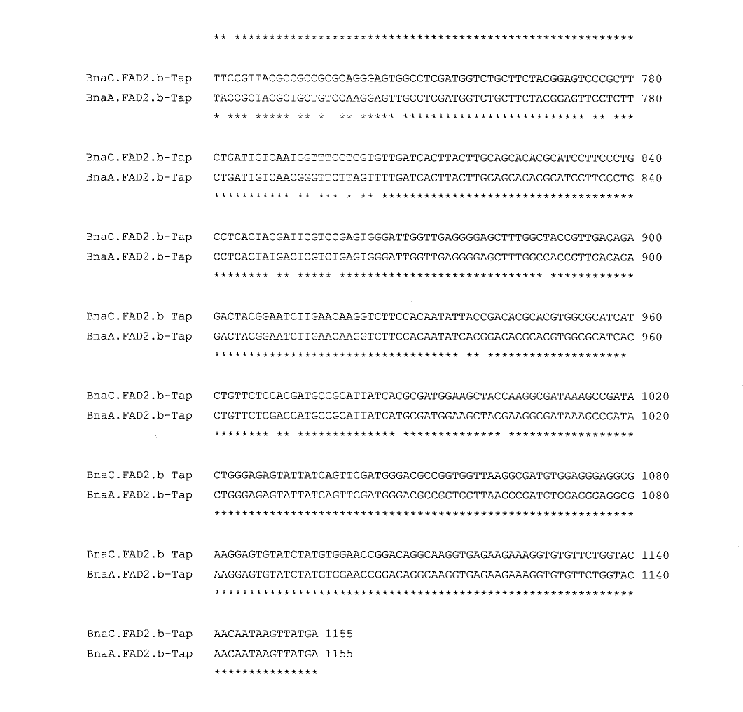

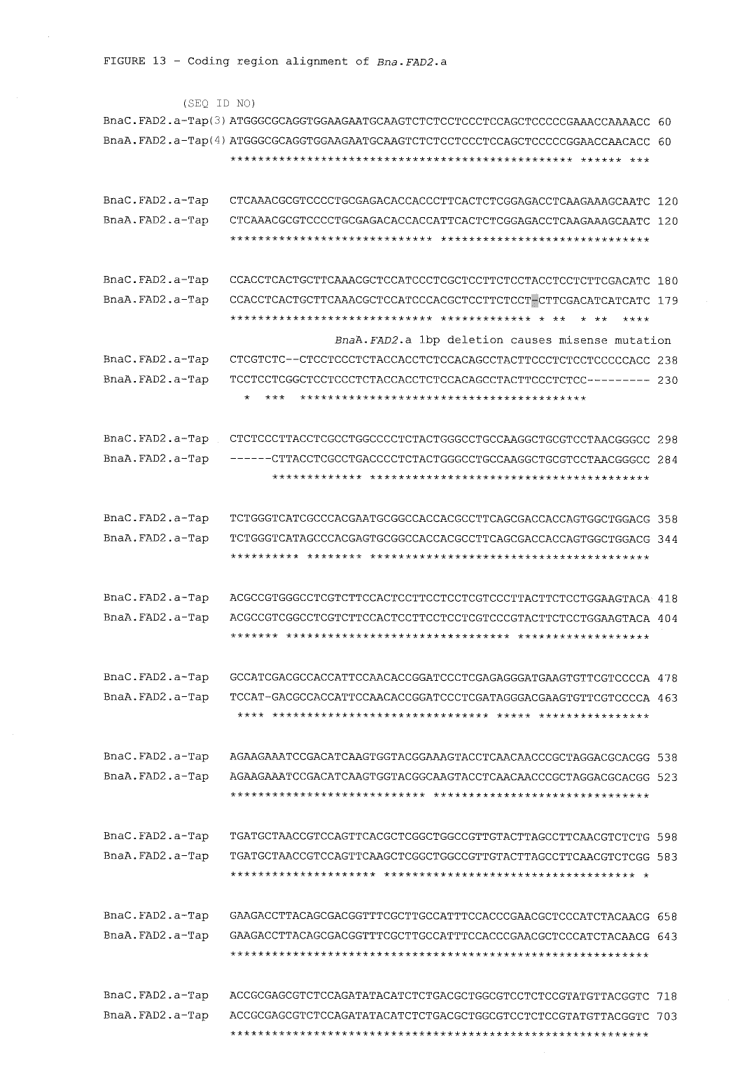

In the disclosure which follows, we demonstrate that B. napus has in its genome four homologues of FAD2, see SEQ ID. NOs. 1-4 for the nucleotide sequences of the four homologues found in B. napus variety Tapidor, (see FIG. 7 for the nucleotide sequence alignments), with the encoded amino acid sequences provided as SEQ ID. NOs.5-8 (see FIG. 8 for the amino acid sequence alignments). In addition, we demonstrate that B. napus variety Cabriolet has in its genome three homologues of FAD2, i.e. BnaC.FAD2.a has been deleted or replaced with BnaA.FAD2.a sequence, see SEQ ID. NOs. 9-11 for the nucleotide sequences of the three homologues found in B. napus variety Tapidor, (see FIG. 9 for the nucleotide sequence alignments), with the encoded amino acid sequences provided as SEQ ID. NOs. 12-14 (see FIG. 10 for the amino acid sequence alignments). Finally, we provide a wide variety of mutant sequences for the remaining active homologue of FAD2 found in B. napus variety Cabriolet, see SEQ ID. NOs. 15-51, (see FIG. 11 for nucleotide sequence alignments showing the various nucleotide changes found in the new varieties of B. napus produced herein).

Herein, we provide sequences for identifying the various homologues of delta-12 oleate desaturase enzyme, namely, BnaA.FAD2.a, BnaC.FAD2.a, BnaA.FAD2.b, and BnaC.FAD2.b, as well as methods and isolates in which all of the FAD2 genes have been inactivated, mutated, deleted, truncated or otherwise modified, such that there is little or no production of delta-12 desaturase activity and maximal production of oleic acid.

Mutants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed to make the encoded FAD2. Inactivation of a FAD2-encoding nucleic acid sequence or inactivation of a FAD2 enzyme is then verified. This can be done for example by measuring the fatty acid content of a plant or part thereof compared to a control plant which has no mutation. If inactivation of a FAD2-encoding nucleic acid sequence or inactivation of a FAD2 enzyme has occurred, the level of FAD2 activity will be reduced. This can be measured by detecting an increase in 18:1 fatty acid content and/or a decrease in the PUFA (e.g. 18:2 and/or 18:3 fatty acid) content of the plant or part thereof. These methods are only illustrative of the numerous standard techniques known in the art for manipulation of DNA sequences and other known techniques may also be used.

Accordingly, in one embodiment according to the present invention, we disclose herein the increase in 18:1 (oleic acid) content from high oleic variety B. napus “Cabriolet” to at least 80% and decrease in polyunsaturated fatty acid content (PUFA) primarily made up for 18:2 and 18:3 to below 10%. In one embodiment, this is achieved via mutagenisis, for example by EMS (ethyl-methane sulfonate) mediated mutation of the B. napus genome and selection and selfing of viable plants. As noted above, the enzyme principally controlling the desaturation of 18:1 to 18:2 desaturated fatty acid is FAD2, and therefore, abolishing its function reduces and/or eliminates progression down the desaturation pathway.

Existing B. napus Varieties:
a. Phenotype:

We have characterized and compared the fatty acid profile of B. napus “Tapior” (an old European variety) and “Cabriolet” (a high oleic acid variety used in modern oleic acid production). The starting phenotype of these two varieties with respect to fatty acid production is summarized in Table 1 below:

TABLE 1
Mean Percentages of each fatty acid
Tapidor

| | | | | | | | | | | | | | |
|---|---|---|---|---|---|---|---|---|---|---|---|---|---|
| 14:0% | 16:0% | 16:1% | 18:0% | 18:1% | 18:2% | 18:3% | 20:0% | 20:1% | 20:2% | 22:0% | 22:1% | 24:0% | 24:1% |
| 0.08 | 4.97 | 0.32 | 1.85 | 60.4 | 21.7 | 7.71 | 0.66 | 1.08 | 0.06 | 0.34 | 0.02 | 0.20 | 0.13 |

Cabriolet

| | | | | | | | | | | | | | |
|---|---|---|---|---|---|---|---|---|---|---|---|---|---|
| 14:0% | 16:0% | 16:1% | 18:0% | 18:1% | 18:2% | 18:3% | 20:0% | 20:1% | 20:2% | 22:0% | 22:1% | 24:0% | 24:1% |
| 0.07 | 4.37 | 0.35 | 1.42 | 74.5 | 8.91 | 7.21 | 0.56 | 1.38 | 0.05 | 0.31 | 0.03 | 0.21 | 0.14 |

As can be seen from Table 1, variety Cabriolet already shows an increase in 18:1 (74.5%) and decrease in 18:2 (8.2%) due to loss of function of FAD2 when compared to the old cultivar Tapidor (60.4% and 21.7%, respectively). Oleic acid percentages as high as 81-85%+ have been reported in the literature (see Rücker and Röbbelen, 1995 (81%); Wong et al. 1991 (>85%)). Herein, we have selected variants which exhibit improvements over the levels reported in Cabriolet where the improvement is 5% or better in 18:1, and reductions in 18:2 and/or 18:3 content is 5% or better.

b. Genetics:

TABLE 2
BAC clones used for sequencing FAD2 homologues

| | | | Allele name | |
|---|---|---|---|---|
| Homologue | BAC | Linkage group | Tapidor | Cabriolet |
| BnaC.FAD2.b | JBnY028J20 | C5 | -Tap | -Cab |
| BnaA.FAD2.b | JBnB069K15 | A5 | -Tap | -Cab |
| BnaC.FAD2.a | JBnY182P11 | C1 | -Tap | -Cab |
| BnaA.FAD2.a | JBnB090P19 | A1 | -Tap | -Cab |

The above table summarizes the BAC clones we utilized, and the relationship of the FAD2 homologues we identified in two different B. napus germplasms, i.e. that of variety Tapidor and that of variety Cabriolet, see discussion and characterization of these varieties in detail below.

i. Variety Tapidor

We have identified, by sequencing of BACs (see table above), cloned PCR product, allele specific PCR, RT-PCR and mRNA-Seq transcriptome sequencing, from the variety Tapidor, four homologues of FAD2 in B. napus named homologues BnaA.FAD2.a, BnaC.FAD2.a, BnaA.FAD2.b and BnaC.FAD2.b and we have mapped the genes as noted above. We have found that BnaA.FAD2.a, on chromosome A1, is truncated in this variety, and is therefore non-functional. We have found that BnaA.FAD2.b and BnaC.FAD2.a on chromosomes A5 and C1, respectively, encode proteins with high sequence similarity as shown in FIG. 7. These are believed to be functional in variety Tapidor. By contrast, Tapidor BnaC.FAD2.b, chromosome C5, contains two unique amino acid changes which are believed to affect protein function. These are amino acid no's 241 and 246. Accordingly, we have concluded that variety Tapidor contains two fully functional plus one compromised homologue of FAD2.

ii. Variety Cabriolet

We have sequenced via cloned PCR product, allele specific PCR, RT-PCR and mRNA-Seq transcriptome sequencing, from variety Cabriolet, three homologues of FAD2. We have confirmed that BnaA.FAD2.a on chromosome A1, is truncated as in the variety Tapidor. We have confirmed that in variety Cabriolet, BnaC.FAD2.a, on chromosome C1, is absent or is, possibly, replaced by homeologous recombination a second copy of BnaA.FAD2.a. We believe this is the first time this has been reported for variety Cabriolet. We report herein that BnaA.FAD2.b, chromosome A5, contains a 1 bp deletion at coding base position 215, which leads to a loss of protein function from enzyme encoded by this locus. Finally, we have confirmed that BnaC.FAD2.b, chromosome C5, encodes a functional but compromised FAD2 enzyme, as in variety Tapidor.

Accordingly, we concluded, based on the genetic analysis, that in variety Cabriolet, while homologues BnaA.FAD2.a, BnaA.FAD2.b and BnaC.FAD2.a are non-functional, BnaC.FAD2.b remains, encoding a compromised functional delta-12 oleate desaturase, and reduces the oleic acid fraction recoverable from this variety. We therefore set about developing methods for producing and screening B. napus in which BnaC.FAD2.b on chromosome C5 is altered in such a way that the enzyme function is further compromised or lost.

We characterized, by sequencing, wild type FAD2 sequences and mutated sequences, produced and characterized as described in detail in the examples which follow. Mutants identified by sequencing of BnaC.FAD2.b PCR product.

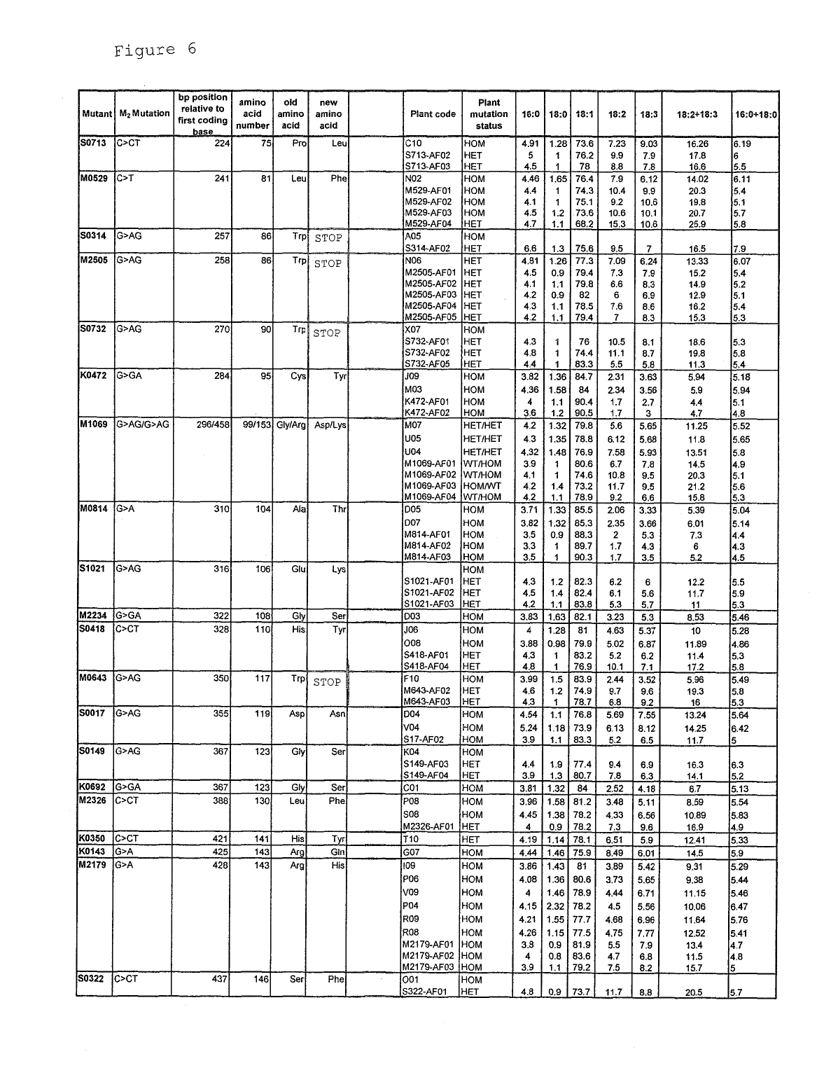

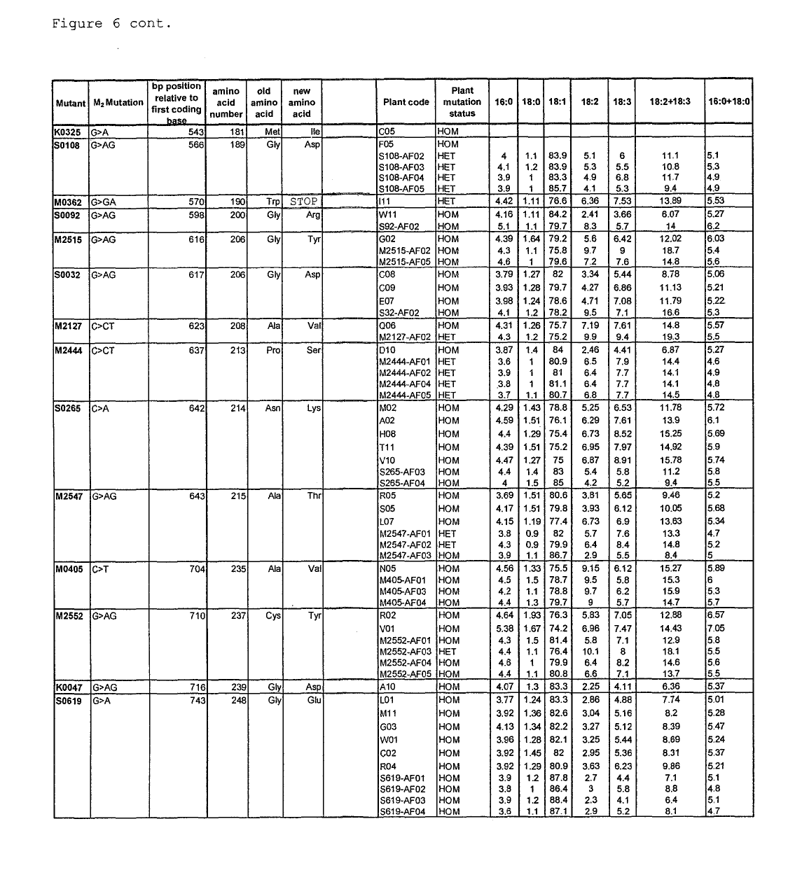

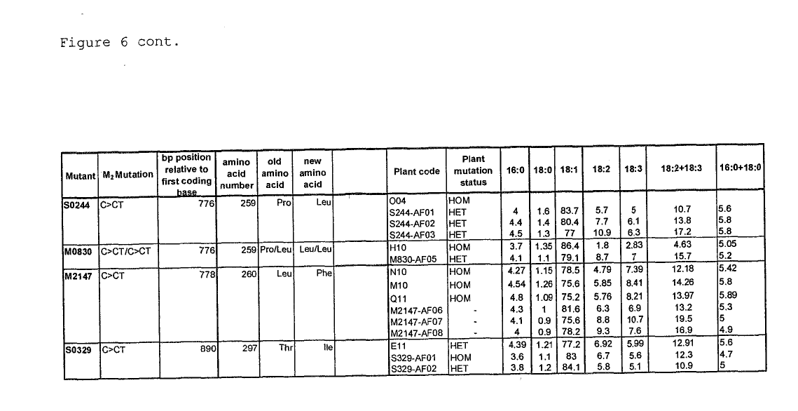

In FIG. 6, we provide a table of mutants identified, showing SNP position/changes, amino acid position/changes and phenotype with respect to fatty acid profile and fraction thereof that is oleic acid and oil profile of given mutants following 1^{st} years trials.

As noted above in the background to this invention, the following patent disclosures provide a wide and diverse series of patent applicants, relating to various efforts to achieve increased oleic acid content in a variety of different plants: U.S. Pat. No. 5,840,946 (Pioneer, “Vegetable Oil Extracted From Rapeseeds Having a Genetically Controlled Unusually High Oleic Acid Content”); WO2004/072259 (Dow Agrosciences, “Altered Fad2 and Fadi Genes in Brassica and the Molecular Marker-Assisted Detection Thereof”); WO2007/107590 (Monsanto, “FAD-2 Mutants and High Oleic Plants”); WO2007/138444 (INRA, “Genetic Markers for High Oleic Acid Content in Plants”); U.S. Pat. No. 7,423,198 and U.S. Pat. No. 7,605,301 (Viterra, “High Oleic Acid Brassica juncea” and “Plant Fad2 Coding Sequence Balancing Fatty Acid Profiling in Edible Oils”, respectively). All of these published patent disclosures are herein incorporated by reference for the purpose of enabling those skilled in the art to utilize in combination the information provided herein with respect to various mutations in Cabriolet BnaC.FAD2.b with mutations or silencing constructs directed to all or the other of the FAD2 homologues.

It should be noted, that there are several homologues of FAD2 (four in oilseed rape) and all must be knocked out in order to get the optimum high oleic, low PUFA phenotype. We have been the first to demonstrate conclusively the presence of the four FAD2 homologues. Mutating any one homologue in oilseed rape would has little or no impact the oil profile; the optimum effect on the oil profile was achieved by us as disclosed herein through the introduction of mutations into or deletion of all four homologues. In that respect, the molecular markers for the three homologues already defective in cultivar Cabriolet are useful, in combination with the sequences provided herein for Cabriolet BnaC.FAD2.b. Breeders utilising the present invention will be interested in the lines disclosed herein with specific mutations that abolish the function of this last homologue of the gene. While Monsanto's Cabriolet high oleic variety only has one functional yet compromised FAD2 of the four genes (as compared to the old European variety Tapidor (which has, apparently, three of four functional FAD2 genes, with one of these being compromised), an important contribution to the art made by the present invention disclosure is that those skilled in the art, based on this disclosure, are able to identify FAD2 mutations in the remaining functional FAD2 gene in Cabriolet.

In addition this disclosure also enables those skilled in the art to localise mutations in all four homologues of the FAD2 genes using the information disclosed herein.

It is further important to note that the variability of oleic acid profile with the different mutations reported herein is related to the severity of the effect of the mutation on the protein itself. Some mutations produce changes that have a greater effect on the proteins' conformation, possibly affecting the enzyme's active site or how it sits in the membrane. Mutations other than a STOP codon can have a very drastic effect. Obviously a STOP mutant should completely abolish function. While report herein the identification of STOP codon insertions into FAD2, BnaC.FAD2.b, only one line was tested as a homozygous STOP M0643, oleic 83.9%, PUFA 5.96%.

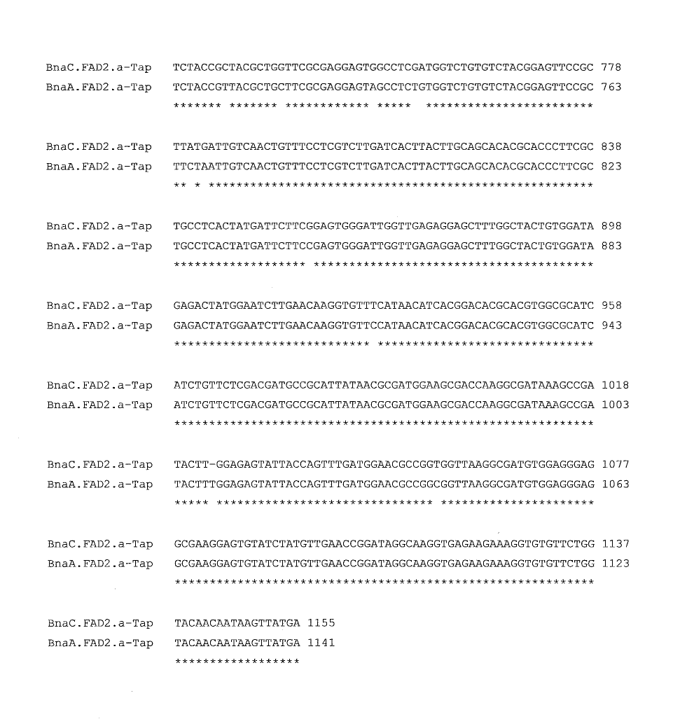

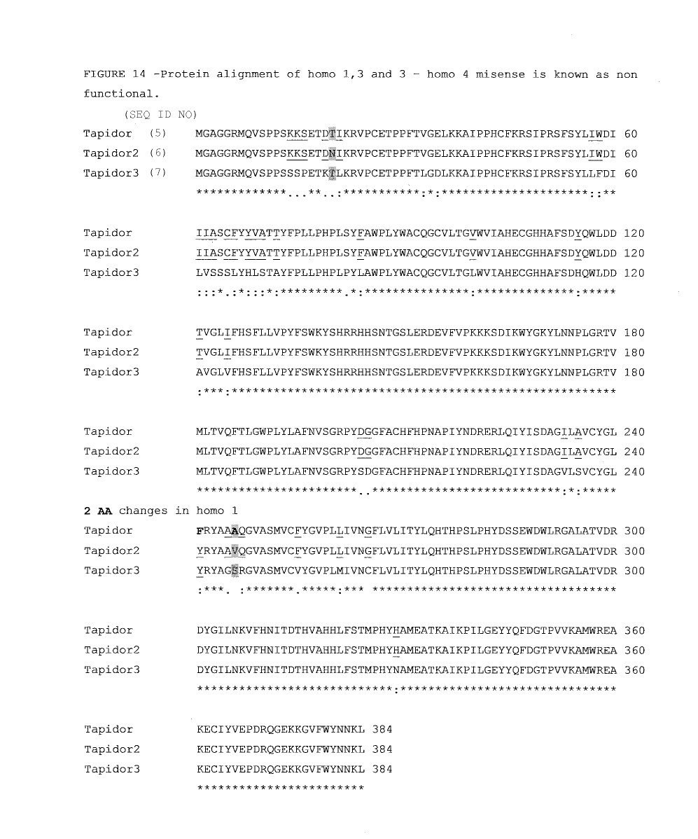

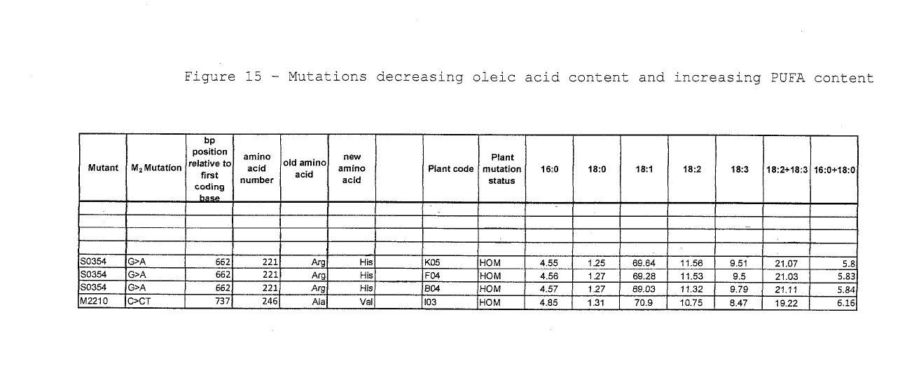

Finally, it is noted that FAD2 appears not to have any additive effect, meaning that there is enough transcript produced in the heterozygous state of one functional gene to produce the amount of protein (enzyme in this case) needed to show the full wild-type phenotype. This is why a significantly reduced low PUFA phenotype, which had been keenly sought for many years, had not been achieved by incremental reduction (i.e. finding in germplasm collections knocked-out alleles of the genes one at a time based on phenotypic screens). Only the combination of three defective genes and the last functional homologue being itself somewhat impaired in enzyme function gave the phenotype of Cabriolet and other low PUFA lines currently in commercial use. Most of the FAD2 BnaC.FAD2.b mutants reported herein have specific changes in amino acids, the most interesting ones abolish enzyme function altogether (probably by affecting the enzyme active site). Interestingly (from the scientific point of view, at least) some mutants have increased enzyme activity, increasing the PUFA content relative to Cabriolet. It is not believed that the phenotypic impacts of these amino acid changes could have been predicted. Thus, it would be wrong to say that one could predict the effect of any given mutation, as some of them can even increase the PUFA fraction, while others reduce the PUFAs and increase the Oleic acid fraction. FIG. 15 shows a selection of mutants from the population where amino acid changes increase the PUFA fraction produced. The change described in mutant M2210 reverts one of the amino acids believed to reduce FAD2 function in BnaC.FAD2.b to the amino acid observed in BnaA.FAD2.b and results in reduced oleic acid content from increased FAD2 functionality in this mutant.

### EXAMPLES

While the foregoing disclosure generally describes this invention, the following examples are provided to further describe and enable this invention. It will be appreciated, however, that these examples and the specifics provided therein are non-limiting and those skilled in the art could vary or use equivalent methods, apparatuses and systems, without departing from the heart of the invention.

### Example 1

### Identification and Characterization of FAD2 Homologues in Brassica Napus

### i) Selection of BACs as Templates for the Amplification of Gene-Specific Probes

Basic local alignment search tool (BLAST) alignment of end sequence data from Arabidopsis BAC clones, developed in the Brassica Investigating Gene Function (IGF) project (http://brassica.bbsrc.ac.uk/IGF/), have been used to calculate the ‘likely’ alignment of the BAC on the Arabidopsis pseudo-chromosome. Positions have also been inferred from the Munich Information Centre for Protein Sequences (MIPS) annotation of the pseudo-chromosome. Using this data it is possible to calculate which gene models the BAC clones are expected to contain.

This information, available at http://brassica.bbsrc.ac.uk/IGF/?page=body/query_clone.htm, was used to identify Arabidopsis BACs containing FAD2. Clones predicted to contain the gene of interest were grown up overnight in 5 ml LB (10 g bacto-tryptone, 5 g yeast extract, 10 g NaCl made up to 1 litre with ddH₂O, pH 7.5) containing 10 mg/ml of kanamycin antibiotic in a 37° C. shaking incubator.

### ii) Small Scale BAC DNA Preparation

Cells were pelleted from 2 ml of overnight BAC culture by spinning for 10 min at 13000 rpm in a micro-centrifuge and pouring off the supernatant. DNA was prepared using the QIAprep Spin miniprep kit (Qiagen Ltd., 2005) following the manufacturers instructions. DNA was eluted into 50 μl of sterile ddH₂O.

### ii) Production of Candidate Gene Probes

Primers SEQ ID. 62: GGGTGCAGGTGGAAGAATG probe f, SEQ ID. 63: TTGTTGTACCAGTACACACC probe r, for probe production were designed from the Arabidopsis FAD2 gene sequence.

To produce the probe, touchdown PCR was carried out in 0.2 ml tubes with a reaction volume of 100 μl containing:

| | | |
|---|---|---|
| 67.6 | μl | ddH₂O |
| 10 | μl | 10 × PCR buffer |
| 3 | μl | MgCl₂ (50 mM) |
| 10 | μl | dNTPs (2 mM) |
| 2 | μl | forward primer (10 mM) |
| 2 | μl | reverse primer (10 mM) |
| 0.4 | μl | Platinum Taq (5 u/μl - Invitrogen) |
| 5 | μl | BAC DNA (⅕ dilution of original DNA |
| | | preparation, ~100 ng) |

PCR was carried out on MWGAG Biotech Primus96 plus thermo-cycler on a touchdown cycle as detailed below with the initial annealing temperature 1° C. higher than greatest primer Tm.

### ‘Touchdown’ Cycle

| | |
|---|---|
| Hot start | 94° C. for 5 mm |
| 15 cycles | |
| Denaturation | 94° C. for 30 s |
| Annealing | 63° C. for 30 s (−1° C./cycle) |
| Extension | 72° C. for 30 s |
| 30 cycles | |
| Denaturation | 94° C. for 30 s |
| Annealing | 53° C. for 30 s |
| Extension | 72° C. for 30 s |
| Final Extension | 72° C. for 7 min |
| End hold temperature | 8° C. forever |

5 μl of the PCR product was checked on 1.5% agarose gel for amplification. The probe was purified to remove unincorporated primer and dNTPs using the QiaQuick PCR purification kit (Qiagen Ltd., 2002) with the DNA eluted into 30 μl of H₂O.

iv) BAC Library Hybridisation with Candidate Gene Probes

Initial screens for BACs containing gene homologues were performed using the B. napus JBnY library developed from the variety ‘Tapidor’. The JBnY library is a TAC library consisting of 73,728 clones with an average insert size of 85 kb constructed using pYLTAC7 as vector. A further screen was performed on a second ‘Tapidor’ library, JBnB. The JBnB library is a large-insert binary vector library consisting of 73,728 clones with an average insert size of 145 kb constructed using pBAC/SACB1 as vector.

Colony hybridisation was performed as described by O'Neill and Bancroft (2000) Comparative physical mapping of segments of the genome of Brassica oleracea var. albogalabra that are homeologous to sequenced regions of chromosome 4 and 5 of Arabidopsis thaliana. Plant J 23:233-243.

Positive BACs were selected for confirmation by Southern hybridization.

### v) 96-Well BAC DNA Preparation

BAC DNA preparation was performed using a method based on Sambrook, et al, 1989 Molecular cloning: a laboratory manual. Cold Spring Harbor Press, Cold Spring Harbor. BACs were picked using sterile toothpicks into Beckman 96 deep-well plates containing 500 μl of 2×YT medium (16 g tryptone, 10 g natural yeast extract, 5 g NaCl made up to 1 litre with ddH₂O) and a selective antibiotic (for JBnB 25 μg/ml chloramphenicol and JBnY 25 μg/ml kanamycin). Plates were incubated overnight at 37° C. in a shaking incubator.

250 μl of the sample was transferred into a Greiner plate and spun in a Qiagen plate centrifuge for 4 min at 2800 rpm to pellet the cells. The supernatant was then discarded by gently inverting the plate onto blue roll.

Cells were re-suspended in 25 μl of re-suspension solution (GTE: 50 mM Glucose, 25 mM Tris-Cl, 10 mM EDTA, pH 8.0) by tapping the plate and left for 5 min. Cells were then lysed by adding 25 μl of lysis solution (0.2 M NaOH, 1% SDS), the samples mixed until clear and again left for 5 min. Twenty five μl of neutralisation solution (3M potassium acetate, pH 5.5) was added and mixed until a glutinous mixture was formed, with the plate then being left to stand for a further 5 min.

The supernatant was spun through a non-sterile Multiscreen GV filter plate at 2800 rpm for 3 min into a clean plate containing 100 μl of propan-2-ol and left to precipitate at room temperature for 20 min. The plate was then spun at 2800 rpm for 20 min to condense the DNA pellets.

The pellets were washed by re-suspending in 110 μl of 70% EtOH, tapping to mix, and again condensed by spinning at 2800 rpm for 10 min. This final wash was repeated and pellets were then dried for 1 h before digestion.

### vi) Fingerprinting Digest and Gel Imaging

Pellets from the BAC DNA preparation were re-dissolved in 15 μl of enzyme mix (5.52 ml ddH₂O, 2 μl Rnase (Rnase One—Promega M4261), 660 μl Buffer B (supplied with Hind III), 220 μl concentrated Hind III (Hind III HC—Roche 1274040)) and digested at 37° C. for 2 h. Two μl of 6×loading dye (15% Ficoll, 0.06% Bromophenol blue, 0.06% Xylene cyanol, 10 mM EDTA) was then added and plates spun uncovered at 2800 rpm for 20 min to reduce the volume to 10 μl.

1.8 μl of digested sample was loaded on to a 121 lane 1% TAE agarose (SeaKem LE, FMC Bioproducts, Rockland, Me., US) gel on the laboratory bench in a wide format system model A3-1 (Owl Scientific, US) gel rig containing 1% TAE. Point eight μl of a mixture of wide range analytical DNA ladder (Promega, catalogue no. DG1931) and molecular weight marker V ladder (Roche, catalogue number 0821705) was loaded every 5^{th} lane starting from the 1^{st} lane. The samples were run at 100V for 10 min before being transferred to the cold room and then run overnight at 55V for 18 h.

Gels were post-stained, shaking for 2 h, in 250 ml of ‘staining fluid’ (20 mM Tris and 0.1 mM EDTA) containing 25 μl of Vistra Green (Amersham Life Sciences, UK). Fingerprints were visualised on the Molecular Dynamics Fluorlmager 595 before being used for Southern Blotting.

vii) Southern Blot and Hybridisation

Southern hybridisation was conducted on positively hybridising clones as described by Rana et al.(2004) (Rana D, Boogaart T, O'Neill C M, Hynes L, Bent E, Macpherson L, Park J Y, Lim Y P, Bancroft I (2004) Conservation of the microstructure of genome segments in Brassica napus and its diploid relatives. Plant J 40:725-733.)

viii) Identification of Homologue Number

Positively hybridising clones were plated onto LB media containing the required selective antibiotic and grown up overnight. Colony PCR was performed using both conserved and degenerate FAD2 primers (SEQ ID. 64: conserved f GAGGGAGGCGAAGGAGTGTATC, SEQ ID. 65: conserved r CAGGAGAAGTAAGGGACGAGG, SEQ ID. 66: degenerate f ATTCCTTCCTNCTNCTNGTNCC, SEQ ID. 67: degenerate r GCTAAGTACAANGGNCANCC) on the touchdown cycle described in iii.

### Colony PCR Reaction:

| | | |
|---|---|---|
| 12.5 | μl | ddH₂O |
| 2 | μl | 10 × PCR buffer |
| 1.3 | μl | dNTPs (2 mM) |
| 2 | μl | forward primer (10 mM) |
| 2 | μl | reverse primer (10 mM) |
| 0.4 | μl | Amplitaq gold (5 u/μl - Invitrogen) |

PCR product was cleaned up sequenced as described below and sequences aligned to determine homologue number.

### PCR Clean Up:

10 μl of sample treated with SAPEXO, 1 μl shrimp alkaline phosphotase (SAP) (Roche—Cat. No. 04898133001) and 0.5 μl exonuclease 1 (EXO) (GE Healthcare—Cat. No. E700732). Samples incubated at 37° C. for 30 min before denaturing at 80° C. for 10 min. Samples were then submitted to the sequencing provider.

### Sequencing Reaction:

Ten microlitre sequencing reactions performed in 0.2 ml tubes using an adjusted protocol from the BigDye v3.1 terminator cycle sequencing kit (Applied Biosystems, 2002).

### Reactions Contained:

| | | |
|---|---|---|
| 1 | μl | PCR product (2-3 ng/μl per 100 bp) |
| 1 | μl | BigDye v3.1 |
| 1.5 | μl | 5 × sequencing reaction buffer |
| 1 | μl | primer (2 μM) |
| 5.5 | μl | ddH₂O |

### Sequencing Cycle:

| | |
|---|---|
| Denaturation | 96° C. for 1 min |

Cycles × 25

| | |
|---|---|
| Denaturation | 96° C. for 10 s |
| Annealing | 50° C. for 5 s |
| Extension | 40° C. for 4 min |
| Cool | 4° C. for 10 min |
| End-hold temperature | 10° C. forever |

One BAC from each identified homologue group was fully sequencing by a service provider.

Confirmation of Homologue Number in B. napus

Primers had previously been developed for the generic amplification of FAD2 as described above. A further non-specific primer was developed to extend this product to the stop codon.

Cloning of generic PCR product to confirm all homologues of FAD2 had been identified in the BAC library was performed using the pGEM-T cloning kit (Promega A3600). Sequencing of clones revealed no further homologues.

Expression of Homologues in B. napus

RNA from leaves and developing seeds of the varieties Cabriolet and Tapidor was extracted from developing seed 45 days after flower opening using the Qiagen RNeasy plant minikit (Qiagen 72904). Alternative buffer RLC was used for RNA extraction from seed due to the oil and starch within the starting material. Cloning of generic FAD2 RT-PCR product produced using the Superscript III first strand synthesis system kit (Invitrogen 18080-051) showed all homologues of FAD2 expressed in Tapidor seed. Expression of BnaC.FAD2.a was not observed in Cabriolet seed. Full transcriptome sequencing using single ended Illumina mRNA-Seq system (Illumina RS-100-0801) confirmed these results. Low levels of expression of the homologues seen to be expressed in the seed were observed in leaf tissue samples.

### BnaC.FAD2.a Absence in Cabriolet

To determine the absence of the homologue in Cabriolet PCR was performed for both the upstream and downstream genes. PCR for Tapidor was successful for all primer combinations. Lack of amplification in Cabriolet suggests a region of at least 25 kb around FAD2 where sequence expected from the sequence of clone JBnY182P11 is absent further supporting the absence of this homologue in Cabriolet.

### Mapping of FAD2 Homologues

Development of specific homologue linked markers and mapping of the homologues is described in Smooker A. M., Wells R., Morgan C., Beaudoin F., Cho K., Fraser F., Bancroft I. (2010) The identification and mapping of candidate genes and QTL involved in the fatty acid desaturation pathway in Brassica napus. Theoretical and Applied Genetics http://www.ncbi.nlm.nih.gov/pubmed/21184048. Homologue BnaA.FAD2.a was assigned to linkage group A1 from homology to a mapped fully sequenced B. rapa BAC. Alignment of FAD2 sequences identified the four separate homologues could be divided into two separate homeologue groups (A1, C1 and A5, C5). The 6 bp difference at 41 bp from the start of the coding region as shown in FIG. 7 was utilized to produce selective primers for these two groups of homeologues. Homologue specific primers were then designed around unique SNPs specific to the individual homologues.

Primers used for conducting PCR for the selective amplification of the different FAD2 homologues are shown in Table 3 below.

TABLE 3

| No. | Primer | Sequence | SEQ ID NO | Bp | GC % | Tm |
|---|---|---|---|---|---|---|
| 1 | Sel 1 + 2 f | GTCTCCTCCCTCCAAAAAGT | 52 | 20 | 50.0 | 54.9 |
| 2 | Sel 1 + 2 new | GTGTCTCCTCCCTCCAAA | 53 | 18 | 55.6 | 51.9 |
| 3 | FAD2 3 + 4 start | CTACAGAAACAAACATGGGC | 54 | 20 | 45.0 | 53.1 |
| 4 | Sel 3 + 4 new | GTCTCTCCTCCCTCCAGC | 55 | 18 | 66.7 | 53.4 |
| 5 | Sel 3 + 4 f | CTCTCCTCCCTCCAGCTCCC | 56 | 20 | 70.0 | 62.4 |
| 6 | FAD2 H3 f | CTCTTCGACATCCTCCTCTC | 57 | 20 | 55.0 | 53.3 |
| 7 | Cons f 1698 | CCTCGTCCCTTACTTCTCCTG | 58 | 21 | 57.1 | 58.2 |
| 8 | FAD2 stop | CCTCATAACTTATTGTTGTACCAG | 59 | 24 | 37.5 | 53.9 |
| 9 | FAD2grp13′UTR-r2 | CAAGACGACCAGAGACAGC | 60 | 20 | 55.3 | 55.0 |
| 10 | FAD2grp4 3′UTR2 | GAACTCGACAAATTTGCCTG | 61 | 20 | 55.7 | 45.0 |

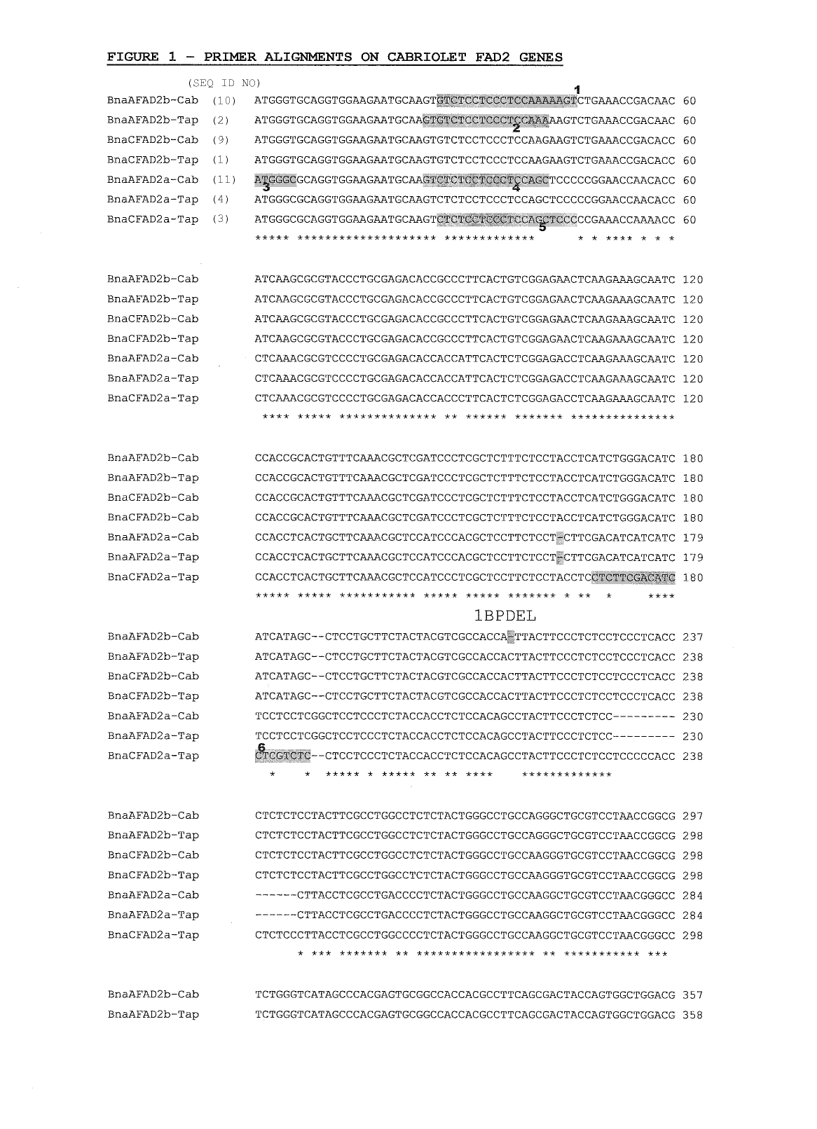

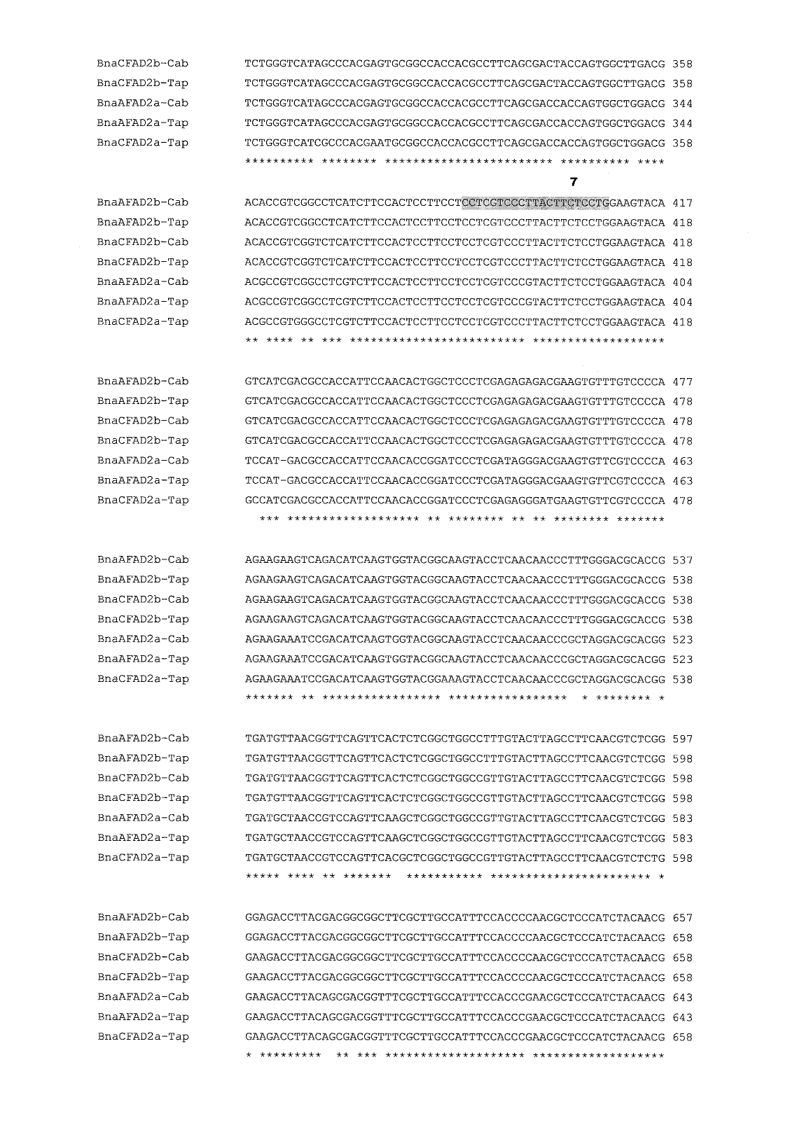

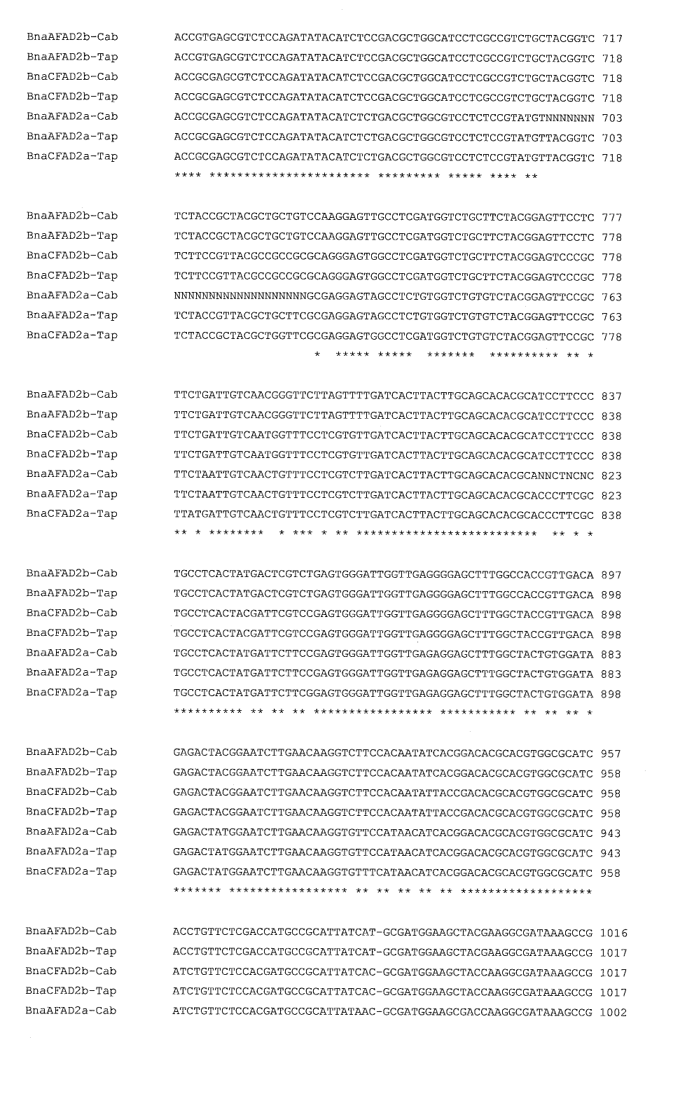

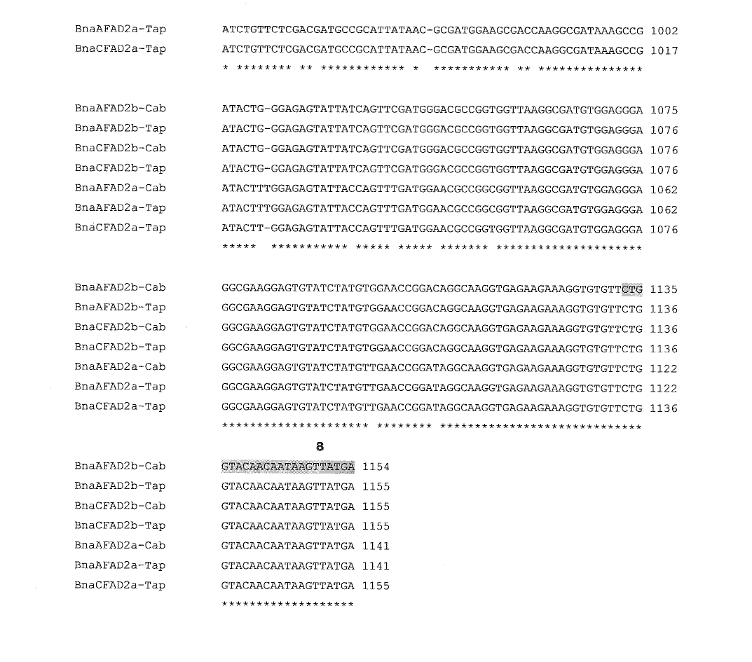

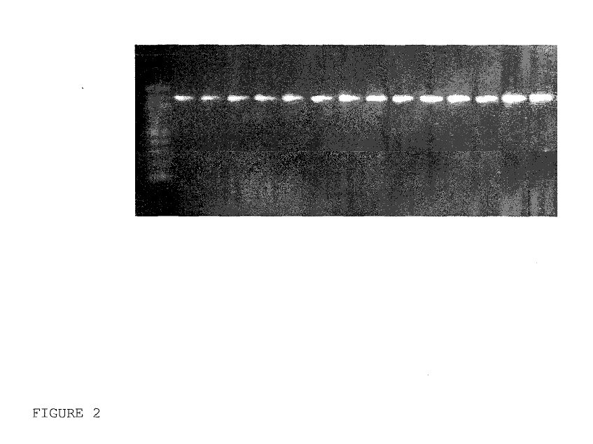

The position of primers on each gene is shown in FIG. 1 (UTR primers not included). Primer combinations to amplify individual homologues are detailed in Table 4 below:

TABLE 4

| Homologue | F primer | R primer | Amplicon size | Notes relative to Cabriolet |
|---|---|---|---|---|
| BnaC.FAD2.b | Sel 1 + 2 f | FAD2grp13′UTR-r2 | 1212 | |
| BnaA.FAD2.b | Sel 1 + 2 new | FAD2 stop | 1133 | |
| BnaC.FAD2.a | FAD2 H3 f | FAD2 stop | 991 | Will not amplify |
| BnaA.FAD2.a | FAD2 3 + 4 start | FAD2 stop | 1173 | May produced mixed Bna.FAD2.a |
| | | | | amplicon in other genotypes |
| BnaA.FAD2.a | Sel 3 + 4 f | FAD2 stop | 1133 | May produced mixed Bna.FAD2.a |
| | | | | amplicon in other genotypes |
| BnaA.FAD2.a | Cons f 1698 | FAD2grp4 3′UTR2 | 966 | |

The PCR protocol was as follows:

### PCR Mix

DNAx (100 ng or 1 μl of qiagen 96 well DNA extraction)

### Primer f (2 μM) 2 μl

### Primer r (2 μM) 2 μl

10×PCR buffer 2 μl
Amplitaq gold (5 u/μl) 0.2 μl
dNTPs (2 mM) 1.3
ddH₂O y

### Total 20 ul

### PCR Cycle

| | |
|---|---|
| Hotstart | 94° C. for 5 min |

Cycles × 35

| | |
|---|---|
| Denature | Ramp 0.5 c/s to 94° C. |
| | Temp 94° C. for 30 s |
| Anneal | Ramp 0.5 c/s to 57° C. |
| | Temp 57° C. for 30 sec |
| Extend | Ramp 0.5 C/s to 72° C. |
| | Temp 72° C. for 1 min |
| Final extend | 72° C. for 10 min |

### Visualisation of Product:

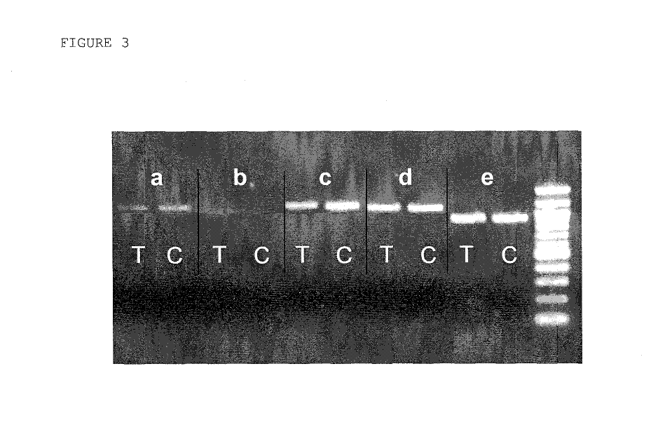

5 μl run on 1.5% agarose gel—see FIG. 3

### Alternative PCR Conditions:

Improved amplification for some amplicons can be achieved by using a 1 KB touchdown PCR protocol however this may lead to a reduction in specificity. Under these conditions BnaC.FAD2.a specific primers will readily amplify BnaA.FAD2.a.

### ‘Touchdown’ Cycle

| | |
|---|---|
| Hot start | 94° C. for 5 rain |

Cycles × 15

| | |
|---|---|
| Denaturation | 94° C. for 30 s |
| Annealing | 63° C. for 30 s (−1° C./cycle) |
| Extension | 72° C. for 1 min |

Cycles × 30

| | |
|---|---|
| Denaturation | 94° C. for 30 s |
| Annealing | 53° C. for 30 s |
| Extension | 72° C. for 1 min |
| Final Extension | 72° C. for 7 min |

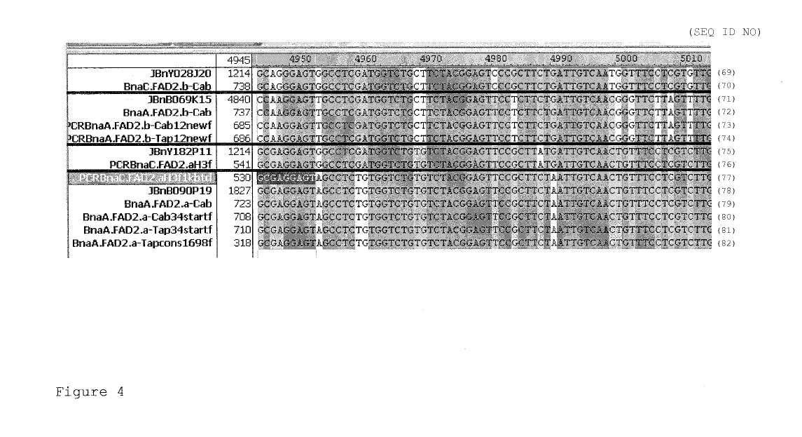

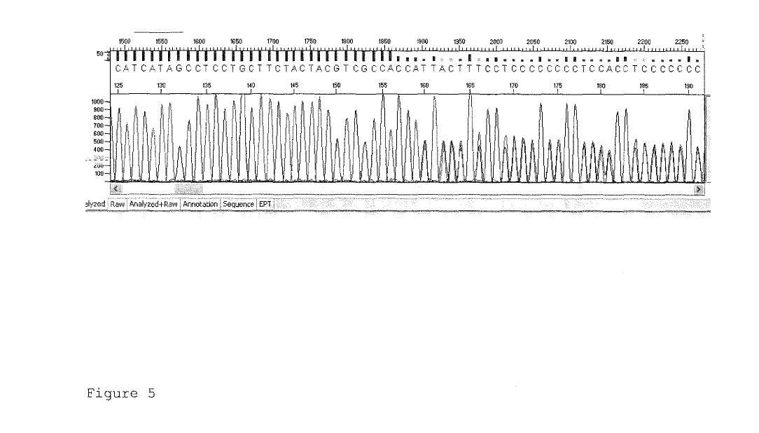

See the alignment of Tapidor and Cabriolet sequences against the different homologues. Note amplification of Cabriolet BnaA.FAD2.a with BnaC.FAD2.a primers under touchdown condition (trace highlighted) in FIG. 4.

Sequencing should be performed on amplicons to determine they are clean. FIG. 5 shows a mixed amplicon.

### Example 2

### EMS Mutation of Brassica Napus Seeds and Development of Mutagenised Population

We utilized B. napus variety Cabriolet for genetic mutagenesis by EMS to produce a starting population to screen. We utilized EMS levels of 0.4, 0.6 and 0.8%. We detected mutations via sequencing BnaC.FAD2.b allele specific PCR product with the forward specific primer (see Example 1 for details, PCR cycles etc.). We aligned sequences and compared mutagenized sequences with wild type sequences using the Mutation Surveyor software. We screened approximately 3000 lines, prioritising the higher EMS treatment levels. The mutagenesis was carried out as follows:

Chemical mutagenesis of ˜25,000 Brassica napus seeds variety Cabriolet using EMS (ethyl methanesulphonate) to produce a mutatagenised population, JBnCAB_E (the JIC consortium Brassica napus Cabriolet EMS population). Before carrying out the method stated here all safety material should be considered. EMS is a mutagen with limited evidence of carcinogenic effects. Equipment required/utilized, includes: Tube rotator (e.g. blood tube rotator—Stuart Scientific) with modified containment box attachment.

Spill tray; 15 ml tubes (Corning BV Cat. No. 430790); Centrifuge tubes (Corning BV Cat. No. 430776); Tea strainer; Dry waste container; Small squares of blue towel; Large liquid waste container; Parafilm (R and L Slaughter Ltd. Cat. No. 291-1214).

Approximately 33000 B. napus seeds var. Cabriolet (breeders seed) were treated. Positive/negative controls: 2.5 g each (˜500 seed) in 15 ml falcon tube. Treatments×5: 30 g each (˜6400 seed) in tubes.

### Reagents Utilized Included:

0.02% Tween 20, Tween 20 (Sigma Aldridge Cat No. TS700-500 ML). Tween 20 is a non-ionic detergent used as a base for the wetting out of seeds for the EMS treatment and for the subsequent washes. It is a very viscous liquid and, therefore, it is probably more accurate to make a higher percentage dilution first and dilute from here. For this protocol a 20% stock solution was produced. Therefore a 1:1000 dilution is required for the 0.02% solution. 1 litre 0.02% Tween 20 is required for the EMS treatment. That is: 1 ml 20% Tween 20 solution made up to 1 litre. 6 litres 0.02% Tween 20 is required for the washes.

10M NaOH, prepared from Sodium hydroxide pellets GPR 1 Kg (Sigma Aldridge Cat. No. 06203). This is used for the neutralisation of the EMS. EMS is inactivated at 1M NaOH. Therefore, a final concentration of 2M can ensure inactivation. Approximately 3000 ml of 10M NaOH is required to neutralise all of the EMS in the initial treatment and subsequent seed washing solutions. MW=40 g; 1M requires 40 g made up to 1 litre. Therefore 10M requires 400 g made up to 1 litre. This production of the solution is highly exothermic and therefore releases a great deal of heat. The NaOH should be added to gently stirring ice cold water and kept on ice until fully dissolved and cool.

EMS (Ethyl methanesulfonate), (Sigma Aldridge Cat. No. M0880). EMS is a well studied chemical mutagen that generates single base pair changes. It is highly toxic and can cause carcinogenic effects. EMS is sold by weight and has a density of 1.16 g/ml. Therefore, 1 g EMS=0.856 ml. Before a full scale mutagenesis experiment, the level and time of exposure should be decided. This can be based on examples in the literature, although the strength of effect of EMS can vary between batches. Therefore a small scale trial is advised. EMS has a short half life (<100 h in aqueous solution @20° C.), therefore the procedure should be carried out with fresh dilutions only. For this work, 5 levels of EMS were determined based on those used for the production of current B. napus mutagenised populations. A positive and a negative control were also included in the work, as follows:

### Negative Control (No EMS):

1×15 ml tube, 2.5 g seed at 0% EMS, 0.02% Tween 20 solution only

### Positive Control (2% EMS):

1×15 ml tube, 2.5 g seed at 2% EMS, 1 ml EMS up to 10 ml with 0.02% Tween 20 solution.

### Treatment 1 (0.2% EMS):

30 g seed, 150 ml solution, 0.3 ml EMS up to 150 ml with 0.02% Tween 20 solution

### Treatment 2 (0.4% EMS):

30 g seed, 150 ml solution, 0.6 ml EMS up to 150 ml with 0.02% Tween 20 solution

### Treatment 3 (0.6% EMS):

30 g seed, 150 ml solution, 0.9 ml EMS up to 150 ml with 0.02% Tween 20 solution

### Treatment 4 (0.8% EMS):

30 g seed, 150 ml solution, 1.2 EMS up to 150 ml with 0.02% Tween 20 solution

### Treatment 5 (1% EMS):

30 g seed, 150 ml solution, 1.5 ml EMS up to 150 ml with 0.02% Tween 20 solution

The EMS treatment was carried out as follows:

### Overnight Treatment:

Set up a work area in a fume hood. Notify everyone in the lab you are about to use EMS and label area clearly. Label tubes clearly. Weigh out 2.5 g seed into the positive and negative control tubes and 30 g into the 5 treatment tubes. Create the treatment solutions by adding the required EMS to the 0.02% Tween 20 solution. Add 12.5 ml of solution to the positive and negative control tubes and 150 ml of solution to the treatment tubes. Catch any drips with the blue paper squares which are disposed of into the dry waste container. Replace the caps, ensure they are tightly closed and seal round the lids with parafilm. Fit into the rotating box and set to turn slowly overnight. Add waste EMS solutions to a 2 litre wide necked disposal bottle containing 400 ml 10M NaOH. Decontaminate the EMS/Tween 20 bottles by adding ˜200 ml 2M NaOH. Seal lid tightly and shake well to ensure that all of the surface has been treated. Leave overnight. Close fume hood and leave overnight.

### EMS Removal and Washes:

Take the two litre disposal bottle from the previous day and slowly decant the EMS treatment solutions into the NaOH. Do this slowly to avoid losing seed or use a tea strainer to prevent seed loss. Rinse the seeds with 0.02% Tween 20 and decant the liquid into the waste bottle. Perform 10 washes of 150 ml 0.02% Tween 20 for 20 minutes/wash, turning slowly. Decant waste into 10MNaOH to give a final volume of 2M NaOH. After the final wash seeds transferred to KWS for sowing. Wallpaper paste was added to the seeds to aid in dispersal the mixture sown by piping onto tray of peat and sand mix. Trays kept at 4° C. for 2 days to stratify before transferring to glasshouse. Seedlings transplanted when large enough to handle.

### Development of the Mutagenised Population

### M₁ Generation

At the four-leaf stage transplanted M₁ seedlings were vernalised at 4° C. for six weeks before transferal to the glasshouse under 16 h day length at 12-18° C., 65-75% relative humidity. Plants were selfed to produce M₂ seed.

### M₂ Generation

Two tramlines of M₂ seed/line of ˜7000 lines were drilled in the field (September 08). One plant/line was selected for tissue sampling and bagged for self seed production. Approximately four leaf disks/selected line were taken and Qiagen DNeasy 96 well DNA preparation (Cat. No. 69181) were performed to produce DNA stocks for mutation screening.

Identification of Mutants with Altered FAD2 Function

Following the genetic analysis described above which determined only one homologue, BnaC.FAD2.b, was functional in Cabriolet, PCR specific for this homologue, as described in Example 1 was performed on M₂ DNA from 3000 lines and sequenced using the standard protocol described previously. Lines containing both heterozygous and homozygous mutations in this homologue were detected by alignment of the sequence against a wild type trace using SoftGenetics Mutation Surveyor DNA Variant Analysis Software.

### Selection, Growth and Phenotyping of M₃ Lines

Twelve M₃ seed from each line containing a mutation was sown and mutation status determined as for the M₂ generation. Where available homozygous, heterozygous and wild type outsegregant lines were trialled in a fully replicated glasshouse trial in the UK under standard B. napus growth conditions to compare oil profiles from the various mutation states. Further replicates of lines were grown for analysis in France. Selfed seed was collected from all lines and analysed for oil profile using NIR. Analysis of fatty acid methyl esters (FAMEs) by gas chromatography (GC) as detailed in Smooker and Wells et al. 2010 can be performed as an alternative.

### Example 3

### Process for Producing Homozygous Plants with No FAD2 Activity

Once the methods disclosed herein are practiced on a given germplasm, a large number of seeds are produced with mutated genotypes. The seeds are germinated and grown, the genome is sequenced and the phenotype confirmed, as shown in FIG. 6. Those new varieties that grow to seed are selfed to produce homozygous plants for field testing and commercial cultivation and oleic acid production.

The crossing and backcrossing of mutant lines with a given conventional germplasm will result in populations segregating for the non-functional alleles of FAD2 present within the mutant lines. PCR and sequencing of the products produced from these lines using the homologue specific primers described within this document will allow the selection of progeny carrying the non-functional alleles and thus these alleles can be tracked through a breeding program. Lines can be phenotyped to confirm the effect of these alleles on the plant phenotype. Lines possessing the non-functional alleles and superior agronomic phenotypes are bulked for field testing, commercial cultivation and oleic acid production.

### Example 4

### Production of Various Germplasms with Altered Fad2 Activity

Following the detailed procedures of Examples 1 and 2, and the more general teachings provided herein above, those skilled in the art will appreciate that, starting with any B. napus germplasm, (and, indeed, other Brassicas) one is able to produce a new germplasm in which the same or different mutations to those disclosed herein for B. napus variety Cabriolet are introduced. This will produce a new B. napus variety in which BnaC.FAD2.b is altered, resulting in little or no activity of the enzyme otherwise encoded by that homologue of the FAD2 gene. By similarly introducing known mutations, truncations, deletions or the like into any remaining FAD2 homologues, such as those already known in the art for such varieties, those skilled in the art are in a position, to rationally produce new varieties of B. napus, in which any or all of the FAD2 activity from any or all of the homologues of the genese encoding that activity has been reduced or removed entirely. Methods of directed gene deletion, silencing and the like, known in the art, may be utilized using the information provided herein, and that already known in the art, to quickly produce desired genotypes and/or, using for example DNA directed siRNA methods, desired phenotypes without even necessarily modifying the genotype. The sequences provided herein may further be used by those skilled in the art to design probes for marker assisted breeding, TILLING and other methods known in the art or which hereafter come into being, to identify and produce varieties of B. napus, and indeed, varieties of other species which have desired oleic acid profiles as compared to other fatty acid compositions.

Based on the disclosure provided herein, and the known art, those skilled in the art are enabled to follow a similar approach in other Brassica to identify the full complement of FAD2 genes in their genomes and to produce, for example, (Brassica juncea, rapa, oleracea, campestris, carinata) in which their full complement of FAD2 genes have been compromised, silenced, mutated, as disclosed herein for the FAD2 genes of Brassica napus.

1. A mutated nucleic acid sequence of a Brassica FAD2-encoding nucleic acid sequence, wherein said nucleic acid sequence has one or more of the mutations shown in FIG. 6 in relation to the BnaC.FAD2.b gene, or a homologous nucleic acid sequence derived from said mutated nucleic acid sequence by substitution, insertion or deletion of at least one nucleotide, and presenting at least 90% homology with said mutated nucleic acid sequence, provided that said homologous nucleic acid sequence does not encode an active FAD2 enzyme, or a hybridizing nucleic acid sequence which hybridizes under stringent conditions to said mutated nucleic acid sequence, provided that the complementary sequence of said hybridizing nucleic acid sequence does not encode an active FAD2 enzyme, or
a complementary sequence of one of said mutated, homologous or hybridizing nucleic acid sequences.

2. The nucleic acid sequence of claim 1 wherein said mutation is one of the following mutations:
224C to T (75 Pro to Leu), 241C to T (81 Leu to Phe), 257G to A (86 Trp to Stop), 258G to A (86 Trp to Stop), 270G to A (90 Trp to Stop), 284G to A (95 Cys to Tyr), 296G to A (99 Gly to Asp), 310G to A (104 Ala to Thr), 316G to A (106 Glu to Lys), 322G to A (108 Gly to Ser), 328C to T (110 His to Tyr), 350G to A (117 Trp to Stop), 355G to A (119 Asp to Asn), 367G to A (123 Gly to Ser), 388C to T (130 Leu to Phe), 421C to T (141 His to Tyr), 425G to A (143 Arg to Gln), 428G to A (143 Arg to His), 437C to T (146 Ser to Phe), 458G to A (153 Arg to Lys), 543G to A (181 Met to Ile), 566G to A (189 Gly to Asp), 570G to A (190 Trp to Stop), 598G to A (200 Gly to Arg), 616G to A (206 Gly to Tyr), 617G to A (206 Gly to Asp), 623C to T (208 Ala to Val), 637C to T (213 Pro to Ser), 642C to A (214 Asn to Lys), 643G to A (215 Ala to Thr), 704C to T (235 Ala to Val), 710G to A (237 Cys to Tyr), 716G to A (239 Gly to Asp), 743G to A (248 Gly to Glu), 776C to T (259 Pro to Leu), 778C to T (260 Leu to Phe), 890C to T (297 Thr to Ile).

3. The nucleic acid sequence of claim 1 or 2 wherein, in relation to the BnaA.FAD2.b gene, the mutation is not one of the mutations shown in FIG. 15.

4. A mutated nucleic acid sequence of a Brassica FAD2-encoding nucleic acid sequence, wherein said nucleic acid sequence encodes a FAD2 enzyme in which amino acid position 241 and/or 246 are changed in relation to the FAD2 enzyme encoded by BnaC.FAD2.b gene, or a homologous nucleic acid sequence derived from said mutated nucleic acid sequence by substitution, insertion or deletion of at least one nucleotide, and presenting at least 90% homology with said mutated nucleic acid sequence, provided that said homologous nucleic acid sequence does not encode an active FAD2 enzyme, or a hybridizing nucleic acid sequence which hybridizes under stringent conditions to said mutated nucleic acid sequence, provided that the complementary sequence of said hybridizing nucleic acid sequence does not encode an active FAD2 enzyme, or
a complementary sequence of one of said mutated, homologous or hybridizing nucleic acid sequences.

5. A mutated nucleic acid sequence of a Brassica FAD2-encoding nucleic acid sequence, wherein said nucleic acid sequence has a 1 bp deletion at coding base position 215 in relation to the BnaA.FAD2.b gene, or
a homologous nucleic acid sequence derived from said mutated nucleic acid sequence by substitution, insertion or deletion of at least one nucleotide, and presenting at least 90% homology with said mutated nucleic acid sequence, provided that said homologous nucleic acid sequence does not encode an active FAD2 enzyme, or a hybridizing nucleic acid sequence which hybridizes under stringent conditions to said mutated nucleic acid sequence, provided that the complementary sequence of said hybridizing nucleic acid sequence does not encode an active FAD2 enzyme, or
a complementary sequence of one of said mutated, homologous or hybridizing nucleic acid sequences.

6. A mutated nucleic acid sequence of a Brassica FAD2-encoding nucleic acid sequence, wherein said nucleic acid sequence has one or more of the mutations shown in FIG. 15 in relation to the BnaC.FAD2.b gene, or a homologous nucleic acid sequence derived from said mutated nucleic acid sequence by substitution, insertion or deletion of at least one nucleotide, and presenting at least 90% homology with said mutated nucleic acid sequence, provided that said homologous nucleic acid sequence encodes an active FAD2 enzyme, or a hybridizing nucleic acid sequence which hybridizes under stringent conditions to said mutated nucleic acid sequence, provided that the complementary sequence of said hybridizing nucleic acid sequence encodes an active FAD2 enzyme, or
a complementary sequence of one of said mutated, homologous or hybridizing nucleic acid sequences.

7. A protein comprising an amino acid sequence encoded by the nucleic acid of any preceding claim.

8. A plant or part thereof in which more than two FAD2-encoding nucleic acid sequences or the corresponding FAD2 enzymes are inactivated such that the enzymatic activity is reduced compared to the non-inactivated version.

9. The plant or part thereof according to claim 8 in which more than three FAD2-encoding nucleic acid sequences or the corresponding FAD2 enzymes are inactivated.

10. The plant or part thereof in which all of the FAD2 enzymes or the nucleic acid sequences encoding therefore are inactivated.

11. The plant or part thereof according to any one of claims 8 to 10 in which the nucleic acid sequence is one or more of those defined in claims 1 to 5.

12. The plant or part thereof according to any one of claims 8 to 11 which is Brassica.

13. The plant of part thereof according to claim 12 which is selected from the group consisting of Brassica juncea, napus, rapa, oleracea, campestris, carinata.

14. The plant or part thereof according to claim 13 which is Brassica napus.

15. The plant or part thereof according to claim 14 which is derivable from the variety Cabriolet or Tapior.

16. The plant or part thereof of any one of claims 13 to 15 in which each of the four FAD2-encoding nucleic acid sequences or the corresponding FAD2 enzymes are inactivated.

17. The plant or part thereof of any one of claims 13 to 16 in which nucleic acid sequences are selected from the group consisting of:
BnaA.FAD2.a
BnaC.FAD2.a
BnaA.FAD2.b or
BnaC.FAD2.b.

18. The plant or a part thereof according to any one of claims 8 to 17 in which the part is a cell or seed.

19. A Brassica seed the fatty acid composition of which is greater, by at least 5%, than 74.5% oleic acid and which also has an 18:2 and/or 18:3 fatty acid fraction which is less by at least 5% than 16.1%.

20. A Brassica seed the fatty acid composition of which is less, by at least 5%, than 74.5% oleic acid and which also has an 18:2 and/or 18:3 fatty acid fraction which is greater by at least 5% than 16.1%.

21. The Brassica seed according to claim 19 or claim 20 wherein said Brassica is selected from the group consisting of Brassica juncea, napus, rapa, oleracea, campestris, carinata.

22. The Brassica seed according to claim 21 which is a Brassica napus seed.

23. Fatty acid produced from the seed or the plant according to any one of claims 8-22.

24. A lubricant comprising the fatty acid of claim 23.

25. A method of selecting plants having a desired fatty acid content comprising the following steps:
crossing a plant or part thereof of the present invention with a parent plant;
selecting from the progeny plants or parts thereof those comprising at least one sequence of any one of claims 1 to 6.

26. A method of providing a plant having increased monounsaturated fatty acid content, particularly oleic acid content, comprising the steps of:
inactivating at least one FAD2-encoding nucleic acid sequence in a plant or part thereof,
selecting a plant or part thereof or regenerated plant comprising said inactivation.

27. A method of producing a Brassica plant or a part thereof in which FAD2 is inactivated, said method selected from the group consisting of (a) silencing each of the homologues of the gene encoding FAD2 (b) producing mutants in the genome to reduce or destroy the activity of any encoded enzyme (c) selecting a variety of Brassica in which only one active FAD2 gene exists, mutating that gene and producing a new variety in which all genes encoding oleate-12 desaturase are modified as compared with wild-type of that variety such that any oleate desaturase protein encoded is compromised in activity or is devoid of activity and (d) marker assisted selection of crosses between particular varieties to produce a variety which encodes little or no active FAD2 enzymatic activity.

28. A method of providing a plant having increased PUFA content, particularly linoleic acid and/or linolenic fatty acid content, comprising the steps of:
activating at least one FAD2-encoding nucleic acid sequence of the present invention in a plant or part thereof,
selecting a plant or part thereof or regenerated plant comprising said activation.

29. A method of producing a plant or a part thereof, said method selected from the group consisting of (a) upregulating expression of more or more of the homologues of the gene encoding FAD2 (b) producing mutants in the genome to increase the activity of any encoded FAD2 (c) selecting a variety of Brassica in which a FAD2 gene exists, mutating that gene and producing a new variety in which a gene and preferably all genes encoding oleate-12 desaturase are modified as compared with wild-type of that variety such that any oleate desaturase protein encoded is improved in activity and (d) marker assisted selection of crosses between particular varieties to produce a variety which encodes active FAD2 enzymatic activity.

30. The method of any one of claims 25 to 29 wherein said plant or part thereof is Brassica.

31. The method according to claim 30 wherein said Brassica is selected from the group consisting of Brassica juncea, napes, rapa, oleracea, campestris, carinata.

32. The method according to claim 31 wherein said Brassica is Brassica napes.

33. A nucleic acid sequence which is identical in sequence to a portion of a modified BnaC.FAD2.b gene.

34. A nucleic acid sequence comprising at least 5 contiguous wild-type bases on either side of the mutation according to any one of claims 1 to 6.

35. The nucleic acid sequence selected from the group consisting of
SEQ ID. 52:
GTCTCCTCCCTCCAAAAAGT
SEQ ID. 53:
GTGTCTCCTCCCTCCAAA
SEQ ID. 54:
CTACAGAAACAAACATGGGC
SEQ ID. 55:
GTCTCTCCTCCCTCCAGC
SEQ ID. 56:
CTCTCCTCCCTCCAGCTCCC
SEQ ID. 57:
CTCTTCGACATCCTCCTCTC
SEQ ID. 58:
CCTCGTCCCTTACTTCTCCTG
SEQ ID. 59:
CCTCATAACTTATTGTTGTACCAG
SEQ ID. 60:
CAAGACGACCAGAGACAGC
SEQ ID. 61:
GAACTCGACAAATTTGCCTG
SEQ ID. 62:
GGGTGCAGGTGGAAGAATG probe f,
SEQ ID. 63:
TTGTTGTACCAGTACACACC probe r,
SEQ ID. 64:
conserved f GAGGGAGGCGAAGGAGTGTATC,
SEQ ID. 65:
conserved r CAGGAGAAGTAAGGGACGAGG,
SEQ ID. 66:
degenerate f ATTCCTTCCTNCTNCTNGTNCC,
SEQ ID. 67:
degenerate r GCTAAGTACAANGGNCANCC.

36. Use of a nucleic acid sequence according to claim 35 in combination with a primer or probe which is identical in sequence to a portion of a modified BnaA.FAD2.b, BnaC.FAD2a or BnaA.FAD2.a gene.
